(19) United States
(12) Patent Application Publication
(10) Pub. No.: US 2009/0253673 A1
(43) Pub. Date: Oct. 8, 2009

(54) Substituted Pyrazoles as Ghrelin Receptor Antagonists

(76) Inventors: Min Ge, Edison, NJ (US); Eric Cline, Princeton, NJ (US); Lihu Yang, Edison, NJ (US); Sander G. Mills, Scotch Plains, NJ (US)

Correspondence Address: MERCK AND CO., INC, P O BOX 2000, RAHWAY, NJ, 07065-0907, US

(21) Appl. No.: 12/227,545

(22) PCT Filed: Jul. 6, 2007
(86) PCT No.: PCT/US2007/015597

§ 371 Date: Nov. 20, 2008

Related U.S. Application Data
(60) Provisional application No. 60/830,260, filed on Jul. 12, 2006.

(51) Int. Cl. A61K 31/4155; C07D 403/06; C07D 401/06; C07D 231/12; C07D 231/38; C07D 413/12; C07D 417/12; A61K 31/415; A61K 31/454; A61K 31/496; A61K 31/4178; A61K 31/422; A61K 31/4245; A61K 31/4439; A61K 31/497; A61K 31/506; A61K 31/427; A61P 3/04; A61P 3/10

(52) U.S. Cl. 514/210.2; 548/364.1; 546/211; 544/371; 548/374.1; 548/364.7; 548/371.7; 548/236; 546/275.4; 544/333; 548/200; 514/406; 514/326; 514/254.05; 514/407; 514/397; 514/374; 514/364; 514/341; 514/255.05; 514/256; 514/365

(57) ABSTRACT

Certain novel N-acylated spiropiperidine derivatives are ligands of the human ghrelin receptor(s) and, in particular, are antagonists/inverse agonists of the human ghrelin receptor. They are therefore useful for the treatment, control, or prevention of diseases and disorders responsive to the modulation of the ghrelin receptor, such as obesity, diabetes, and metabolic syndrome.

### BACKGROUND OF THE INVENTION

Obesity is a major health concern in Western societies. It is estimated that about 97 million adults in the United States are overweight or obese. Epidemiological studies have shown that increasing degrees of overweight and obesity are important predictors of decreased life expectancy. Obesity causes or exacerbates many health problems, both independently and in association with other diseases. The medical problems associated with obesity, which can be serious and life-threatening, include hypertension; type 2 diabetes mellitus; elevated plasma insulin concentrations; insulin resistance; dyslipidemias; hyperlipidemia; endometrial, breast, prostate and colon cancer; osteoarthritis; respiratory complications, such as obstructive sleep apnea; cholelithiasis; gallstones; arteriosclerosis; heart disease; abnormal heart rhythms; and heart arrythmias (Kopelman, P. G., Nature 404, 635-643 (2000)). Obesity is further associated with premature death and with a significant increase in mortality and morbidity from stroke, myocardial infarction, congestive heart failure, coronary heart disease, and sudden death.

Ghrelin was identified as an endogenous ligand, synthesized primarily in the stomach, for the growth hormone secretagogue receptor (GHS-R) in 1999. Ghrelin is a small 28 amino acid peptide with an acyl side chain required for biological activity (Kojima et al., Nature, 402, 656-660, 1999). Ghrelin has been shown to stimulate growth hormone (GH) release and also to increase food intake when administered both centrally and peripherally (Wren et al., Endocrinology, 141, 4325-4328, 2000). Ghrelin is thought to signal pre meal hunger.

Endogenous levels of ghrelin rise on fasting and fall on re-feeding in man (Cummings et al., Diabetes, 50, 1714-1719, 2001). Ghrelin also appears to play a role in long term energy balance and appetite regulation. Chronic administration of ghrelin in rodents leads to hyperphagia and weight gain that are independent of growth hormone secretion (Tschop et al., Nature, 407, 908-913, 2000). Circulating ghrelin levels decrease in response to chronic overfeeding and increase in response to chronic negative energy balance associated with anorexia or exercise. Obese people generally have low plasma ghrelin levels (Tschop et al., Diabetes, 50, 707-709, 2001). Intravenous ghrelin is effective in stimulating food intake in humans. A recent study showed a 28% food intake increase from a buffet meal with a ghrelin infusion compared with saline control (Wren et al., J Clin Endocrinology and Metabolism, 86, 5992, 2001). Based on these studies, an antagonist at the ghrelin growth hormone secretagogue (GHS-R) receptor may be an obesity treatment. A selective antagonist at the GHS receptor would reduce appetite, reduce food intake, induce weight loss and treat obesity without affecting or significantly reducing the circulating growth hormone levels.

Weight loss drugs that are currently used in monotherapy for the treatment of obesity have limited efficacy and significant side effects. There is a need for a weight loss treatment with enhanced efficacy and fewer undesirable side effects. The instant invention addresses this problem by providing antagonists/inverse agonists of the ghrelin receptor, useful in the treatment and prevention of obesity and obesity-related disorders, including diabetes.

Compositions of ghrelin antagonists and inverse agonists, and/or growth hormone secretagogue receptor antagonists, and methods for the treatment of obesity are disclosed in U.S. Patent Publication Nos. US 2005/0014794, US 2005/0070712, US 2005/0171131, US 2005/0171132, and in WO 2005/035498, WO 2005/030734, WO 2005/012331, and WO 2005/012332.

### SUMMARY OF THE INVENTION

The present invention relates to novel substituted pyrazoles of structural formula I:

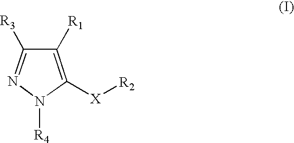

The compounds of structural formula I are effective as ghrelin receptor antagonists/inverse agonists and are particularly effective as antagonists and/or inverse agonists of the ghrelin receptor. They are therefore useful for the treatment and/or prevention of disorders responsive to the modulation of the ghrelin receptor, such as obesity, diabetes, metabolic syndrome and obesity-related disorders.

The present invention also relates to pharmaceutical compositions comprising the compounds of the present invention and a pharmaceutically acceptable carrier.

The present invention also relates to methods for the treatment or prevention of disorders, diseases, or conditions responsive to the modulation of the ghrelin receptor in a mammal in need thereof by administering the compounds and pharmaceutical compositions of the present invention.

The present invention further relates to the use of the compounds of the present invention in the preparation of a medicament useful for the treatment or prevention of disorders, diseases, or conditions responsive to the modulation of the ghrelin receptor in a mammal in need thereof by administering the compounds and pharmaceutical compositions of the present invention.

### DETAILED DESCRIPTION OF THE INVENTION

The present invention relates to substituted pyrazole derivatives useful as ghrelin receptor modulators, in particular, as ghrelin receptor antagonists/inverse agonists. Compounds of the present invention are described by structural formula I:

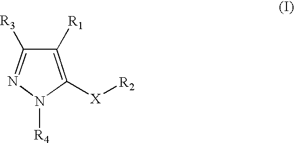

or a pharmaceutically acceptable salt thereof; wherein
X is selected from the group consisting of:

(1) bond,

(2) —(CH₂)ₘ—,

(3) —(CH₂)ₘC₂₋₆heterocycloalkyl-,

(4) —(CH₂)ₙC₂₋₆heterocycloalkyl-(CH₂)ₙ—NR⁶—,

(5) —NR⁶—(CH₂)ₙC₃₋₆cycloalkyl-(CH₂)ₙ—NR⁶—,

(6) —(CH₂)ₘNR⁶—,

(7) —NR⁶—(CH₂)ₘ—,

(8) —(CH₂)ₙ—NR⁶—(CH₂)ₘ—NR⁶—,

(9) —NR⁶—C₂₋₆alkenyl-,

(10) —NR⁶—C₂₋₆alkynyl-,

(11) —NR⁶-phenyl-,

(12) —NR⁶-phenyl-NR⁶—,

(13) —NR⁶—(CH₂)ₙ—C₂₋₆heterocycloalkyl-,

(14) —NR⁶—(CH₂)ₙ-heteroaryl-, and

(15) —NR⁶-heteroaryl-NR⁶—,

wherein alkenyl, alkynyl, cycloalkyl, heterocycloalkyl, phenyl, heteroaryl, and (CH₂) are unsubstituted or substituted with 1-4 substituents selected from oxo, halogen and C₁₋₄alkyl;
R¹ is selected from the group consisting of

(1) hydrogen,

(2) —CF₃,

(3) halogen,

(4) —C₁₋₈alkyl,

(5) —C₂₋₈alkenyl,

(6) —C₂₋₈alkynyl,

(7) —(CH₂)ₙOH,

(8) —(CH₂)ₙphenyl,

(9) —(CH₂)ₙheteroaryl,

(10) —(CH₂)ₙC₃₋₇cycloalkyl,

(11) —(CH₂)ₙC₂₋₉heterocycloalkyl,

(12) —(CH₂)ₙN(R⁶)CH₂phenyl,

(13) —(CH₂)ₙN(R⁶)C(O)phenyl,

(14) —(CH₂)ₙN(R⁶)C(O)heteroaryl,

(15) —CN,

(16) —C(O)R⁵,

(17) —C(O)C₂₋₈alkenyl,

(18) —C(O)C₂₋₈alkynyl,

(19) —C(O)C₃₋₇cycloalkyl,

(20) —C(O)C₂₋₉heterocycloalkyl,

(21) —CO₂R⁵,

(22) —C(O)N(R⁶)₂, and

(23) —(CH₂)₃₋₇R²,

wherein alkyl, alkenyl, alkynyl, phenyl, heteroaryl, heterocycloalkyl, and cycloalkyl are unsubstituted or substituted with one to three groups independently selected from CF₃, C₁₋₄ alkoxy, C₁₋₄ alkyl, halogen and phenyl, wherein the phenyl substituent is unsubstituted or substituted with CF₃, C₁₋₄ alkoxy, C₁₋₄ alkyl and halogen;
R² is selected from the group consisting of

(1) hydrogen,

(2) —C₁₋₈alkyl,

(3) —C₂₋₈alkenyl,

(4) —C₂₋₈alkynyl,

(5) —(CH₂)ₙC₃₋₇cycloalkyl,

(6) —(CH₂)ₙC₂₋₉heterocycloalkyl,

(7) —(CH₂)ₙphenyl,

(8) —(CH₂)ₙnaphthyl,

(9) —(CH₂)ₙheteroaryl,

(10) —OR⁶,

(11) —C(O)R⁶,

(12) ═CH—NR⁶)₂,

(13) —(CH₂)ₙN(R⁶)₂,

(14) —(CH₂)ₙN(R⁶)CO₂C₁₋₈alkyl,

(15) —(CH₂)ₙCO₂H,

(16) —C(O)C₁₋₈alkyl,

(17) —C(O)C₃₋₇cycloalkyl,

(18) —C(O)C₂₋₉heterocycloalkyl,

(19) —C(O)(CH₂)ₙaryl,

(20) —C(O)(CH₂)ₙheteroaryl,

(21) —C(O)CF₃,

(22) —C(O)(CH₂)ₙN(R⁶)₂,

(23) —C(O)N(R⁶)C₁₋₈alkyl,

(24) —C(O)N(R⁶)(CH₂)ₙC₃₋₇cycloalkyl,

(25) —C(O)N(R⁶)(CH₂)ₙC₂₋₇heterocycloalkyl,

(26) —C(O)N(R⁶)(CH₂)ₙphenyl,

(27) —C(O)N(R⁶)(CH₂)ₙnaphthyl,

(28) —C(O)N(R⁶)(CH₂)ₙheteroaryl,

(29) —C(S)N(R⁶)(CH₂)ₙphenyl,

(30) —CO₂C₁₋₈alkyl,

(31) —CO₂(CH₂)ₙC₃₋₇cycloalkyl,

(32) —CO₂(CH₂)ₙC₂₋₉heterocycloalkyl

(33) —CO₂(CH₂)ₙphenyl,

(34) —CO₂(CH₂)ₙnaphthyl,

(35) —CO₂(CH₂)ₙheteroaryl,

(36) —SO₂C₁₋₈alkyl,

(37) —SO₂C₃₋₇cycloalkyl,

(38) —SO₂C₂₋₉heterocycloalkyl,

(39) —SO₂phenyl,

(40) —SO₂naphthyl,

(41) —SO₂heteroaryl,

(42) —S(O)N(R⁶)phenyl,

(43) —S—C₁₋₈alkyl,

(44) —S—C₃₋₇cycloalkyl,

(45) —S—C₂₋₉heterocycloalkyl,

(46) —S-phenyl,

(47) —S-naphthyl, and

(48) —S-heteroaryl,

wherein alkyl, alkenyl, alkynyl, cycloalkyl, heterocycloalkyl, aryl, phenyl, naphthyl, heteroaryl, and (CH₂) are unsubstituted or substituted with one to four substituents independently selected from R⁷, and wherein two C₁₋₄ alkyl substituents on the same (CH₂) carbon may cyclize to form a 3- to 6-membered ring, provided that when X is a bond or —(CH₂)ₘ then R² is not hydrogen, —C₁₋₈alkyl, —C₂₋₈alkenyl, —C₂₋₈alkynyl, —(CH₂)ₙC₃₋₇cycloalkyl, —C₂₋₉heterocycloalkyl, -phenyl, -benzyl, -naphthyl, -heteroaryl, —OR⁶, —C(O)R⁶, or —S—C₁₋₈alkyl, further provided that when X is a bond R² is not —NH₂, —CO₂C₁₋₈alkyl, —CO₂C₃₋₇cycloalkyl, —CO₂(CH₂)₀₋₁phenyl, and provided that when X is —(CH₂)ₘNR⁶— then R² is not —C(O)R⁶;
R³ is selected from the group consisting of:

(1) —C₁₋₈alkyl,

(2) —(CH₂)ₙ-phenyl,

(3) —(CH₂)ₙ-naphthyl,

(4) —(CH₂)ₙC₃₋₇cycloalkyl,

(5) —C(O)C₁₋₈alkyl,

(6) —CO₂R⁵,

(7) —C(O)N(R⁶)OC1-8alkyl,

(8) —C(O)C₁₋₄alkenylphenyl,

(9) —C(O)C₁₋₄alkynylphenyl,

(10) —C(O)phenyl,

(11) —C(O)naphthyl,

(12) —C(O)heteroaryl, and

(13) —C(O)C₃₋₇cycloalkyl,

wherein alkyl, alkenyl, alkynyl, phenyl, naphthyl, heteroaryl, and cycloalkyl are unsubstituted or substituted with one to three groups independently selected from R⁸, and each (CH₂)ₙ is unsubstituted or substituted with 1 to 2 groups independently selected from: C₁₋₄alkyl, —OH, halogen, and C₁₋₄ alkenyl;
R⁴ is selected from the group consisting of:

(1) —(CH₂)ₙ-phenyl,

(2) —(CH₂)ₙ-naphthyl,

(3) —(CH₂)ₙ-heteroaryl,

(4) —(CH₂)ₙC₂₋₉heterocycloalkyl,

(5) —(CH₂)ₙC₃₋₇cycloalkyl, and

(6) —S(O)₂phenyl;

wherein phenyl, naphthyl, heteroaryl, heterocycloalkyl, cycloalkyl and (CH₂) are unsubstituted or substituted with one to three groups independently selected from R⁹;
each R⁵ is independently selected from the group consisting of

(1) —C₁₋₈alkyl,

(2) —(CH₂)ₙphenyl, and

(3) —(CH₂)ₙheteroaryl,

wherein each carbon in —C₁₋₈alkyl is unsubstituted or substituted with one to three groups independently selected from C₁₋₄alkyl;
each R⁶ is independently selected from the group consisting of

(1) hydrogen,

(2) —C₁₋₈ alkyl,

(3) —C₂₋₈alkenyl,

(4) —C₂₋₈alkynyl,

(5) (CH₂)ₙphenyl,

(6) —C₂₋₈alkenyl-phenyl, and

(7) —(CH₂)ₙCO₂H,

wherein alkyl, alkenyl, alkynyl and (CH₂)ₙ are unsubstituted or each carbon is substituted with 1 or 2 substituents independently selected from —OC₁₋₄alkyl, and —C₁₋₄alkyl; and phenyl is unsubstituted or substituted with 1-3 groups selected from —OC₁₋₄alkyl, and —C₁₋₄alkyl;
each R⁷ is independently selected from the group consisting of:

(1) halogen,

(2) oxo,

(3) ═NH,

(4) —CN,

(5) —CF₃,

(6) —OCF₃,

(7) —C₁₋₆ alkyl,

(8) —C₂₋₆ alkenyl,

(9) —C₂₋₆ alkynyl,

(10) —(CH₂)ₙC₃₋₆cycloalkyl,

(11) —(CH₂)ₙC₂₋₉heterocycloalkyl,

(12) —(CH₂)ₙOR⁶,

(13) —(CH₂)ₙCO₂R⁶,

(14) —(CH₂)ₙCO₂(CH₂)ₙphenyl;

(15) —(CH₂)ₙphenyl;

(16) —(CH₂)ₙ—O-phenyl;

(17) —(CH₂)ₙnaphthyl,

(18) —(CH₂)ₙ-heteroaryl,

(19) —N(R⁶)₂,

(20) —NR⁶C(O)R⁶,

(21) —NR⁶C(O)₂R⁶,

(22) —C(O)phenyl,

(23) —C(O)heteroaryl,

(24) —SR⁵,

(25) —SO₂C₁₋₆alkyl, and

(26) —SO₂N(R⁶)₂,

wherein alkyl, alkenyl, alkynyl, phenyl, heteroaryl, heterocycloalkyl, naphthyl, cycloalkyl, and (CH₂)ₙ are unsubstituted or substituted with one to three groups independently selected from oxo, halogen, C₁₋₄ alkyl and OR⁵;
each R⁸ is independently selected from the group consisting of:

(1) —C₁₋₆alkyl,

(2) —C₁₋₆alkenyl,

(3) —C₁₋₆alkynyl,

(4) —C₁₋₆alkoxy,

(5) —C₃₋₆cycloalkyl,

(6) —(CH₂)ₙ-phenyl, unsubstituted or substituted with halogen,

(7) —O—(CH₂)ₙ-phenyl,

(8) —CN,

(9) —OH,

(10) halogen,

(11) —CF₃,

(12) —NH₂,

(13) —N(C₁₋₆alkyl)₂,

(14) —NO₂, and

(15) —SC₁₋₆alkyl;

each R⁹ is independently selected from the group consisting of:

(1) halogen,

(2) —C₁₋₆alkyl,

(3) —C₂₋₆alkenyl,

(4) —C₂₋₆alkynyl,

(5) phenyl,

(6) —CH₂phenyl,

(7) —(CH₂)ₙOR⁶,

(8) —CN,

(9) —OCF₃,

(10) —CF₃,

(11) —NO₂,

(12) —NR⁵COR⁵,

(13) —CO₂R⁵, and

(14) —CO₂H;

n is 0, 1, 2, 3, 4, 5, 6, 7 or 8; and
m is 1, 2, 3, 4, 5, 6, 7 or 8.

In one embodiment of the compounds of structural formula I, X is selected from the group consisting of: bond, —(CH₂)ₘ—, —(CH₂)ₘC₂₋₆heterocycloalkyl-, —(CH₂)ₙC₂₋₆heterocycloalkyl-(CH₂)ₙ—NR⁶—, —NR⁶—(CH₂)ₙC₃₋₆cycloalkyl-NR⁶—, —(CH₂)ₘNR⁶—, —NR⁶—(CH₂)ₘ—, —NR⁶—(CH₂)ₘ—NR⁶—, —NR⁶—C₂₋₆alkenyl-, —NR⁶-phenyl-, —NR⁶-phenyl-NR⁶—, —NR⁶—(CH₂)ₙ—C₂₋₆heterocycloalkyl-, and —NR⁶—(CH₂)ₙ-heteroaryl-, wherein alkenyl, alkynyl, cycloalkyl, heterocycloalkyl, phenyl, heteroaryl, and (CH₂)ₙ are unsubstituted or substituted with 1-4 substituents selected from oxo, halogen and C₁₋₄alkyl. In a class of this embodiment, X is selected from the group consisting of: —(CH₂)ₘC₂₋₆heterocycloalkyl-, —(CH₂)ₙC₂₋₆heterocycloalkyl-(CH₂)ₙ—NR⁶—, —NR⁶—(CH₂)ₙC₃₋₆cycloalkyl-(CH₂)ₙ—NR⁶—, —(CH₂)ₘNR⁶—, —NR⁶—(CH₂)ₘ—, —NR⁶—(CH₂)ₘ—NR⁶—, —NR⁶—C₂₋₆alkenyl-, —NR⁶-phenyl-NR⁶—, —NR⁶—C₂₋₆heterocycloalkyl-, and —NR⁶—(CH₂)ₙ-heteroaryl-, wherein alkenyl, alkynyl, cycloalkyl, heterocycloalkyl, phenyl, heteroaryl, and (CH₂)ₙ are unsubstituted or substituted with 1-4 substituents selected from oxo, halogen and C₁₋₄alkyl. In a subclass of this class, X is selected from the group consisting of: —CH₂-piperazinyl-, —CH₂-pyrrolidinyl-NH—, —CH₂-azetidinyl-NH—, -azetidinyl-CH₂—NH—, —NH-cyclobutyl-NH—, —NH—(CH₂)₂—, —NH—(CH₂)₃—NH—, —NH-phenyl-NH—, and —(CH₂)NH-pyrrolidinyl-. In another class of this embodiment, X is selected from the group consisting of: —(CH₂)ₙ-heterocycloalkyl-NR⁶—, —NR⁶—C₃₋₆cycloalkyl-NR⁶—, —NR⁶—(CH₂)ₘ—NR⁶—, and —NR⁶—(CH₂)ₙ—, wherein heterocycloalkyl, heteroaryl, and (CH₂)ₙ are unsubstituted or substituted with 1-4 substituents selected from oxo, halogen and C₁₋₄alkyl. In a subclass of this class, X is selected from the group consisting of: —(CH₂)-pyrrolidinyl-NH—, —NH-cyclobutyl-NH—, —NH—(CH₂)₃—NH—, and —NH—(CH₂)₃—.

In another embodiment of the compounds of structural formula I, R¹ is selected from the group consisting of: —CF₃, halogen, —C₁₋₈alkyl, —(CH₂)ₙOH, —(CH₂)ₙphenyl, —(CH₂)ₙheteroaryl, —(CH₂)ₙN(R⁶)CH₂phenyl, —(CH₂)ₙN(R⁶)C(O)phenyl, —(CH₂)ₙN(R⁶)C(O)heteroaryl, —CN, —CO₂R⁵, and —C(O)N(R⁶)₂, wherein alkyl, phenyl, heteroaryl and (CH₂)ₙ are unsubstituted or substituted with one to three groups independently selected from CF₃, C₁₋₄ alkoxy, C₁₋₄ alkyl, halogen, and phenyl unsubstituted or substituted with CF₃, C₁₋₄ alkoxy, C₁₋₄alkyl and halogen. In a class of this embodiment, R¹ is selected from the group consisting of: —CF₃, halogen, —C₁₋₈alkyl, —(CH₂)₃OH, -tetrazole, —(CH₂)ₙN(H)CH₂phenyl, —(CH₂)ₙN(R⁶)C(O)phenyl, —(CH₂)ₙN(R⁶)C(O)heteroaryl, —CN, —CO₂R⁵, and —C(O)N(R⁶)₂, wherein alkyl, phenyl, heteroaryl and (CH₂)ₙ are unsubstituted or substituted with one to three groups independently selected from CF₃, C₁₋₄ alkoxy, C₁₋₄ alkyl, halogen, and phenyl unsubstituted or substituted with CF₃, C₁₋₄ alkoxy, C₁₋₄ alkyl and halogen. In a subclass of this class, R¹ is selected from the group consisting of: halogen, —C₁₋₄alkyl, and —CN. In another subclass of this class, R¹ is halogen or —CN. In a subclass of this subclass, R¹ is —CN.

In another embodiment of the compounds of structural formula I, R¹ is selected from the group consisting of: —(CH₂)ₙheteroaryl and —CN, wherein heteroaryl and (CH₂)ₙ are unsubstituted or substituted with one to three groups independently selected from CF₃, C₁₋₄ alkoxy, C₁₋₄ alkyl, halogen, and phenyl unsubstituted or substituted with CF₃, C₁₋₄ alkoxy, C₁₋₄ alkyl and halogen. In a class of this embodiment, R¹ is tetrazole.

In another embodiment of the present invention, R² is selected from the group consisting of: hydrogen, —C₁₋₈alkyl, —(CH₂)ₙC₂₋₉heterocycloalkyl, —(CH₂)ₙphenyl, —(CH₂)ₙnaphthyl, —(CH₂)ₙheteroaryl, —OR⁶, —(CH₂)ₙN(R⁶)₂, —(CH₂)ₙN(R⁶)CO₂C₁₋₈alkyl, —C(O)C₁₋₈alkyl, —C(O)C₃₋₇cycloalkyl, —C(O)C₂₋₉heterocycloalkyl, —C(O)(CH₂)ₙaryl, —C(O)(CH₂)ₙheteroaryl, —C(O)CF₃, —C(O)(CH₂)ₙN(R⁶)₂, —C(O)N(R⁶)C₁₋₈alkyl, —C(O)N(R⁶)(CH₂)ₙC₃₋₇cycloalkyl, —C(O)N(R⁶)(CH₂)ₙC₂₋₇heterocycloalkyl, —C(O)N(R⁶)(CH₂)ₙphenyl, —C(O)N(R⁶)(CH₂)ₙnaphthyl, —C(O)N(R⁶)(CH₂)ₙ-heteroaryl, —C(S)N(R⁶)(CH₂)ₙphenyl, —CO₂C₁₋₈alkyl, —CO₂(CH₂)ₙphenyl, —SO₂C₁₋₈alkyl, —SO₂phenyl, —S(O)N(R⁶)phenyl, and —S-phenyl, wherein alkyl, cycloalkyl, heterocycloalkyl, aryl, phenyl, naphthyl, heteroaryl, and (CH₂) are unsubstituted or substituted with one to four substituents independently selected from R⁷, and wherein two C₁₋₄ alkyl substituents on the same (CH₂) carbon may cyclize to form a 3- to 6-membered ring, provided that when X is a bond or —(CH₂)ₘ then R² is not hydrogen, —C₁₋₈alkyl, —C₂₋₈alkenyl, —C₂₋₈alkynyl, —(CH₂)ₙC₃₋₇cycloalkyl, —C₂₋₉heterocycloalkyl, -phenyl, -benzyl, -naphthyl, -heteroaryl, —OR⁶, —C(O)R⁶, or —S—C₁₋₈alkyl, further provided that when X is a bond R² is not —NH₂, —CO₂C₁₋₈alkyl, —CO₂C₃₋₇cycloalkyl, —CO₂(CH₂)₀₋₁phenyl, and provided that when X is —(CH₂)ₘNR⁶— then R² is not hydrogen or —C(O)R⁶. In a class of this embodiment, R² is selected from the group consisting of: hydrogen, —C₁₋₈alkyl, —(CH₂)ₙC₂₋₉heterocycloalkyl, —(CH₂)ₙphenyl, —(CH₂)ₙnaphthyl, —(CH₂)ₙheteroaryl, —OR⁶, —(CH₂)ₙN(R⁶)₂, —(CH₂)ₙN(R⁶)CO₂C₁₋₈alkyl, —C(O)C₁₋₈alkyl, —C(O)C₃₋₇cycloalkyl, —C(O)C₂₋₉heterocycloalkyl, —C(O)(CH₂)ₙaryl, —C(O)(CH₂)ₙheteroaryl, —C(O)CF₃, —C(O)N(R⁶)C₁₋₈alkyl, —C(O)N(R⁶)(CH₂)ₙphenyl, —C(O)N(R⁶)(CH₂)ₙnaphthyl, —CO₂C₁₋₈alkyl, —CO₂(CH₂)ₙphenyl, —SO₂C₁₋₈alkyl, —SO₂phenyl, —S(O)N(R⁶)phenyl, and —S-phenyl, wherein alkyl, cycloalkyl, heterocycloalkyl, aryl, phenyl, naphthyl, heteroaryl, and (CH₂) are unsubstituted or substituted with one to four substituents independently selected from R⁷, and wherein two C₁₋₄ alkyl substituents on the same (CH₂) carbon may cyclize to form a 3- to 6-membered ring, provided that when X is a bond or —(CH₂)ₘ then R² is not hydrogen, —C₁₋₈alkyl, —C₂₋₈alkenyl, —C₂₋₉heterocycloalkyl, -phenyl, -benzyl, -naphthyl, -heteroaryl, —OR⁶, —C(O)R⁶, or —S—C₁₋₈alkyl, further provided that when X is a bond R² is not —NH₂, —CO₂C₁₋₈alkyl, —CO₂(CH₂)₀₋₁phenyl, and provided that when X is —(CH₂)ₘNR⁶— then R² is not hydrogen or —C(O)R⁶. In a subclass of this class, R² is selected from the group consisting of: —(CH₂)ₙphenyl, —(CH₂)ₙheteroaryl, —C(O)phenyl, and —C(O)heteroaryl, wherein phenyl and heteroaryl are unsubstituted or substituted with one to three substituents independently selected from R⁷, and wherein each (CH₂) carbon is unsubstituted or substituted with one or two substituents independently selected from halogen, C₁₋₄alkyl, oxo, —(CH₂)ₙOR⁵, —(CH₂)ₙCO₂R⁵, or two C₁₋₄ alkyl substituents on the same (CH₂) carbon can cyclize to form a 3- to 6-membered ring; provided that when X is a bond or —(CH₂)ₘ then R² is not -phenyl, -benzyl, -heteroaryl, or —C(O)R⁶, and provided that when X is —(CH₂)ₘNR⁶— then R² is not hydrogen or —C(O)R⁶. In a subclass of this subclass, R² is selected from the group consisting of: —CH₂phenyl, 6-methoxy-3,4-dihydro-2H-isoquinoline-1-one, —C(O)phenyl substituted with OCH₃, and —C(O)-indole, provided that when X is a bond or —(CH₂)ₘ then R² is not -phenyl, —C(O)phenyl or —C(O)indole, and provided that when X is —(CH₂)ₘNR⁶— then R² is not hydrogen, —C(O)phenyl or C(O)indole. In another class of this embodiment, heterocycloalkyl is selected from the group consisting of: azetidine, aziridine, pyrrolidine, piperazine, morpholine, piperidine, piperidin-2-one, 2-azabicyclo[2.2.1]heptane, 1,4-tetrahydropyran, and octahydro-pyrrolo[1,2-a]pyrazine, 1,3-dioxane, and 1,4-tetrahydropyran. In another class of this embodiment, heteroaryl is selected from the group consisting of: phthalimide, indole, pyridine, pyrimidine, benzimidazole, 3H-benzothiazol-2-ylideneamine, 1,2,3,4 tetrahydro-isoquinoline, 5,6,7,8 tetrahydroimidazo[1,2-a]pyrazine-2-one, 1-oxo-2,3,4 trihydroisoquinoline, 3,4-Dihydro-2H-isoquinolin-1-one, 7,8-Dihydro-6H-[1,6]naphthyridin-5-one, 6,7-dihydro-pyrrolo[3,4-b]pyridin-5-one, 2,3-Dihydro-isoindol-1-one, 2,3-Dihydro-benzo[e][1,3]oxazin-4-one, 2,3-Dihydro-pyrido[3,2-e][1,3]oxazin-4-one, pyrazolo[1,5-a]pyridine, imidazo[1,2-a]pyridine, quinolinone, benzo-[1,2,3]thiadiazole, benzo[1,2,5]thiadiazole, thiene, indoline, indole, benzothiazole, benzothiophene, 1,4 benzodioxan, benzimidazole, benzotriazole, benzoxazole, benzofuran, 1,4-benzothiadiazole, benz-oxadiazole, 1,2,5 oxadiazole, 1,2,4 oxadiazole, thiazole, triazole, 1,2,4 triazole, 1,2,3 triazole, oxazole, isoxazole, imidazole, pyrazole, pyrazine, furan, thiophene, and 1,2,5 thiadiazole. In another class of this embodiment, cycloalkyl is selected from the group consisting of: cyclobutyl, cyclopropyl, cyclopentyl and cyclohexyl.

In another embodiment of the present invention, R² is selected from the group consisting of: hydrogen, —C₁₋₈alkyl, —C₂₋₈alkenyl, —C₂₋₈alkynyl, —(CH₂)ₙC₃₋₇cycloalkyl, —(CH₂)ₙC₂₋₉heterocycloalkyl, —(CH₂)ₙphenyl, —(CH₂)ₙnaphthyl, —(CH₂)ₙheteroaryl, wherein alkyl, alkene, alkynyl, cycloalkyl, heterocycloalkyl, aryl, phenyl, naphthyl, heteroaryl, and (CH₂) are unsubstituted or substituted with one to four substituents independently selected from R⁷, and wherein two C₁₋₄ alkyl substituents on the same (CH₂) carbon may cyclize to form a 3- to 6-membered ring, provided that when X is a bond or —(CH₂)ₘ then R² is not hydrogen, —C₁₋₈alkyl, —C₂₋₈alkenyl, —C₂₋₈alkynyl, —(CH₂)ₙC₃₋₇cycloalkyl, —C₂₋₉heterocycloalkyl, -phenyl, -benzyl, -naphthyl, -heteroaryl, —OR⁶, —C(O)R⁶, or —S—C₁₋₈alkyl, further provided that when X is a bond R² is not —NH₂, —CO₂C₁₋₈alkyl, —CO₂C₃₋₇cycloalkyl, —CO₂(CH₂)₀₋₁phenyl, and provided that when X is —(CH₂)ₘNR⁶— then R² is not —C(O)R⁶.

In another class of this embodiment, when X is NR⁶(CH₂)ₙ and R² is heterocycloalkyl or heteroaryl, then heterocycloalkyl is selected from the group consisting of: piperidine, pyrrolidine, octahydropyrrolopyrazine, and 2-aza[2.2.1]bicycloheptane, and heteroaryl is selected from the group consisting of: isoindoline, tetrahydroisoquinoline, 3,4 dihydro-2H-isoquinolin-1-one, tetrahydroisoquinolin-1-one, octahydropyrrolopyrazine, dihydrobenzothiazole, and dihydrobenzoxazole. In another class of this embodiment, when X is NR⁶(CH₂)ₙNR⁶— and R² is —C(O)(CH₂)ₙheteroaryl, —C(O)(CH₂)ₙheterocycloalkyl or C(O)cycloalkyl, then heteroaryl is selected from the group consisting of: pyrrole, furan, imidazole, oxazole, pyridine, pyrimidine, triazole, tetrazole, piperazine, pyrazole, thiophene, oxadiazole, thiazole, thiadiazole, indole, triazolopyrimidine, pyrazolopyrimidine, 1,3-benzodioxole, isoxazole, benzothiazole, benzimidazole, benzoxadiazole, benzothiadiazole, benzotriazole, benzofuran, benzodioxane, benzothiophene, dihyrobenzofuran, quinoline, quinoxaline, imidazopyridine and pyrazolopyridine; heterocycloalkyl is selected from the group consisting of: 1,3-dihydroimidazol-2-one, 2,4-dihydro[1,2,4]triazol-3-one, pyrrolidine and piperidine; and cycloalkyl is selected from the group consisting of: cyclobutyl and cyclohexyl. In another class of this embodiment, when X is NR⁶(CH₂)ₙNR⁶— and R² is —(CH₂)ₙheteroaryl, —(CH₂)ₙheterocycloalkyl or —(CH₂)ₙaryl, then heteroaryl is selected from the group consisting of: —CH₂pyridine, pyridine, —CH₂pyrimidine, —CH₂indole, benzothiazole, benzoxazole, —CH₂pyrazine, —CH₂benzimidazole, —CH₂quinoline, —CH₂pyrazopyridine, and benzimidazole; heterocycloalkyl is selected from the group consisting of: —CH₂ dioxane, piperidine, morpholine, and tetrahydropyran; and aryl is selected from the group consisting of: phenyl and naphthalene. In another class of this embodiment, when X is —NR⁶(CH₂)ₙheteroaryl, and R² is —(CH₂)ₙaryl, then heteroaryl is -oxadiazole, and aryl is phenyl and naphthalene. In another class of this embodiment, when X is —NR⁶(CH₂)ₙheterocycloalkyl- and R² is —C(O)heteroaryl, then heterocycloalkyl is selected from: pyrrolidine and piperazine; and heteroaryl is thiophene. In another class of this embodiment, when X is —NR⁶(CH₂)ₙheterocycloalkyl- and R² is —(CH₂)heteroaryl, then heterocycloalkyl is selected from: pyrrolidine and piperazine; and heteroaryl is —CH₂indole. In another class of this embodiment, when X is —(CH₂)ₙheterocycloalkyl- and R² is —C(O)heteroaryl, then heterocycloalkyl is selected from: pyrrolidine, piperidine, and piperazine; and heteroaryl is indole. In another class of this embodiment, when X is —(CH₂)ₙheterocycloalkyl-NR⁶— and R² is —C(O)heteroaryl, then heterocycloalkyl is pyrrolidine; and heteroaryl is indole. In another class of this embodiment, when X is —NR⁶-phenyl-NR⁶— and R² is —C(O)heteroaryl, then phenyl is meta or para substituted and heteroaryl is indole or benzimidazole. In another class of this embodiment, X is —NR⁶(CH₂)ₙheterocycloalkyl-, wherein heterocycloalkyl is piperidine.

In another embodiment of the compounds of structural formula I, R³ is selected from the group consisting of: —C(O)C₁₋₈alkyl, —CO₂R⁵, —C(O)N(R⁶)OC₁₋₈alkyl, —C(O)C₁₋₄alkenylphenyl, —C(O)C₁₋₄alkynylphenyl, —C(O)phenyl, —C(O)naphthyl, —C(O)heteroaryl, and —C(O)C₃₋₇cycloalkyl, wherein alkyl, alkenyl, phenyl, naphthyl, heteroaryl, and cycloalkyl are unsubstituted or substituted with one to three groups independently selected from R⁸. In a class of this embodiment, R³ is selected from the group consisting of: —CO₂R⁵, —C(O)N(R⁶)OC₁₋₈alkyl, —C(O)phenyl, and —C(O)heteroaryl, wherein phenyl, and heteroaryl are unsubstituted or substituted with one to three groups independently selected from R⁸. In a subclass of this class, R³ is selected from the group consisting of: —C(O)N(CH₃)OCH₃, —C(O)phenyl, and —C(O)-(1,3-benzodioxole), wherein phenyl is substituted with 1-3 substituents selected from: CF₃, Br and CH₃. In a subclass of this subclass, R³ is —C(O)phenyl, wherein phenyl is substituted with 1-3 substituents selected from: CF₃, Br and CH₃. In another subclass of this subclass, R³ is —C(O)phenyl, wherein phenyl is substituted with CH₃.

In another embodiment of this invention, R³ is selected from the group consisting of: —C₁₋₈alkyl, —(CH₂)ₙ-phenyl, —(CH₂)ₙ-naphthyl, and —(CH₂)ₙC₃₋₇cycloalkyl, wherein alkyl, alkenyl, phenyl, naphthyl, heteroaryl, and cycloalkyl are unsubstituted or substituted with one to three groups independently selected from R⁸, and each (CH₂)ₙ is unsubstituted or substituted with 1 to 2 groups independently selected from: C₁₋₄alkyl, —OH, halogen, and C₁₋₄ alkenyl.

In another embodiment of the compounds of structural formula I, R⁴ is selected from the group consisting of: phenyl, naphthyl, and heteroaryl, wherein phenyl, naphthyl, heteroaryl, and (CH₂) are unsubstituted or substituted with one to three groups independently selected from halogen, —C₁₋₆alkyl, —C₂₋₆alkenyl, —C₂₋₆alkynyl, phenyl, —CH₂phenyl, —(CH₂)ₙOR⁶, —CN, —OCF₃, —CF₃, —NO₂, —NR⁵COR⁵, —CO₂R⁵, and —CO₂H. In a class of this embodiment, R⁴ is phenyl, wherein phenyl is unsubstituted or substituted with one to three groups independently selected from halogen, —C₁₋₆alkyl, —C₂₋₆alkenyl, —C₂₋₆alkynyl, phenyl, —CH₂phenyl, —(CH₂)ₙOR⁶, —CN, —OCF₃, —CF₃, —NO₂, —NR⁵COR⁵, —CO₂R⁵, and —CO₂H. In a subclass of this class, R⁴ is phenyl, wherein phenyl is unsubstituted or substituted with one to three groups independently selected from chloride, fluoride and iodide. In a subclass of this class, R⁴ is phenyl, wherein phenyl is unsubstituted or para substituted with chloride or fluoride.

In another embodiment of the present invention, each R⁷ is independently selected from the group consisting of: halogen, oxo, ═NH, —CN, —CF₃, —C₁₋₆ alkyl, —(CH₂)ₙC₃₋₆cycloalkyl, —(CH₂)ₙC₂₋₉heterocycloalkyl, —(CH₂)ₙOR⁶, —(CH₂)ₙCO₂R⁶, —(CH₂)ₙphenyl, —(CH₂)ₙ—O-phenyl, —(CH₂)ₙ-heteroaryl, —N(R⁶)₂, —NR⁶C(O)R⁶, —SR⁵, —SO₂C₁₋₆alkyl, and —SO₂N(R⁶)₂, wherein alkyl, phenyl, heteroaryl, heterocycloalkyl, cycloalkyl, and (CH₂)ₙ are unsubstituted or substituted with one to three groups independently selected from oxo, halogen, C₁₋₄ alkyl and OR⁵. In a class of this embodiment, each R⁷ is independently selected from the group consisting of: Br, I, F, Cl, oxo, ═NH, —CN, —CF₃, —CH₃, —CH₂CH₃, —CH(CH₃)₂, —C(CH₃)₃, cyclopropyl, succinamide, —CH₂OCH₃, —CH₂OH, —OCH₃, —OCH₂CH₃, —O(CH₂)₃CH₃, —OCH(CH₃)₂, —CO₂CH₃, —CO₂H, -phenyl, —CH₂-phenyl, —O-phenyl, pyridine, pyrazole, tetrazole, —N(CH₃)₂, —NH₂, —NHC(O)CH₃, —SCH₃, —SO₂CH₃, and —SO₂NH₂, wherein the R7 substituents are unsubstituted or substituted with one to three groups independently selected from oxo, halogen, C₁₋₄ alkyl and OR⁵.

In another embodiment of the compounds of structural formula I, there are provided compounds of structural formula II:

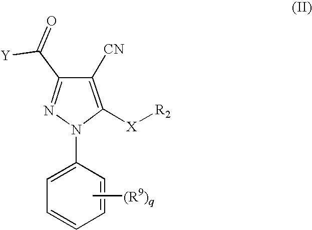

wherein Y is selected from the group consisting of: —C₁₋₈alkyl, —OR⁵, —N(R⁶)OC1-8alkyl, —C₁₋₄alkenylphenyl, —C₁₋₄alkynylphenyl, -phenyl, -naphthyl, -heteroaryl, and —C₃₋₇cycloalkyl, wherein alkyl, alkenyl, alkynyl, phenyl, naphthyl, heteroaryl, and cycloalkyl are unsubstituted or substituted with one to three groups independently selected from R⁸, and X, R², R⁸ and R⁹ are as defined above, p is 0 to 3 and q is 0 to 3; or a pharmaceutically acceptable salt thereof.

In another embodiment of the compounds of structural formula I, there are provided compounds of structural formula III:

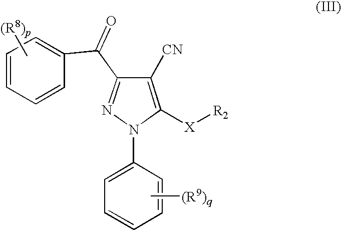

wherein X, R², R⁸ and R⁹ are as defined above, p is 0 to 3 and q is 0 to 3; or a pharmaceutically acceptable salt thereof. In a class of this embodiment, q is 1 and R⁹ is halogen. In another class of this embodiment, p is 1 and R⁸ is methyl.

In another embodiment of the compounds of structural formula I, there are provided compounds of structural formula IV:

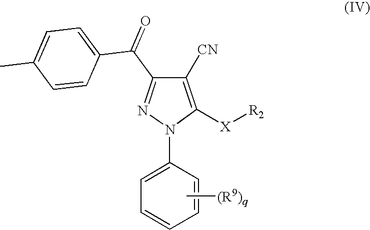

wherein X and R² are as defined above, q is 0 to 3, and R⁹ is independently selected from the group consisting of: halogen, —C₁₋₆alkyl, —C₂₋₆alkenyl, —C₂₋₆alkynyl, phenyl, —CH₂-phenyl, —(CH₂)ₙOR⁶, —CN, —OCF₃, —CF₃, —NO₂, —NR⁵COR⁵, —CO₂R⁵, and —CO₂H; or a pharmaceutically acceptable salt thereof. In a class of this embodiment, R⁹ is halogen.

Illustrative but nonlimiting examples of compounds of the present invention that are useful as ghrelin antagonists/inverse agonists are the following:

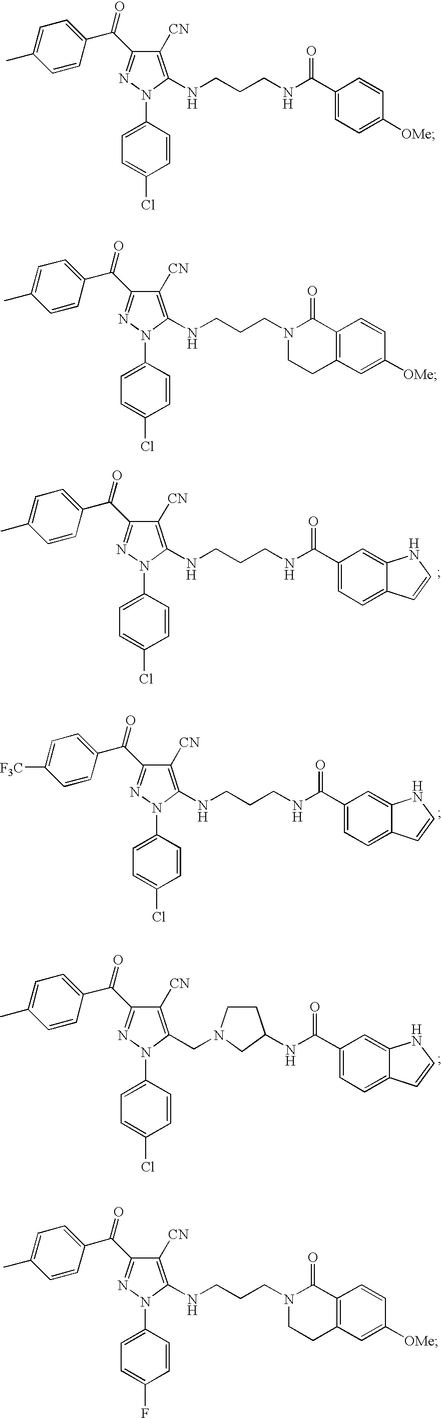

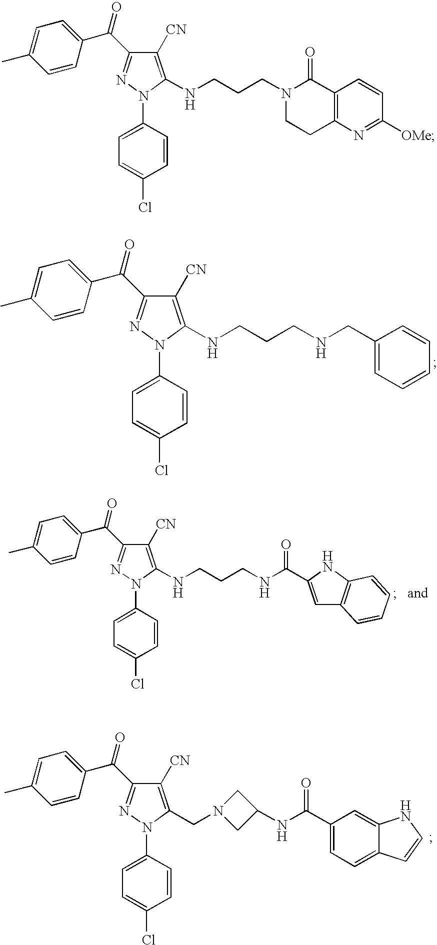

or a pharmaceutically acceptable salt thereof.

The compounds of structural formula I, II, III and IV are effective as ghrelin receptor antagonists/inverse agonists and are particularly effective as antagonists/inverse agonists of the ghrelin receptor. They are therefore useful for the treatment and/or prevention of disorders responsive to the modulation of the ghrelin receptor, such as obesity, diabetes, obesity-related disorders, and metabolic syndrome.

Another aspect of the present invention provides a method for the treatment or prevention of obesity, diabetes, metabolic syndrome, or an obesity-related disorder in a subject in need thereof which comprises administering to said subject a therapeutically or prophylactically effective amount of a ghrelin receptor antagonist/inverse agonist of the present invention. The present invention also relates to methods for treating or preventing obesity by administering a ghrelin antagonist/inverse agonist of the present invention in combination with a therapeutically or prophylactically effective amount of another agent known to be useful to treat or prevent the condition. The present invention also relates to methods for treating or preventing diabetes, metabolic syndrome or an obesity-related disorder by administering a ghrelin receptor antagonist/inverse agonist of the present invention in combination with a therapeutically or prophylactically effective amount of another agent known to be useful to treat or prevent the condition.

Another aspect of the present invention provides a pharmaceutical composition comprising a compound of structural formula I, II, III or IV, and a pharmaceutically acceptable carrier.

Yet another aspect of the present invention relates to the use of a compound of structural formula I, II, III or IV for the manufacture of a medicament useful for the treatment or prevention, or suppression of a disease mediated by the ghrelin receptor in a subject in need thereof.

Yet another aspect of the present invention relates to the use of a ghrelin antagonist/inverse agonist of the present invention for the manufacture of a medicament useful for the treatment or prevention, or suppression of a disease mediated by the ghrelin receptor, wherein the disease is selected from the group consisting of obesity, diabetes, metabolic syndrome and an obesity-related disorder in a subject in need thereof. Another aspect of the present invention relates to the use of a ghrelin antagonist/inverse agonist of the present invention for the manufacture of a medicament useful for the treatment or prevention of obesity in a subject in need thereof. Another aspect of the present invention relates to the use of a ghrelin antagonist/inverse agonist of the present invention for the manufacture of a medicament useful for the treatment or prevention of diabetes in a subject in need thereof. Another aspect of the present invention relates to the use of a ghrelin antagonist/inverse agonist of the present invention for the manufacture of a medicament useful for the treatment or prevention of metabolic syndrome in a subject in need thereof.

Yet another aspect of the present invention relates to the use of a therapeutically effective amount of a ghrelin receptor antagonist/inverse agonist of formula I, II, III or IV, or a pharmaceutically acceptable salt thereof, and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, and pharmaceutically acceptable salts thereof, for the manufacture of a medicament useful for the treatment, control, or prevention of obesity, diabetes, metabolic syndrome, or an obesity-related disorder in a subject in need of such treatment. Yet another aspect of the present invention relates to the use of a therapeutically effective amount of a ghrelin receptor antagonist/inverse agonist of formula I, II, III or IV, and pharmaceutically acceptable salts and esters thereof, and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, and pharmaceutically acceptable salts thereof, for the manufacture of a medicament for treatment or prevention of obesity, diabetes, metabolic syndrome, or an obesity-related disorder which comprises an effective amount of a ghrelin receptor antagonist/inverse agonist of formula I, II, III or IV and an effective amount of the agent, together or separately. Yet another aspect of the present invention relates to a product containing a therapeutically effective amount of a ghrelin receptor antagonist/inverse agonist of formula I, II, III, or IV, or a pharmaceutically acceptable salt thereof; and a therapeutically effective amount of an agent selected from the group consisting of an insulin sensitizer, an insulin mimetic, a sulfonylurea, an α-glucosidase inhibitor, a HMG-CoA reductase inhibitor, a serotonergic agent, a β3-adrenoreceptor agonist, a neuropeptide Y1 antagonist, a neuropeptide Y2 agonist, a neuropeptide Y5 antagonist, a pancreatic lipase inhibitor, a cannabinoid CB₁ receptor antagonist or inverse agonist, a melanin-concentrating hormone receptor antagonist, a bombesin receptor subtype 3 agonist, a ghrelin receptor antagonist, and a NK-1 antagonist, and pharmaceutically acceptable salts thereof, as a combined preparation for simultaneous, separate or sequential use in obesity, diabetes, or an obesity-related disorder.

Ghrelin receptor antagonist/inverse agonist compounds can be provided in kit. Such a kit typically contains an active compound of formula I, II, III or IV in dosage forms for administration. A dosage form contains a sufficient amount of active compound such that a beneficial effect can be obtained when administered to a patient during regular intervals, such as 1, 2, 3, 4, 5 or 6 times a day, during the course of 1 or more days. Preferably, a kit contains instructions indicating the use of the dosage form for weight reduction (e.g., to treat obesity) and the amount of dosage form to be taken over a specified time period.

Throughout the instant application, the following terms have the indicated meanings:

The term “alkyl”, as well as other groups having the prefix “alk”, such as alkoxy, alkanoyl, means carbon chains of the designated length which may be in a straight or branched configuration, or combinations thereof. The term alkyl also includes methylene groups which are designated as (CH₂) herein. Examples of alkyl groups include methyl, ethyl, n-propyl, isopropyl, n-butyl, 1-methylpropyl, 2-methylpropyl, tert-butyl, n-pentyl, 1-methylbutyl, 2-methylbutyl, 3-methylbutyl, 1,2-dimethylpropyl, 1,1-dimethylpropyl, 2,2-dimethylpropyl, n-hexyl, 1-methylpentyl, 2-methylpentyl, 3-methylpentyl, 4-methylpentyl, 1-ethylbutyl, 2-ethylbutyl, 3-ethylbutyl, 1,1-dimethyl butyl, 1,2-dimethylbutyl, 1,3-dimethylbutyl, 2,2-dimethylbutyl, 2,3-dimethylbutyl, 3,3-dimethyl butyl, n-heptyl, 1-methylhexyl, 2-methylhexyl, 3-methylhexyl, 4-methylhexyl, 5-methylhexyl, 1-ethylpentyl, 2-ethylpentyl, 3-ethylpentyl, 4-ethylpentyl, 1-propylbutyl, 2-propylbutyl, 3-propylbutyl, 1,1-dimethylpentyl, 1,2-dimethylpentyl, 1,3-dimethylpentyl, 1,4-dimethylpentyl, 2,2-dimethylpentyl, 2,3-dimethylpentyl. 2,4-dimethylpentyl, 3,3-dimethylpentyl, 3,4-dimethylpentyl, 4,4-dimethylpentyl, 1-methyl-1-ethylbutyl, 1-methyl-2-ethylbutyl, 2-methyl-2-ethylbutyl, 1-ethyl-2-methylbutyl, 1-ethyl-3-methylbutyl, 1,1-diethylpropyl, n-octyl, n-nonyl, and the like.

The term “halogen” is intended to include the halogen atoms fluorine, chlorine, bromine and iodine; preferably fluorine, chlorine and bromine.

The terms “alkene”, “alkenyl” and “C₂₋₈alkenyl” means a two to eight carbon chain with at least one double bond.

The terms “alkyne”, “alkynyl” and “C₂₋₈alkynyl” means a two to eight carbon chain with at least one triple bond.

The term “aryl” includes phenyl, naphthalene and indan.

The term “heteroaryl” includes three to ten carbon mono- and bicyclic aromatic rings containing from 1 to 4 heteroatoms selected from nitrogen, oxygen and sulfur, wherein at least one of the rings of the bicyclic ring system is aromatic. Substitution on the heteroaryl ring includes mono substitution on any carbon, sulfur and nitrogen of the heteroaryl ring. Examples of heteroaryls include, but are not limited to, furan, thiene, thiaphene, thiophene, pyrrole, isoxazole, oxazole, thiazole, triazole, 1,2,4 triazole, triazine, tetrazole, thiadiazole, 1,2,5 thiadiazole, imidazole, isoxazole, isothiazole, naphthene, oxadiazole, 1,2,5 oxadiazole, 1,2,4 oxadiazole, 1,2,5 oxadiazole, pyrazole, pyridine, pyrimidine, pyrazine, pyridazine, quinole, isoquinole, benzimidazole, benzofuran, benzothiene, indole, benzthiazole, benzoxazole, and the like. Bicyclic heteroaromatic ring includes, but are not limited to, 1,3 benzodioxole, 1,4 benzodioxan, benzothiadiazole, indole, benzothiaphene, benzo(b)thiophene, benzo(c)thiophene, benzofuran, 1,4-benzofurazan, benzimidazole, benzisoxazole, benzothiazole, benzotriazole, benzoxazole, isoquinoline, purine, furopyridine, thienopyridine, benzisodiazole, indoline, indole, phthalimide, benzyl(1,2,3,4)tetrahydroisoquinoline, triazolopyrimidine; 5,6,7,8-tetrahydroquinoline, quinoline, quinazoline, 2,3-dihydro-benzofuran, imidazo[1,2-a]pyridine, quinoxaline, [1,2,4]triazolo[1,5-a]pyrimidine, 1H-pyrazolo[4,3-b]pyridine, 1,3-dihydro-benzo[1,2,5]oxadiazole, 1,3-dihydro-benzo[1,2,5]thiadiazole, benzo[1,2,3]thiadiazole, 2,3-dihydro-benzo[b]thiophene, 1,2,3,4-tetrahydro-isoquinoline, 1,3-dihydro-imidazo[4,5-b]pyrazin-2-one, 3H-benzothiazol-2-ylideneamine, 5,6,7,8-tetrahydro-imidazo[1,2-a]pyrazine, benzo[b]thiophene, pyrazolo[1,5-a]pyridine, 3,4-dihydro-2H-sioquinolin-1-one, quinoxaline, pyridazine, dihydrobenzoxazole and dihydrobenzothiazole. Bicyclic heteroaromatic or bicyclic heteroaryl rings include, but are not limited to, two aromatic rings fused together, as well as one aromatic fuse to a non aromatic ring.

The term “cycloalkyl” includes mono- or bicyclic non-aromatic rings, containing only carbon atoms, which may contain double bonds. Examples of cycloalkyl include, but are not limited to, cyclopropane, cyclobutane, cyclopentane, cyclopentene, cyclohexene, cyclohexane, and cycloheptane.

The term “heterocycloalkyl” includes two to ten carbon mono- or bicyclic non-aromatic heterocycles containing one to four heteroatoms selected from nitrogen, oxygen, sulfur, sulfone, and sulfoxide. Substitution on the heterocycloalkyl ring includes mono- or di-substitution on any carbon and/or monosubstitution on any nitrogen of the heterocycloalkyl ring. Examples of heterocycloalkyls include, but are not limited to, azetidine, piperidine, piperazine, morpholine, thiamorpholine, tetrahydropyran, 1,4-tetrahydropyran, thiatetrahydropyran, pyrrolidine, imidazolidine, tetrahydrofuran, 1-thia-4-aza-cyclohexane, 2-azabicyclo[2.2.1]heptane, succinimide, 1,3-dioxane, 1,3-dihydroimidazol-2-one, 2,4-dihydro-[1,2,4]triazol-3-one, and octahydro-pyrrolo[1,2-a]pyrazine.

Certain of the above defined terms may occur more than once in the above formula and upon such occurrence each term shall be defined independently of the other; thus for example, NR⁶R⁶ may represent NH₂, NHCH₃, N(CH₃)CH₂CH₃, and the like.

The term “subject” means a mammal. One embodiment of the term “mammal” is a “human,” said human being either male or female. The instant compounds are also useful for treating or preventing obesity and obesity related disorders in cats and dogs. As such, the term “mammal” includes companion animals such as cats and dogs. The term “mammal in need thereof” refers to a mammal who is in need of treatment or prophylaxis as determined by a researcher, veterinarian, medical doctor or other clinician.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

By a ghrelin receptor “antagonist” or “inverse agonist” is meant a drug or a compound that blocks the ghrelin receptor-associated responses normally induced by a bioactive ghrelin receptor agonist, while an inverse agonist has the additional property of inhibiting the ligand independent activity associated with the ghrelin receptor (see e.g. Holst-B; Cygankiewicz-A; Halkjaer-T; Ankersen-M; Schwartz-T; Mol-Endocrinol. 2003; 17(11): 2201-2210). The “antagonistic” or “inverse agonistic” properties of the compounds of the present invention were measured as IC₅₀ values in the functional assay described below. The functional assay discriminates a ghrelin receptor antagonist or ghrelin receptor inverse agonist from a ghrelin receptor agonist; antagonists display an antagonistic efficacy (inhibition) between 0% and 100% inhibition, while inverse agonists displayed an antagonist efficacy of greater than 100% inhibition.

By “inverse agonist” is meant a compound that decreases the basal functional activity of the ghrelin receptor. Inverse agonism is a property of the ligand alone on the receptor. The term also includes partial inverse agonists, which only decrease the basal activity of the receptor to a certain level, but not fully. Certain compounds may be both inverse agonists (in the absence of hormone) and antagonists (in the presence of hormone).

By “antagonist” is meant a compound that decreases the functional activity of a biological target molecule by inhibiting the action of an agonist (for example ghrelin). Antagonism is a property of the ligand measured in the presence of a compound with higher signaling efficacy (usually a full agonist).

By “basal activity”, “basal functional activity” or “basal signaling activity” of the ghrelin receptor is meant the signaling activity of the receptor in the absence of any ligand, i.e. hormone.

By “binding affinity” is meant the ability of a compound/drug to bind to its biological target, in the present instance, the ability of a compound of structural formula I, II, III or IV to bind to a ghrelin receptor. Binding affinities for the compounds of the present invention were measured in the binding assay described below and are expressed as IC₅₀'s.

By the term “selective” or “selective ghrelin receptor antagonist” or “selective ghrelin receptor inverse agonist” is meant a compound that binds selectively to the ghrelin or growth hormone secretagogue receptor and not to other unrelated G protein coupled receptors.

“Efficacy” describes the relative intensity with which antagonists or inverse agonists vary in the response they produce even when they occupy the same number of receptors and with the same affinity. Efficacy is the property that enables compounds to produce responses. Properties of compounds can be categorized into two groups, those which cause them to associate with the receptors (binding affinity) and those that produce a stimulus (efficacy). The term “efficacy” is used to characterize the level of maximal responses induced by antagonists or inverse agonists. Not all antagonists or inverse agonists of a receptor are capable of inducing identical levels of maximal responses. Maximal response depends on the efficiency of receptor coupling, that is, from the cascade of events, which, from the binding of the drug to the receptor, leads to the desired biological effect.

The functional activities expressed as IC₅₀'s and the “antagonist efficacy” or “inverse agonist efficacy” for the compounds of the present invention at a particular concentration were measured in the functional assay described below.

Compounds of structural formula I, II, III and IV contain one or more asymmetric centers and can thus occur as rotamers, racemates and racemic mixtures, single enantiomers, diastereomeric mixtures and individual diastereomers. The present invention is meant to comprehend all such isomeric forms of the compounds of structural formula I, II, III and IV, including the E and Z geometric isomers of olefinic double bonds. Some of the compounds described herein may exist as tautomers such as keto-enol tautomers. The individual tautomers as well as mixtures thereof are encompassed within the compounds of structural formula I, II, III and IV.

Compounds of structural formula I, II, III and IV may be separated into their individual diastereoisomers by, for example, fractional crystallization from a suitable solvent, for example methanol or ethyl acetate or a mixture thereof, or via chiral chromatography using an optically active stationary phase. Absolute stereochemistry may be determined by X-ray crystallography of crystalline products or crystalline intermediates which are derivatized, if necessary, with a reagent containing an asymmetric center of known absolute configuration.

Alternatively, any stereoisomer of a compound of the general formula I, II, II and IV may be obtained by stereospecific synthesis using optically pure starting materials or reagents of known absolute configuration.

It will be understood that the compounds of the present invention include hydrates, solvates, polymorphs, crystalline, hydrated crystalline and amorphous forms of the compounds of the present invention, and pharmaceutically acceptable salts thereof.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Particularly preferred are citric, fumaric, hydrobromic, hydrochloric, maleic, phosphoric, sulfuric, and tartaric acids.

It will be understood that, as used herein, references to the compounds of formula I, II, III and IV are meant to also include the pharmaceutically acceptable salts, such as the hydrochloride salts.

The compounds of formula I, II, III and IV are ghrelin receptor ligands and as such are useful in the treatment, control or prevention of diseases, disorders or conditions responsive to the modulation of the ghrelin receptor. In particular, the compounds of formula I, II, III and IV act as ghrelin receptor antagonists/inverse agonists useful in the treatment, control or prevention of diseases, disorders or conditions responsive to the blockade of the ghrelin receptor.

Such diseases, disorders or conditions include, but are not limited to, obesity (including inducing weight loss, reducing bodyweight, reducing food intake, reducing appetite, increasing metabolic rate, reducing fat intake, reducing carbohydrate craving; or inducing satiety), diabetes mellitus (including enhancing glucose tolerance, and/or decreasing insulin resistance), type II diabetes, hypertension, hyperlipidemia, osteoarthritis, cancer, gall bladder disease, sleep apnea, depression, anxiety, compulsion, neuroses, insomnia/sleep disorder, substance abuse, pain, male and female sexual dysfunction (including male impotence, loss of libido, female sexual arousal dysfunction, female orgasmic dysfunction, hypoactive sexual desire disorder, sexual pain disorder and male erectile dysfunction), fever, inflammation, immune modulation, rheumatoid arthritis, neuroprotective and cognitive and memory enhancement including the treatment of Alzheimer's disease, and obesity related disorders.

Antagonists/inverse agonists encompassed by formula I, II, III and IV show a high affinity for the ghrelin receptor, which makes them especially useful in the prevention and treatment of obesity, diabetes, metabolic syndrome, metabolic disorders, and obesity-related disorders.

The compositions of the present invention are useful for the treatment or prevention of disorders associated with excessive food intake, such as obesity and obesity-related disorders. The obesity herein may be due to any cause, whether genetic or environmental.

The obesity-related disorders herein are associated with, caused by, or result from obesity. Examples of obesity-related disorders include overeating, binge eating, and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome, Frohlich's syndrome, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer, nicotine addiction, substance addiction and alcoholism. The compositions of the present invention are also useful for reducing the risk of secondary outcomes of obesity, such as reducing the risk of left ventricular hypertrophy.

The term “metabolic syndrome”, also known as syndrome X, is defined in the Third Report of the National Cholesterol Education Program Expert Panel on Detection, Evaluation and Treatment of High Blood Cholesterol in Adults (ATP-III). E. S. Ford et al., JAMA, vol. 287 (3), Jan. 16, 2002, pp 356-359. Briefly, a person is defined as having metabolic syndrome if the person has three or more of the following symptoms: abdominal obesity, hypertriglyceridemia, low HDL cholesterol, high blood pressure, and high fasting plasma glucose. The criteria for these are defined in ATP-III.

The term “diabetes,” as used herein, includes both insulin-dependent diabetes mellitus (i.e., IDDM, also known as type I diabetes) and non-insulin-dependent diabetes mellitus (i.e., NIDDM, also known as Type II diabetes). Type I diabetes, or insulin-dependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compositions of the present invention are useful for treating both Type I and Type II diabetes. The compositions are especially effective for treating Type II diabetes. The compounds or combinations of the present invention are also useful for treating and/or preventing gestational diabetes mellitus.

Treatment of diabetes mellitus refers to the administration of a compound or combination of the present invention to treat diabetes. One outcome of treatment may be decreasing the glucose level in a subject with elevated glucose levels. Another outcome of treatment may be improving glycemic control. Another outcome of treatment may be decreasing insulin levels in a subject with elevated insulin levels. Another outcome of treatment may be decreasing plasma triglycerides in a subject with elevated plasma triglycerides. Another outcome of treatment may be lowering LDL cholesterol in a subject with high LDL cholesterol levels. Another outcome of treatment may be increasing HDL cholesterol in a subject with low HDL cholesterol levels. Another outcome may be decreasing the LDL/HDL ratio in a subject in need thereof. Another outcome of treatment may be increasing insulin sensitivity. Another outcome of treatment may be enhancing glucose tolerance in a subject with glucose intolerance. Another outcome of treatment may be decreasing insulin resistance in a subject with increased insulin resistance or elevated levels of insulin. Another outcome may be decreasing triglycerides in a subject with elevated triglycerides. Yet another outcome may be improving LDL cholesterol, non-HDL cholesterol, triglyceride, HDL cholesterol or other lipid analyte profiles.

Prevention of diabetes mellitus refers to the administration of a compound or combination of the present invention to prevent the onset of diabetes in a subject at risk thereof.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility. In particular, co-morbidities include: hypertension, hyperlipidemia, dyslipidemia, glucose intolerance, cardiovascular disease, sleep apnea, diabetes mellitus, and other obesity-related conditions.

Treatment of obesity and obesity-related disorders refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in subjects in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

Prevention of obesity and obesity-related disorders refers to the administration of the compounds or combinations of the present invention to reduce or maintain the body weight of a subject at risk of obesity. One outcome of prevention may be reducing the body weight of a subject at risk of obesity relative to that subject's body weight immediately before the administration of the compounds or combinations of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovary disease, cardiovascular diseases, osteoarthritis, dermatological disorders, hypertension, insulin resistance, hypercholesterolemia, hypertriglyceridemia, and cholelithiasis.

The terms “administration of” and or “administering” a compound should be understood to mean providing a compound of the invention or a prodrug of a compound of the invention to a subject in need of treatment. The administration of the compounds of the present invention in order to practice the present methods of therapy is carried out by administering a therapeutically effective amount of the compound to a subject in need of such treatment or prophylaxis. The need for a prophylactic administration according to the methods of the present invention is determined via the use of well known risk factors.

The term “therapeutically effective amount” as used herein means the amount of the active compound that will elicit the biological or medical response in a tissue, system, subject, mammal, or human that is being sought by the researcher, veterinarian, medical doctor or other clinician, which includes alleviation of the symptoms of the disorder being treated. The novel methods of treatment of this invention are for disorders known to those skilled in the art. The term “prophylactically effective amount” as used herein means the amount of the active compound that will elicit the biological or medical response in a tissue, system, subject, mammal, or human that is being sought by the researcher, veterinarian, medical doctor or other clinician, to prevent the onset of the disorder in subjects as risk for obesity or the disorder. The therapeutically or prophylactically effective amount, or dosage, of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgement.

### Administration and Dose Ranges

Any suitable route of administration may be employed for providing a subject or mammal, especially a human with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of formula I, II, III and IV are administered orally or topically.

The effective dosage of active ingredient employed may vary depending on the particular compound employed, the mode of administration, the condition being treated and the severity of the condition being treated. Such dosage may be ascertained readily by a person skilled in the art.

When treating obesity generally satisfactory results are obtained when the compounds of formula I, II, III and IV are administered at a daily dosage of from about 0.001 milligram to about 50 milligrams per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating diabetes mellitus and/or hyperglycemia, as well as other diseases or disorders for which compounds of formula I, II, III and IV are useful, generally satisfactory results are obtained when the compounds of the present invention are administered at a daily dosage of from about 0.001 milligram to about 50 milligram per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

When treating metabolic syndrome or an obesity-related disorder generally satisfactory results are obtained when the compounds of formula I, II, III and IV are administered at a daily dosage of from about 0.001 milligram to about 50 milligrams per kilogram of animal body weight, preferably given in a single dose or in divided doses two to six times a day, or in sustained release form. In the case of a 70 kg adult human, the total daily dose will generally be from about 0.07 milligrams to about 3500 milligrams. This dosage regimen may be adjusted to provide the optimal therapeutic response.

In the case where an oral composition is employed, a suitable dosage range is, e.g. from about 0.01 mg to about 1500 mg of a compound of formula I, II, III and IV per day, preferably from about 0.1 mg to about 600 mg per day, more preferably from about 0.1 mg to about 100 mg per day. For oral administration, the compositions are preferably provided in the form of tablets containing from 0.01 to 1,000 mg, preferably 0.01, 0.05, 0.1, 0.5, 1, 2.5, 5, 10, 15, 20, 25, 30, 40, 50, 100, 250, 500, 600, 750, 1000, 1250 or 1500 milligrams of the active ingredient for the symptomatic adjustment of the dosage to the patient to be treated.

For use where a composition for intranasal administration is employed, intranasal formulations for intranasal administration comprising 0.001-10% by weight solutions or suspensions of the compounds of formula I, II, III and IV in an acceptable intranasal formulation may be used.

For use where a composition for intravenous administration is employed, a suitable dosage range is from about 0.001 mg to about 50 mg, preferably from 0.01 mg to about 50 mg, more preferably 0.1 mg to 10 mg, of a compound of formula I, II, III and IV per kg of body weight per day. This dosage regimen may be adjusted to provide the optimal therapeutic response. It may be necessary to use dosages outside these limits in some cases.

For the treatment of diseases of the eye, ophthalmic preparations for ocular administration comprising 0.001-1% by weight solutions or suspensions of the compounds of formula I, II, III and IV in an acceptable ophthalmic formulation may be used.

The magnitude of prophylactic or therapeutic dosage of the compounds of the present invention will, of course, vary depending on the particular compound employed, the mode of administration, the condition being treated and the severity of the condition being treated. It will also vary according to the age, weight and response of the individual patient. Such dosage may be ascertained readily by a person skilled in the art.

Compounds of formula I, II, III and IV may be used in combination with other drugs that are used in the treatment/prevention/suppression or amelioration of the diseases or conditions for which compounds of formula I, II, III and IV are useful. Such other drugs may be administered, by a route and in an amount commonly used therefor, contemporaneously or sequentially with a compound of formula I, II, III and IV. When a compound of formula I, II, III and IV is used contemporaneously with one or more other drugs, a pharmaceutical composition containing such other drugs in addition to the compound of formula I, II, III and IV is preferred. Accordingly, the pharmaceutical compositions of the present invention include those that also contain one or more other active ingredients, in addition to a compound of formula I, II, III, and IV.

Examples of other active ingredients that may be combined with a compound of formula I, II, III and IV for the treatment or prevention of obesity and/or diabetes and/or metabolic syndrome and/or an obesity-related disorder either administered separately or in the same pharmaceutical compositions, include, but are not limited to:

(a) insulin sensitizers including (i) PPARγ antagonists such as glitazones (e.g. ciglitazone; darglitazone; englitazone; isaglitazone (MCC-555); pioglitazone; rosiglitazone; troglitazone; tularik; BRL49653; CLX-0921; 5-BTZD), GW-0207, LG-100641, and LY-300512, and the like), and compounds disclosed in WO 97/10813, WO 97/27857, WO 97/28115, WO 97/28137, and WO 97/27847; (iii) biguanides such as metformin and phenformin;

(b) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulinotropin); and GLP-1 (7-36)-NH₂);

(c) sulfonylureas, such as acetohexamide; chlorpropamide; diabinese; glibenclamide; glipizide; glyburide; glimepiride; gliclazide; glipentide; gliquidone; glisolamide; tolazamide; and tolbutamide;

(d) α-glucosidase inhibitors, such as acarbose, adiposine; camiglibose; emiglitate; miglitol; voglibose; pradimicin-Q; salbostatin; CKD-711; MDL-25,637; MDL-73,945; and MOR 14, and the like;

(e) cholesterol lowering agents such as (i) HMG-CoA reductase inhibitors (atorvastatin, itavastatin, fluvastatin, lovastatin, pravastatin, rivastatin, rosuvastatin, simvastatin, and other statins), (ii) bile acid absorbers/sequestrants, such as cholestyramine, colestipol, dialkylaminoalkyl derivatives of a cross-linked dextran; Colestid®; LoCholest®, and the like, (ii) nicotinyl alcohol, nicotinic acid or a salt thereof, (iii) proliferator-activator receptor α agonists such as fenofibric acid derivatives (gemfibrozil, clofibrate, fenofibrate and benzafibrate), (iv) inhibitors of cholesterol absorption such as stanol esters, beta-sitosterol, sterol glycosides such as tiqueside; and azetidinones such as ezetimibe, and the like, and (acyl CoA:cholesterol acyltransferase (ACAT)) inhibitors such as avasimibe, and melinamide, (v) anti-oxidants, such as probucol, (vi) vitamin E, and (vii) thyromimetics;

(f) PPARα agonists such as beclofibrate, benzafibrate, ciprofibrate, clofibrate, etofibrate, fenofibrate, and gemfibrozil; and other fibric acid derivatives, such as Atromid®, Lopid® and Tricor®, and the like, and PPARα agonists as described in WO 97/36579 by Glaxo;

(g) PPARδ agonists, such as those disclosed in WO97/28149;

(h) PPAR α/δ agonists, such as muraglitazar, and the compounds disclosed in U.S. Pat. No. 6,414,002; and

(i) anti-obesity agents, such as (1) growth hormone secretagogues, growth hormone secretagogue receptor agonists/antagonists, such as NN703, hexarelin, MK-0677, SM-130686, CP-424,391, L-692,429, and L-163,255, and such as those disclosed in U.S. Pat. Nos. 5,536,716, and 6,358,951, U.S. Patent Application Nos. 2002/049196 and 2002/022637, and PCT Application Nos. WO 01/56592 and WO 02/32888; (2) protein tyrosine phosphatase-1B (PTP-1B) inhibitors; (3) cannabinoid receptor ligands, such as cannabinoid CB₁ receptor antagonists or inverse agonists, such as rimonabant (Sanofi Synthelabo), AMT-251, and SR-14778 and SR 141716A (Sanofi Synthelabo), SLV-319 (Solvay), BAY 65-2520 (Bayer), and those disclosed in U.S. Pat. Nos. 5,532,237, 4,973,587, 5,013,837, 5,081,122, 5,112,820, 5,292,736, 5,624,941, 6,028,084, PCT Application Nos. WO 96/33159, WO 98/33765, WO98/43636, WO98/43635, WO 01/09120, WO98/31227, WO98/41519, WO98/37061, WO00/10967, WO00/10968, WO97/29079, WO99/02499, WO 01/58869, WO 01/64632, WO 01/64633, WO 01/64634, WO02/076949, WO 03/007887, WO 04/048317, and WO 05/000809; and EPO Application No. EP-658546, EP-656354, EP-576357; (4) anti-obesity serotonergic agents, such as fenfluramine, dexfenfluramine, phentermine, and sibutramine; (5) β3-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, SR 59119A, and such as those disclosed in U.S. Pat. No. 5,705,515, and U.S. Pat. No. 5,451,677 and PCT Patent Publications WO94/18161, WO95/29159, WO97/46556, WO98/04526 and WO98/32753, WO 01/74782, and WO 02/32897; (6) pancreatic lipase inhibitors, such as orlistat (Xenical®), Triton WR1339, RHC80267, lipstatin, tetrahydrolipstatin, teasaponin, diethylumbelliferyl phosphate, and those disclosed in PCT Application No. WO 01/77094; (7) neuropeptide Y1 antagonists, such as BIBP3226, J-115814, BIBO 3304, LY-357897, CP-671906, GI-264879A, and those disclosed in U.S. Pat. No. 6,001,836, and PCT Patent Publication Nos. WO 96/14307, WO 01/23387, WO 99/51600, WO 01/85690, WO 01/85098, WO 01/85173, and WO 01/89528; (8) neuropeptide Y5 antagonists, such as GW-569180A, GW-594884A, GW-587081X, GW-548118X, FR226928, FR 240662, FR252384, 1229U91, GI-264879A, CGP71683A, LY-377897, PD-160170, SR-120562A, SR-120819A and JCF-104, and those disclosed in U.S. Pat. Nos. 6,057,335; 6,043,246; 6,140,354; 6,166,038; 6,180,653; 6,191,160; 6,313,298; 6,335,345; 6,337,332; 6,326,375; 6,329,395; 6,340,683; 6,388,077; 6,462,053; 6,649,624; and 6,723,847, hereby incorporated by reference in their entirety; European Patent Nos. EP-01010691, and EP-01044970; and PCT International Patent Publication Nos. WO 97/19682, WO 97/20820, WO 97/20821, WO 97/20822, WO 97/20823, WO 98/24768; WO 98/25907; WO 98/25908; WO 98/27063, WO 98/47505; WO 98/40356; WO 99/15516; WO 99/27965; WO 00/64880, WO 00/68197, WO 00/69849, WO 01/09120, WO 01/14376; WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/22592, WO 0248152, and WO 02/49648; WO 02/094825; WO 03/014083; WO 03/10191; WO 03/092889; WO 04/002986; and WO 04/031175; (9) melanin-concentrating hormone (MCH) receptor antagonists, such as those disclosed in WO 01/21577 and WO 01/21169; (10) melanin-concentrating hormone 1 receptor (MCH1R) antagonists, such as T-226296 (Takeda), and those disclosed in PCT Patent Application Nos. WO 01/82925, WO 01/87834, WO 02/051809, WO 02/06245, WO 02/076929, WO 02/076947, WO 02/04433, WO 02/51809, WO 02/083134, WO 02/094799, WO 03/004027, and Japanese Patent Application Nos. JP 13226269, and JP 2004-139909; (11) melanin-concentrating hormone 2 receptor (MCH2R) agonist/antagonists; (12) orexin-1 receptor antagonists, such as SB-334867-A, and those disclosed in PCT Patent Application Nos. WO 01/96302, WO 01/68609, WO 02/51232, and WO 02/51838; (13) serotonin reuptake inhibitors such as fluoxetine, paroxetine, and sertraline, and those disclosed in U.S. Pat. No. 6,365,633, and PCT Patent Application Nos. WO 01/27060 and WO 01/162341; (14) melanocortin agonists, such as Melanotan II or those described in WO 99/64002 and WO 00/74679; (15) Mc4r (melanocortin 4 receptor) agonists, such as CHIR86036 (Chiron), ME-10142, and ME-10145 (Melacure), CHIR86036 (Chiron); PT-141, and PT-14 (Palatin), and those disclosed in: U.S. Pat. Nos. 6,410,548; 6,294,534; 6,350,760; 6,458,790; 6,472,398; 6,376,509; and 6,818,658; US Patent Publication No. US2002/0137664; US2003/0236262; US2004/009751; US2004/0092501; and PCT Application Nos. WO 99/64002; WO 00/74679; WO 01/70708; WO 01/70337; WO 01/74844; WO 01/91752; WO 01/991752; WO 02/15909; WO 02/059095; WO 02/059107; WO 02/059108; WO 02/059117; WO 02/067869; WO 02/068387; WO 02/068388; WO 02/067869; WO 02/11715; WO 02/12166; WO 02/12178; WO 03/007949; WO 03/009847; WO 04/024720; WO 04/078716; WO 04/078717; WO 04/087159; WO 04/089307; and WO 05/009950; (16) 5HT-2 agonists; (17) 5HT2C (serotonin receptor 2C) agonists, such as BVT933, DPCA37215, WAY161503, R-1065, and those disclosed in U.S. Pat. No. 3,914,250, and PCT Application Nos. WO 02/36596, WO 02/48124, WO 02/10169, WO 01/66548, WO 02/44152, WO 02/51844, WO 02/40456, and WO 02/40457; (18) galanin antagonists; (19) CCK agonists; (20) CCK-A (cholecystokinin-A) agonists, such as AR-R 15849, GI 181771, JMV-180, A-71378, A-71623 and SR146131, and those described in U.S. Pat. No. 5,739,106; (21) GLP-1 agonists; (22) corticotropin-releasing hormone agonists; (23) histamine receptor-3 (H3) modulators; (24) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, GT2394 (Gliatech), and those described and disclosed in PCT Application No. WO 02/15905, and O-[3-(1H-imidazol-4-yl)propanol]-carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)); (25) β-hydroxy steroid dehydrogenase-1 inhibitors (β-HSD-1); 26) PDE (phosphodiesterase) inhibitors, such as theophylline, pentoxifylline, zaprinast, sildenafil, amrinone, milrinone, cilostamide, rolipram, and cilomilast; (27) phosphodiesterase-3B (PDE3B) inhibitors; (28) NE (norepinephrine) transport inhibitors, such as GW 320659, despiramine, talsupram, and nomifensine; (29) ghrelin receptor antagonists, such as those disclosed in PCT Application Nos. WO 01/87335, and WO 02/08250; (30) leptin, including recombinant human leptin (PEG-OB, Hoffman La Roche) and recombinant methionyl human leptin (Amgen); (31) leptin derivatives, such as those disclosed in U.S. Pat. Nos. 5,552,524, 5,552,523, 5,552,522, 5,521,283, and PCT International Publication Nos. WO 96/23513, WO 96/23514, WO 96/23515, WO 96/23516, WO 96/23517, WO 96/23518, WO 96/23519, and WO 96/23520; (32) BRS3 (bombesin receptor subtype 3) agonists such as [D-Phe6,beta-Ala11,Phe13,Nle14]Bn(6-14) and [D-Phe6,Phe13]Bn(6-13)propylamide, and those compounds disclosed in Pept. Sci. 2002 August; 8(8): 461-75); (33) CNTF (Ciliary neurotrophic factors), such as GI-181771 (Glaxo-SmithKIine), SR146131 (Sanofi Synthelabo), butabindide, PD170,292, and PD 149164 (Pfizer); (34) CNTF derivatives, such as axokine (Regeneron), and those disclosed in PCT Application Nos. WO 94/09134, WO 98/22128, and WO 99/43813; (35) monoamine reuptake inhibitors, such as sibutramine, and those disclosed in U.S. Pat. Nos. 4,746,680, 4,806,570, and 5,436,272, U.S. Patent Publication No. 2002/0006964 and PCT Application Nos. WO 01/27068, and WO 01/62341; (36) UCP-1 (uncoupling protein-1), 2, or 3 activators, such as phytanic acid, 4-[(E)-2-(5,6,7,8-tetrahydro-5,5,8,8-tetramethyl-2-naphthalenyl)-1-propenyl]benzoic acid (TTNPB), retinoic acid, and those disclosed in PCT Patent Application No. WO 99/00123; (37) thyroid hormone β agonists, such as KB-2611 (KaroBioBMS), and those disclosed in PCT Application No. WO 02/15845, and Japanese Patent Application No. JP 2000256190; (38) FAS (fatty acid synthase) inhibitors, such as Cerulenin and C75; (39) DGAT1 (diacylglycerol acyltransferase 1) inhibitors; (40) DGAT2 (diacylglycerol acyltransferase 2) inhibitors; (41) ACC2 (acetyl-CoA carboxylase-2) inhibitors; (42) glucocorticoid antagonists; (43) acyl-estrogens, such as oleoyl-estrone, disclosed in del Mar-Grasa, M. et al., Obesity Research, 9:202-9 (2001); (44) dipeptidyl peptidase IV (DP-IV) inhibitors, such as sitagliptin (Januvia™), NVP-DPP-728, vildagliptin (LAF 237), P93/01, denagliptin (GSK 823093), SYR322, RO 0730699, TA-6666, saxagliptin (BMS 477118), isoleucine thiazolidide, valine pyrrolidide, NVP-DPP728, LAF237, P93/01, TSL 225, TMC-2A/2B/2C, FE 999011, P9310/K364, VIP 0177, SDZ 274-444; and the compounds disclosed in U.S. Pat. No. 6,699,871, which is incorporated herein by reference; and International Patent Application Nos. WO 03/004498; WO 03/004496; EP 1 258 476; WO 02/083128; WO 02/062764; WO 03/000250; WO 03/002530; WO 03/002531; WO 03/002553; WO 03/002593; WO 03/000180; and WO 03/000181; (46) dicarboxylate transporter inhibitors; (47) glucose transporter inhibitors; (48) phosphate transporter inhibitors; (49) Metformin (Glucophage®); and (50) Topiramate (Topimax®); and (50) peptide YY, PYY 3-36, peptide YY analogs, derivatives, and fragments such as BIM-43073D, BIM-43004C (Olitvak, D. A. et al., Dig. Dis. Sci. 44(3):643-48 (1999)), and those disclosed in U.S. Pat. No. 5,026,685, U.S. Pat. No. 5,604,203, U.S. Pat. No. 5,574,010, U.S. Pat. No. 5,696,093, U.S. Pat. No. 5,936,092, U.S. Pat. No. 6,046,162, U.S. Pat. No. 6,046,167, U.S. Pat. No. 6,093,692, U.S. Pat. No. 6,225,445, U.S. Pat. No. 5,604,203, U.S. Pat. No. 4,002,531, U.S. Pat. No. 4,179,337, U.S. Pat. No. 5,122,614, U.S. Pat. No. 5,349,052, U.S. Pat. No. 5,552,520, U.S. Pat. No. 6,127,355, WO 95/06058, WO 98/32466, WO 03/026591, WO 03/057235, WO 03/027637, and WO 2004/066966, which are incorporated herein by reference; (51) Neuropeptide Y2 (NPY2) receptor agonists such NPY3-36, N acetyl [Leu(28,31)] NPY 24-36, TASP-V, and cyclo-(28/32)-Ac-[Lys28-Glu32]-(25-36)-pNPY; (52) Neuropeptide Y4 (NPY4) agonists such as pancreatic peptide (PP) as described in Batterham et al., J. Clin. Endocrinol. Metab. 88:3989-3992 (2003), and other Y4 agonists such as 1229U91; (54) cyclo-oxygenase-2 inhibitors such as etoricoxib, celecoxib, valdecoxib, parecoxib, lumiracoxib, BMS347070, tiracoxib or JTE522, ABT963, CS502 and GW406381, and pharmaceutically acceptable salts thereof; (55) Neuropeptide Y1 (NPY1) antagonists such as BIBP3226, J-115814, BIBO 3304, LY-357897, CP-671906, GI-264879A and those disclosed in U.S. Pat. No. 6,001,836; and PCT Application Nos. WO 96/14307, WO 01/23387, WO 99/51600, WO 01/85690, WO 01/85098, WO 01/85173, and WO 01/89528; (56) Opioid antagonists such as nalmefene (Revex®), 3-methoxynaltrexone, naloxone, naltrexone, and those disclosed in: PCT Application No. WO 00/21509; (57) 11β HSD-1 (11-beta hydroxy steroid dehydrogenase type 1) inhibitor such as BVT 3498, BVT 2733, and those disclosed in WO 01/90091, WO 01/90090, WO 01/90092, and U.S. Pat. No. 6,730,690 and US Publication No. US 2004-0133011, which are incorporated by reference herein in their entirety; and (58) aminorex; (59) amphechloral; (60) amphetamine; (61) benzphetamine; (62) chlorphentermine; (63) clobenzorex; (64) cloforex; (65) clominorex; (66) clortermine; (67) cyclexedrine; (68) dextroamphetamine; (69) diphemethoxidine, (70) N-ethylamphetamine; (71) fenbutrazate; (72) fenisorex; (73) fenproporex; (74) fludorex; (75) fluminorex; (76) furfurylmethylamphetamine; (77) levamfetamine; (78) levophacetoperane; (79) mefenorex; (80) metamfepramone; (81) methamphetamine; (82) norpseudoephedrine; (83) pentorex; (84) phendimetrazine; (85) phenmetrazine; (86) picilorex; (87) phytopharm 57; (88) zonisamide; (89) Neurokinin-1 receptor antagonists (NK-1 antagonists) such as the compounds disclosed in: U.S. Pat. Nos. 5,162,339, 5,232,929, 5,242,930, 5,373,003, 5,387,595, 5,459,270, 5,494,926, 5,496,833, and 5,637,699; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, and 97/49710; and 90) Qnexa.

Examples of other anti-obesity agents that can be employed in combination with a compound of formula I, II, III and IV are disclosed in “Patent focus on new anti-obesity agents,” Exp. Opin. Ther. Patents, 10: 819-831 (2000); “Novel anti-obesity drugs,” Exp. Opin. Invest. Drugs 9: 1317-1326 (2000); and “Recent advances in feeding suppressing agents: potential therapeutic strategy for the treatment of obesity, Exp. Opin. Ther. Patents, 11: 1677-1692 (2001). The role of neuropeptide Y in obesity is discussed in Exp. Opin. Invest. Drugs, 9: 1327-1346 (2000). Cannabinoid receptor ligands are discussed in Exp. Opin. Invest. Drugs, 9: 1553-1571 (2000).

The instant invention also includes administration of a single pharmaceutical dosage formulation which contains both a ghrelin antagonist/inverse agonist in combination with a second active ingredient, as well as administration of each active agent in its own separate pharmaceutical dosage formulation. Where separate dosage formulations are used, the individual components of the composition can be administered at essentially the same time, i.e., concurrently, or at separately staggered times, i.e. sequentially prior to or subsequent to the administration of the other component of the composition. The instant invention is therefore to be understood to include all such regimes of simultaneous or alternating treatment, and the terms “administration” and “administering” are to be interpreted accordingly. Administration in these various ways are suitable for the present compositions as long as the beneficial pharmaceutical effect of the combination of the ghrelin antagonist/inverse agonist and the second active ingredient is realized by the patient at substantially the same time. Such beneficial effect is preferably achieved when the target blood level concentrations of each active ingredient are maintained at substantially the same time. It is preferred that the combination of the ghrelin antagonist/inverse agonist and the second active ingredient be co-administered concurrently on a once-a-day dosing schedule; however, varying dosing schedules, such as the ghrelin antagonist/inverse agonist once a day and the second active ingredient once, twice or more times per day or the ghrelin antagonist/inverse agonist three times a day and the second active ingredient once, twice or more times per day, is also encompassed herein. A single oral dosage formulation comprised of both a ghrelin antagonist/inverse agonist and a second active ingredient is preferred. A single dosage formulation will provide convenience for the patient, which is an important consideration especially for patients with diabetes or obese patients who may be in need of multiple medications.

The compounds in the combinations of the present invention may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of the ghrelin receptor antagonist/inverse agonist, or a pharmaceutically acceptable salt or ester thereof, and a pharmaceutically acceptable carrier or diluent in a first unit dosage form, and a second unit dosage form comprising a prophylactically or therapeutically effective amount of the second active ingredient or drug, or a pharmaceutically acceptable salt or ester thereof, and a pharmaceutically acceptable carrier or diluent in a second unit dosage form. In one embodiment, the kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

Another aspect of the present invention provides pharmaceutical compositions which comprise a compound of formula I, II, III or IV, as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of formula I, II, III and IV can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the typical oral dosage unit form, in which case solid pharmaceutical carriers are typically employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I, II, III and IV may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fingi. The carrier can be a solvent or dispersion medium containing, for example, water, ethanol, polyol (e.g. glycerol, propylene glycol and liquid polyethylene glycol), suitable mixtures thereof, and vegetable oils.

The compounds of structural formula I, II, III and IV of the present invention can be prepared according to the procedures of the following Schemes and Examples, using appropriate materials and are further exemplified by the following specific examples. The compounds illustrated in the examples are not, however, to be construed as forming the only genus that is considered as the invention. The Examples further illustrate details for the preparation of the compounds of the present invention. Those skilled in the art will readily understand that known variations of the conditions and processes of the following preparative procedures can be used to prepare these compounds. The instant compounds are generally isolated in the form of their pharmaceutically acceptable salts, such as those described previously hereinabove. The free amine bases corresponding to the isolated salts can be generated by neutralization with a suitable base, such as aqueous sodium hydrogencarbonate, sodium carbonate, sodium hydroxide, and potassium hydroxide, and extraction of the liberated amine free base into an organic solvent followed by evaporation. The amine free base isolated in this manner can be further converted into another pharmaceutically acceptable salt by dissolution in an organic solvent followed by addition of the appropriate acid and subsequent evaporation, precipitation, or crystallization. All temperatures are degrees Celsius unless otherwise noted. Mass spectra (MS) were measured by electron-spray ion-mass spectroscopy.

The phrase “standard peptide coupling reaction conditions” means coupling a carboxylic acid with an amine using an acid activating agent such as EDC, DCC, and BOP in an inert solvent such as dichloromethane in the presence of a catalyst such as HOBT. The use of protecting groups for the amine and carboxylic acid functionalities to facilitate the desired reaction and minimize undesired reactions is well documented. Conditions required to remove protecting groups are found in standard textbooks such as Greene, T, and Wuts, P. G. M., Protective Groups in Organic Synthesis, John Wiley & Sons, Inc., New York, N.Y., 1991. CBZ and BOC are commonly used protecting groups in organic synthesis, and their removal conditions are known to those skilled in the art. For example, CBZ may be removed by catalytic hydrogenation in the presence of a noble metal or its oxide such as palladium on activated carbon in a protic solvent such as methanol or ethanol. In cases where catalytic hydrogenation is contraindicated due to the presence of other potentially reactive functionalities, removal of CBZ groups can also be achieved by treatment with a solution of hydrogen bromide in acetic acid or by treatment with a mixture of TFA and dimethylsulfide. Removal of BOC protecting groups is carried out with a strong acid, such as trifluoroacetic acid, hydrochloric acid, or hydrogen chloride gas, in a solvent such as methylene chloride, methanol, or ethyl acetate.

Abbreviations Used in the Description of the Preparation of the Compounds of the Present Invention: BOC (Boc) is t-butyloxycarbonyl, BOP is benzotriazol-1-yloxytris(dimethylamino)-phosphonium hexafluorophosphate, Bn is benzyl, Bu is butyl, calc. or calc'd is Calculated, celite is Celite™ diatomaceous earth, CBZ (Cbz) is benzyloxycarbonyl, c-hex is cyclohexyl, c-pen is cyclopentyl, c-pro is cyclopropyl, DCM is dichloromethane, DEAD is diethyl azodicarboxylate, DIEA is diisopropyl-ethylamine, DMAP is 4-dimethylaminopyridine, DMF is N,N-dimethylformamide, dppf is 1,1′-Bis(diphenylphosphino)ferrocene, EDC is 1-(3-dimethylaminopropyl)3-ethylcarbodiimide HCl, eq is equivalent(s), ES-MS is electron spray ion-mass spectroscopy, Et is ethyl, EtOAc is ethyl acetate, h or hr is hour(s), HATU is O-(7-azabenzotriazol-1-yl)-1,1,3,3-tetramethyluronium hexafluorophosphate, HOAt is 1-hydroxy-7-azabenzotriazole, HOBt is 1-hydroxybenzotriazole hydrate, HPLC is high performance liquid chromatography, LC-MS or LC-MASS is liquid chromatography mass spectrum, LDA is lithium diisopropylamide, Me is methyl, MF is molecular formula, MS is mass spectrum, Ms is methane sulfonyl, NBS is N-bromosuccinamide, NMM is N-Methylmorpholine, NMO is N-Methylmorpholine-N-oxide, OTf is trifluoromethanesulfonyl, Ph is phenyl, Phe is phenyl alanine, Pr is propyl, iPr is isopropyl, prep. is prepared, PyBrop is bromo-tris-pyrrolidino-phosphonium hexafluorophosphate, r.t. or rt is room temperature, Tf is triflate or trifluoromethanesulfonate, TFA is trifluoroacetic acid, THF is tetrahydrofuran, and TLC is thin-layer chromatography.

Reaction Schemes 1-9 illustrate methods employed in the synthesis of the compounds of the present invention of structural formula I, II, III and IV. All substituents are as defined above unless indicated otherwise.

Several methods for preparing the compounds of this invention are illustrated in the following Schemes and Examples. Starting materials are either commercially available or made by known procedures in the literature or as illustrated.

One general way of constructing target compounds of formula I wherein X=NH, and R₂=H) is shown in Scheme 1. Phenyl acyl bromide 1A can react with dimethyl sulfide in solvents such as benzene to give intermediate 1B, which can react with diazonium salts 1C in solvents such as water and ethanol at 0° C. or elevated temperature to give intermediate 1D. Intermediate 1D may be reacted with an R1 group, such as a substituted nitrile, to yield the targeted compounds of formula I.

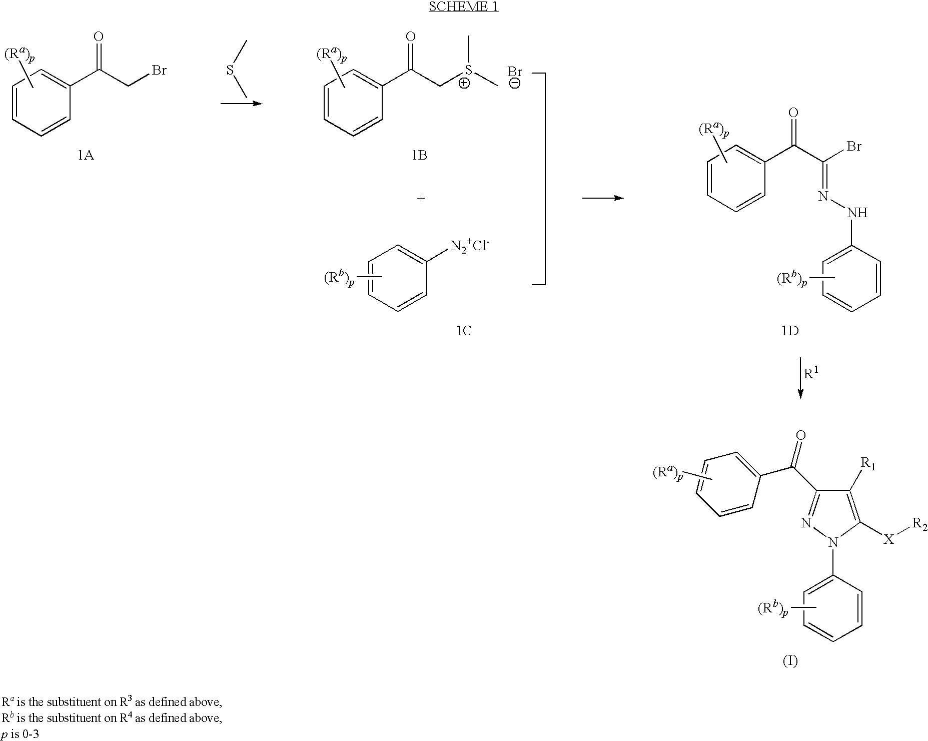

Another way of constructing target compounds of formula I wherein X=NH, R₁=CN, and R₂=H) is shown in Scheme 2. Phenyl acyl bromide 1A may be reacted with malononitrile in the presence of bases such as NaOH in solvents such as water and ethanol at room temperature to yield intermediate 2A, which can react with diazonium salts 2B in solvents such as water and ethanol at 0° C. or elevated temperature to yield compound 2C.

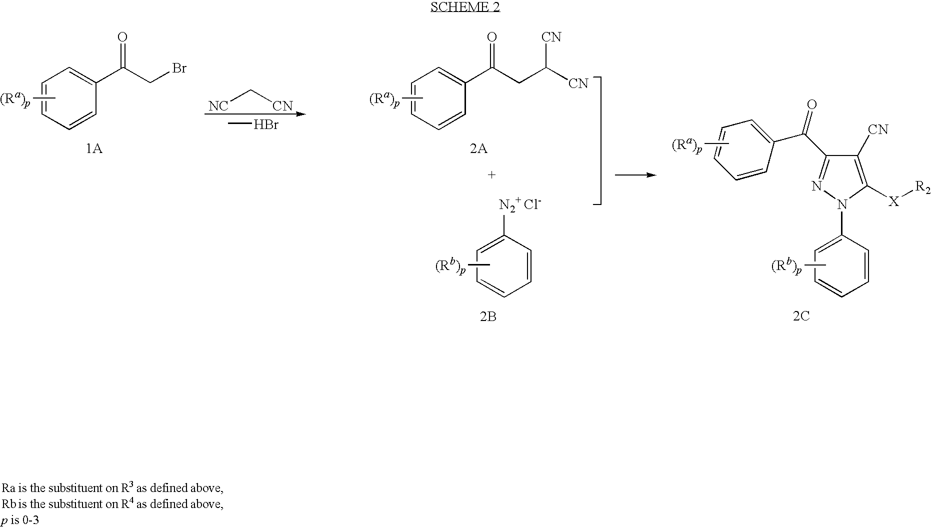

In cases where phenyl acyl bromide 1A is not available, targeted compounds I can be synthesized as shown in Scheme 3. Intermediate 3A-1 (prepared according to Tetrahedron Vol 39, 1983, 129-136) may be reacted with malononitrile to yield ester 3B. The ester 3B is then hydrolyzed in the presence of base such as LiOH in protic solvents such as water and methanol to yield acid 3C after acidification with strong acids such as concentrated hydrochloric acid. The acid 3C may be coupled to N,O-dimethyl hydroxylamine in the presence of coupling reagents such EDC or DCC in solvents such as methylene chloride to yield key intermediate 3D. Intermediate 3D may then be reacted with various organo-lithium reagents and Grignard reagents at temperature range from −78° C. to 80° C. in solvents such as THF to give compound 3E, which can be converted to compounds of formula I.

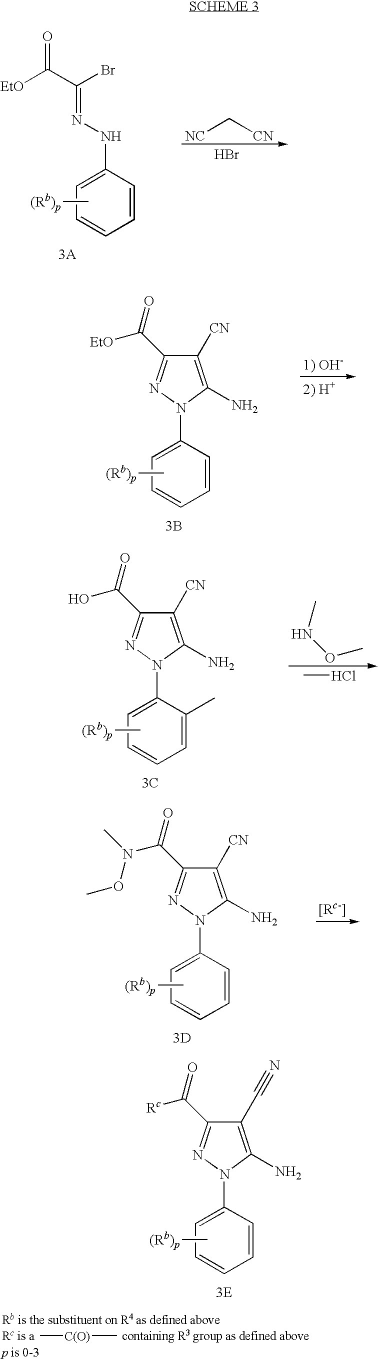

In cases where the substituents on the pyrazole nitrogen are not substituted phenyls, compound of formula I can be synthesized according to Scheme 4. Diethyloxalate 4A may be reacted with substituted nitrile in the presence of bases such as NaOEt and NaO^{t}Bu in solvents such as ethanol at room temperature or elevated temperature to yield intermediate 4B. Intermediate 4B is then treated with POCl₃ at 110° C. followed by methanol quenching at 0° C. to yield intermediate 4C. Intermediate 4C may be reacted with substituted hydrazines 4D in the presence of bases such as triethylamine in alcoholic solvents such as ethanol to yield aminopyrazole 4E. Intermediate 4E may be further reacted with various organo-lithium reagents and Grignard reagents at temperature range from −78° C. to 80° C. in solvents such as THF to give compound 4F, which can be converted to compounds of formula I.

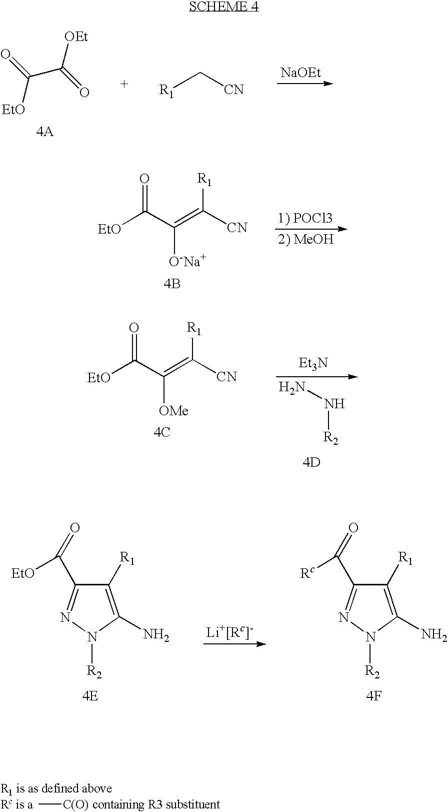

When it is desired to prepare compounds of formula I wherein R₂ is not a hydrogen, the compounds of general formula I in which R₂=H may be further modified using the methodology described below in reaction Scheme 5. For example, compound 5A (I) (R₂=H) may be directly alkylated using an alkylating agent such as SB in a polar aprotic solvent such as DMF. In this reaction, the substituent leaving group, LG, of compound 5B is a leaving group such as a halide, mesylate or triflate, and the product is the compound of formula I bearing the R₂ substituent.

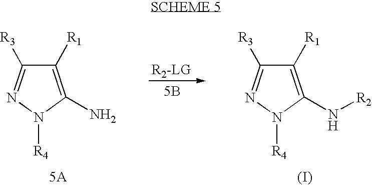

When it is desired to prepare compounds of structural formula I wherein X is NH(CH₂)₃ and R₂ is NH₂, the compounds 6A, of formula I wherein X=NH and R₂=H, may be further modified using the methodology described below in Scheme 6. For example, compound 6A may be directly alkylated using an alkylating agent such as 6B in a polar aprotic solvent such as DMF in the presence of a base such as K₂CO₃ or NaH. The N-Phth protected compound 6C may be deprotected under conditions for instance by treatment with methylamine in refluxing methanol or using hydrazine in ethanol to yield compounds 6D.

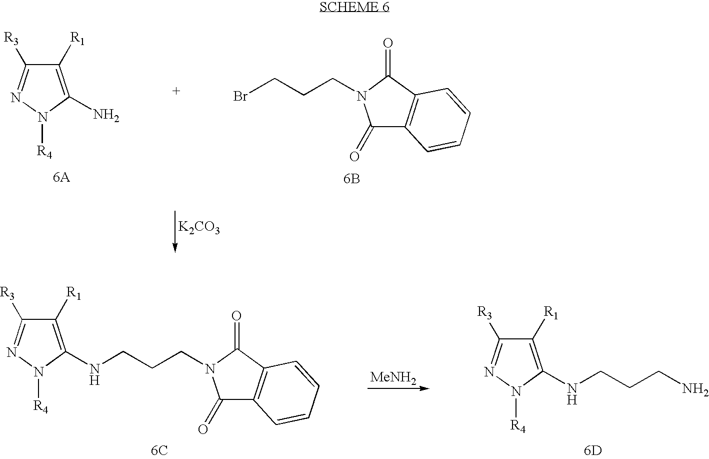

When it is desired to prepare compounds of structural formula I wherein X is CH₂-pyrrolidine and R₂ is NH₂, the targeted compounds may be synthesized using the methodology described below in reaction Scheme 7. For example, compound 7A may be condensed with 4-Chloro-3-oxo-butyric acid ethyl ester in the presence of a base such as NaOEt in a protic solvent such as EtOH to give ethyl ester compounds 7B. Treatment of 7B with MeAl(Cl)NH₂ followed by refluxing in POCl₃ yield nitrile 7C. Compound 7C may be directly alkylated using alkylating agents, such as 7D in a polar aprotic solvent such as DMF in the presence of a base such as K₂CO₃ or NaH to give compounds 7E. Further removal of BOC protecting group may be achieved using strong acids such as TFA in solvents such as CH₂Cl₂ to 2 give compound 7F

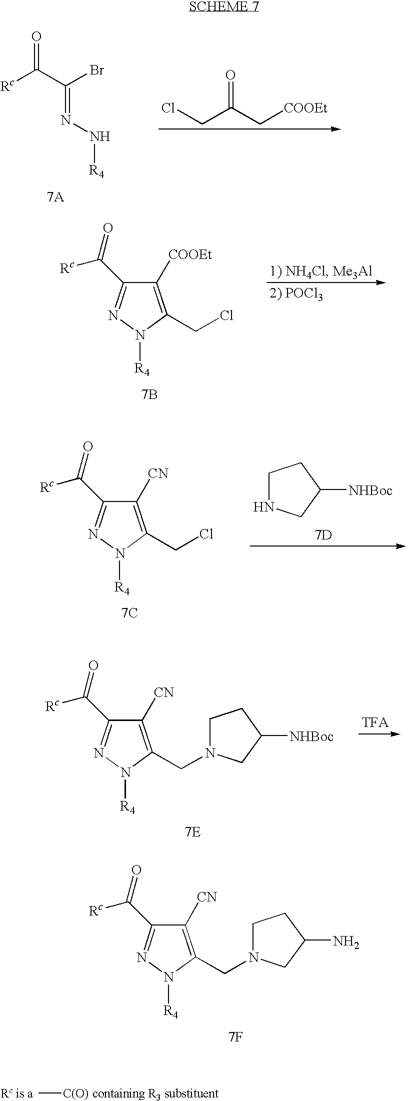

The resulting compounds of structural formula I wherein R₂=NH₂, such as 6D and 7F, may then be subjected to one of several alkylation/acylation strategies known in organic chemistry to add another R group (Scheme 8). For example, compounds of formula I in which X=NH(CH₂)₃ and R₂=NH₂ may be utilized in a reductive amination reaction with a suitable carbonyl containing reagent. The reductive amination is achieved by initial formation of an imine between the amine of formula 8A and either an aldehyde or ketone of formula 8B. The intermediate imine is then treated with a reducing agent, such as sodium cyanoborohydride or sodium triacetoxyborohydride, to give the alkylated product 8D. Alternatively, compound 8A may be directly alkylated using an alkylating agent such as 8C in a polar aprotic solvent such as DMF to give compound 8D, a compound of formula I in which X=NH(CH₂)₃, and R₂=NCHRₐR_{b}. In this reaction, the substituent leaving group, LG, of compound 8C is a leaving group such as a halide, mesylate or triflate. Compound 8A may be coupled to carboxylic acids, such as 8E, and acid chlorides, such as 8G, in the presence of coupling reagents, such as DCC or EDC, in aprotic solvent, such as THF and CH₂Cl₂, to give amide 8F and carbamate 8H. Compound 8A may also be reacted with reagents such as 8I, and 8K) in aprotic solvent such as THF and CH₂Cl₂, to give carbamide 8J and sulfonamide 8L.

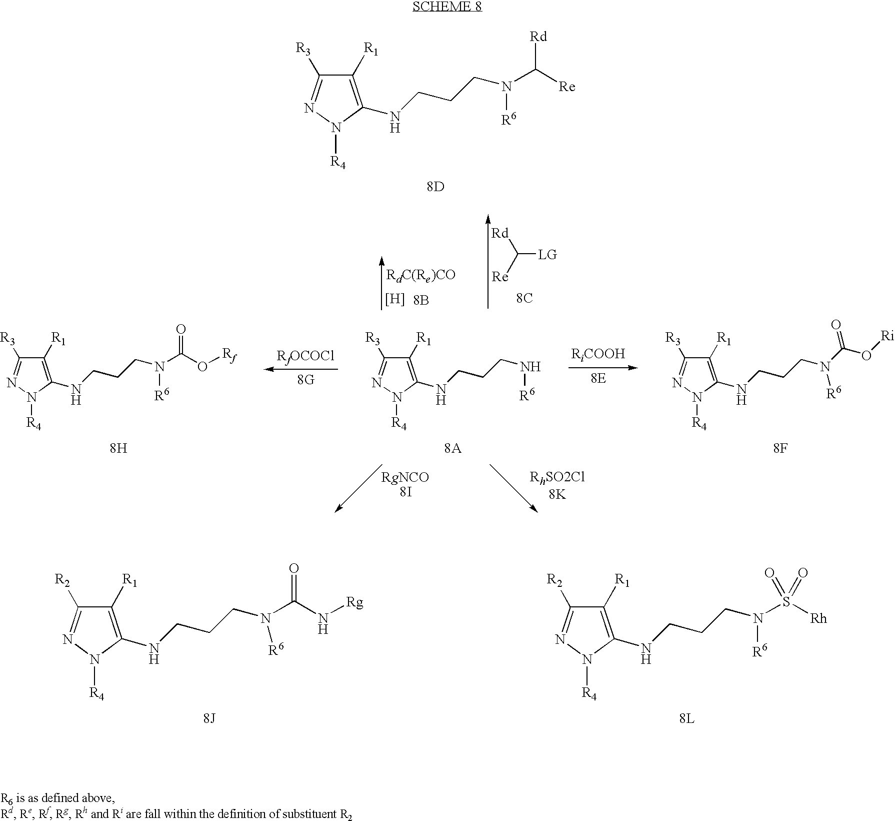

When it is desired to prepare compounds of structural formula I wherein X is NH(CH₂)₃ and R₂ is N(R_{b})C(O)Rₐ, the targeted compounds may be synthesized using the methodology described below in reaction Scheme 9. For example, compound 9A may be treated with (BOC)₂O in a base, such as pyridine, to give compound 9B, which can be further alkylated with alkylating reagents such as 9C in the presence of a base such as K₂CO₃ or NaH in aprotic solvents, such as THF or DMF, to give compound 9D. Compound 9D may be reacted with reagents such as 9E and 9H in the presence of a base such as K₂CO₃, NaH or KHMDS to give compounds 9F and 9I. Further removal of BOC protecting group may be achieved using a strong acid such as TFA or HCl in a solvent such as CH₂Cl₂ or dioxane to give compounds 9G and 9J.

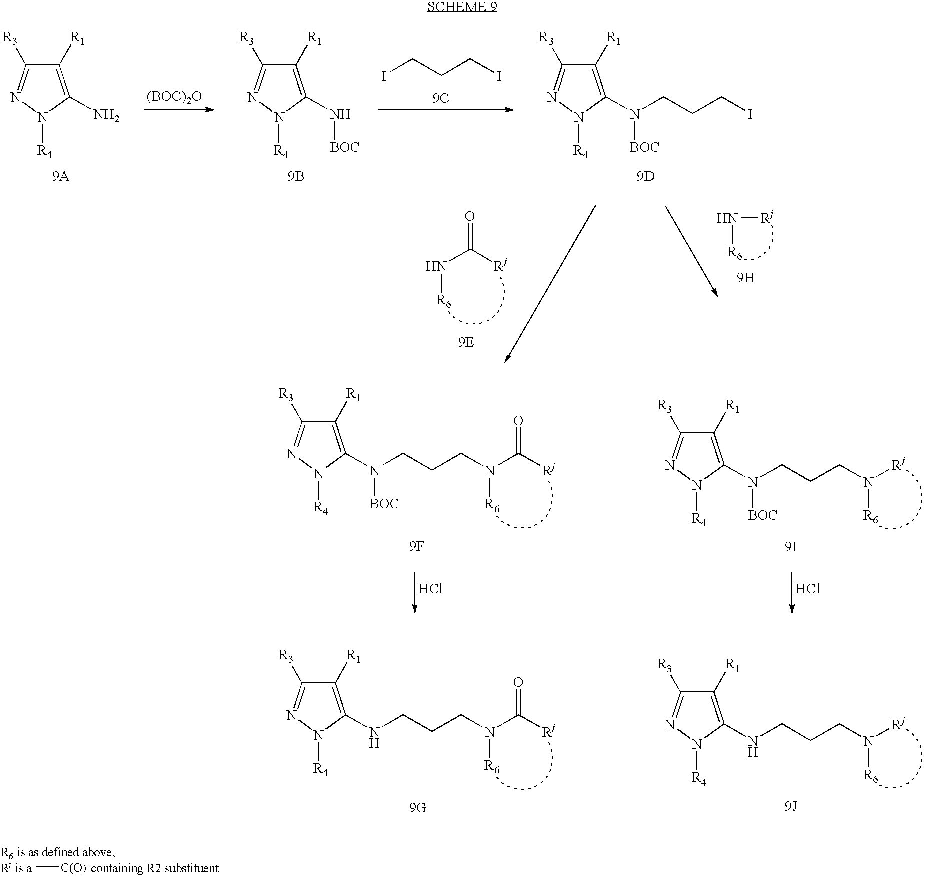

The following Intermediates and Examples are provided to illustrate the invention and are not to be construed as limiting the invention in any manner. The scope of the invention is defined by the appended claims.

### Intermediate 1

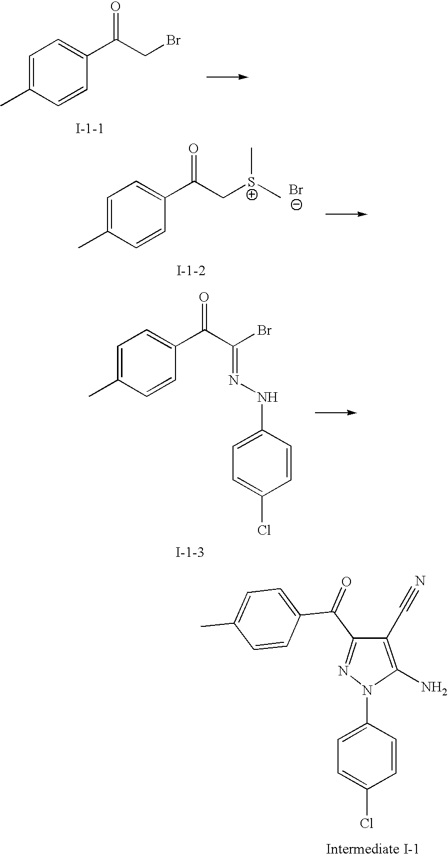

Step A: To compound I-1-1 (21.307 g, 100.0 mmol) was added benzene (50 mL), and the reaction was stirred until dissolution. To the reaction solution was added dimethyl sulfide (7.33 mL, 100 mmol). The reaction was vigorously stirred for 24 h under ambient conditions. The resulting solid was isolated by vacuum filtration and washed with cold benzene and ethanol to yield compound I-1-2 as a white solid.
Step B: To a cooled solution of 4-chloroaniline (2.551 g, 20.0 mmol) in 6 N aqueous HCl (12 mL) was slowly added a cooled solution of sodium nitrite (1.38 g, 20 mmol) in water (20 mL), keeping the reaction below 5° C. at all times. The resulting diazonium salt solution was then stirred at 0° C. for 30 minutes. To a cooled solution of compound I-1-2 (5.51 g, 20.0 mmol) in ethanol (50 mL) was added a solution of sodium acetate (8.00 g) in water (30 mL), and the resulting ylide solution was cooled to 0° C. With vigorous stirring, to the sulfonium ylide solution were added small portions of the diazonium salt solution, keeping the temperature below 5° C. at all times. The reaction was then stirred at 0° C. for 4 h and then stored in the refrigerator overnight. The resulting solid was isolated by vacuum filtration and washed with cold ethanol to yield compound I-1-3.
Step C: To compound I-1-3 (150 mg, 0.427 mmol) and malonitrile (50 mg, 0.757 mmol) was added absolute ethanol (6 mL). To the solution was added a 21 weight percent solution of sodium ethoxide in ethanol (0.34 mL, 1.2 mmol), and the reaction was stirred under ambient conditions for 1.5 h. The resulting solid was isolated by vacuum filtration and washed with cold ethanol to yield Intermediate I-1. ¹H NMR (500 MHz, d6-DMSO) δ 8.06 (d, 2H), 7.63 (s, 4H), 7.33 (d, 2H), 7.02 (s, 2H), 2.37 (s, 3H).

The compounds in Table 1 were prepared using the appropriate reagents following procedures similar to that described above for Intermediate I-1.

TABLE 1

| | | | Mass |
|---|---|---|---|
| | | | Found |
| Intermediate | Structure | Formula | [M + H] |
| I-2 | 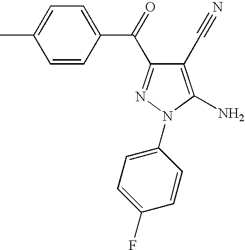 | C18H13FN4O | 321.1 |
| I-3 | 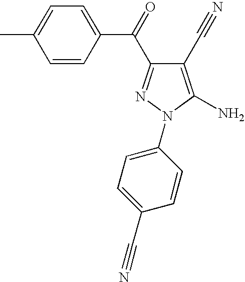 | C19H13N5O | 328.1 |
| I-4 | 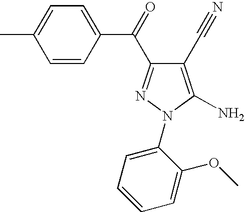 | C19H16N4O2 | 333.1 |
| I-5 | 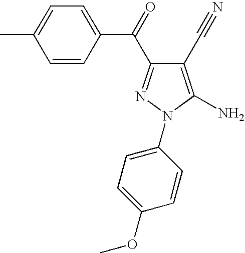 | C19H16N4O2 | 333.2 |
| I-6 | 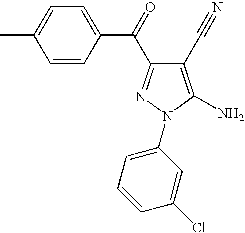 | C18H13ClN4O | 337.1 |
| I-7 | 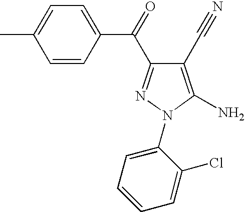 | C18H13ClN4O | 337.2 |
| I-8 | 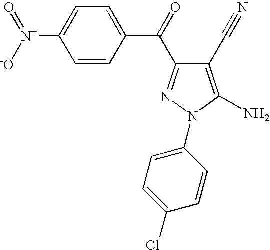 | C17H10ClN5O3 | 368.1 |
| I-9 | 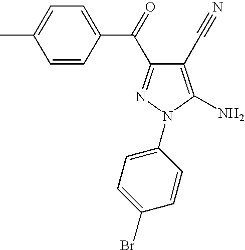 | C18H13BrN4O | 380.9 |
| I-10 | 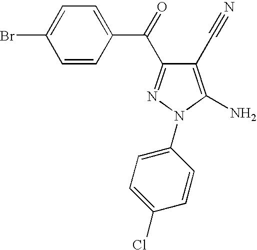 | C17H10BrClN4O | 401.0 |

### Intermediate I-11

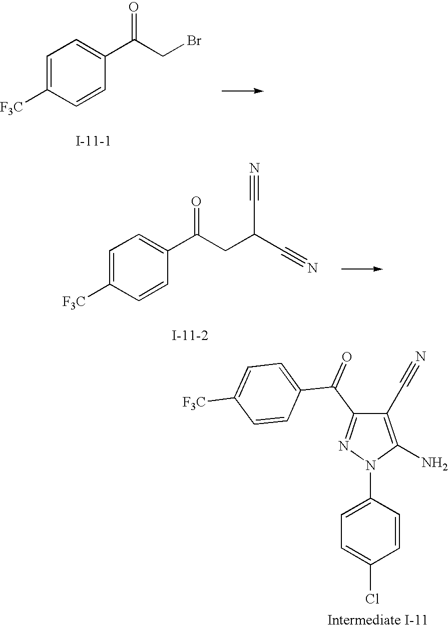

Step A: To compound I-11-1 (48.1 g, 180.1 mmol) and malonitrile (29.7 mg, 450.0 mmol) was added ethanol (180 mL), and the reaction was stirred until dissolution. To the reaction solution was added an aqueous solution of NaOH (1 N, 180.1 mL). The reaction was vigorously stirred for 30 min under ambient conditions. To the completed reaction was added ethanol (180 mL), and the suspension was stirred for 5 min. The resulting solid was isolated by vacuum filtration and washed with a cold 50% EtOH/H₂O solution to yield compound I-11-2 as a white solid.
Step B. To a cooled solution of 4-chloroaniline (13.4 g, 105 mmol) in 6 N aqueous HCl (63 mL) was slowly added a cooled solution of sodium nitrite (7.25 g, 105 mmol) in water (110 mL), keeping the reaction below 5° C. at all times. The diazonium salt solution was then stirred at 0° C. for 30 min. To a cooled solution of compound I-11-2 (25.2 g, 100 mmol) in pyridine (500 mL) was slowly added the diazonium salt solution, keeping below 5° C. at all times. The reaction was allowed to warm to ambient temperature overnight and then diluted with water (1 L). The resulting solid was isolated by vacuum filtration and washed with a cold ethanol to yield Intermediate 11 as a yellow solid. ¹H NMR (500 MHz, d6-DMSO) δ 8.29 (d, 2H), 7.89 (d, 2H), 7.63 (s, 4H), 7.11 (s, 2H).

The compounds in Table 2 were prepared using the appropriate reagents following procedures similar to that described above for Intermediate 1-11.

TABLE 2

| | | | Mass |
|---|---|---|---|
| | | | Found |
| Intermediate | Structure | Formula | [M + H] |
| I-12 | 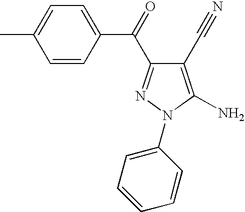 | C18H14N4O | 303.1 |
| I-13 | 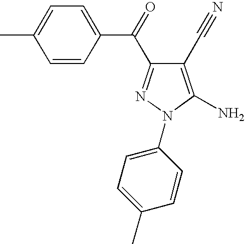 | C19H16N4O | 317.2 |
| I-14 | 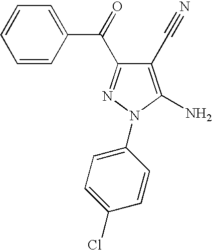 | C17H11ClN4O | 323.1 |
| I-15 | 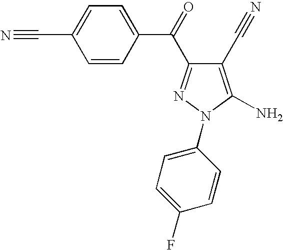 | C18H10FN5O 331.312 | 332.5 |
| I-16 | 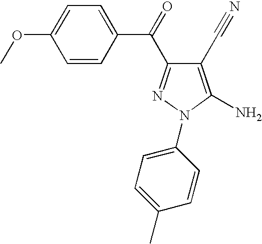 | C19H16N4O2 | 333.2 |
| I-17 | 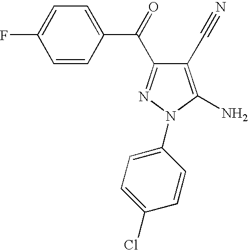 | C17H10ClFN4O | 341.2 |
| I-18 | 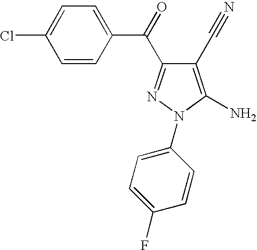 | C17H10ClFN4O | 341.0 |
| I-19 | 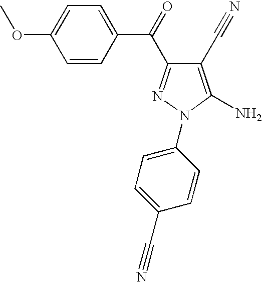 | C19H13N5O2 | 343.9 |
| I-20 | 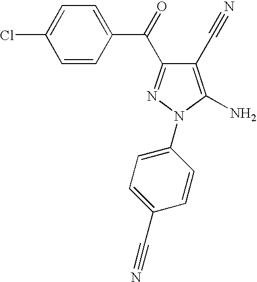 | C18H10ClN5O | 347.9 |
| I-21 | 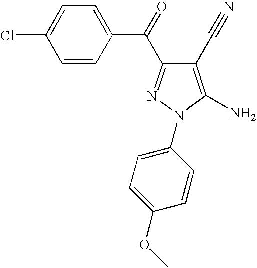 | C18H13ClN4O2 | 353.2 |
| I-22 | 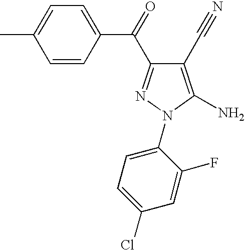 | C18H12ClFN4O | 354.9 |
| I-23 | 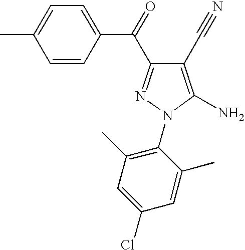 | C20H17ClN4O | 365.3 |
| I-24 | 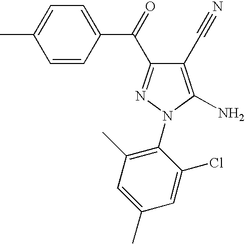 | C20H17ClN4O | 365.3 |
| I-25 | 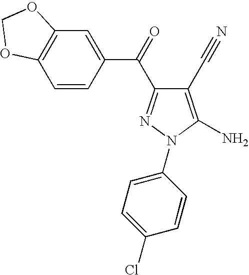 | C18H11ClN4O3 | 367.2 |
| I-26 | 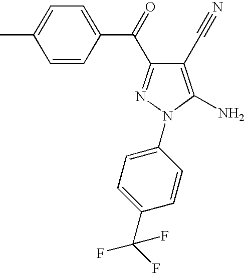 | C19H13F3N4O | 371.1 |
| I-27 | 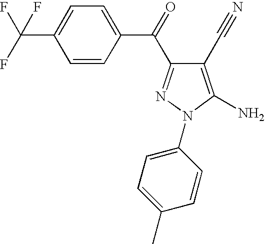 | C19H13F3N4O | 371.2 |
| I-28 | 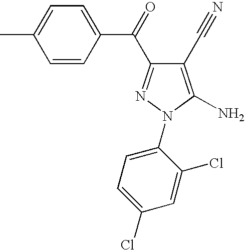 | C18H12Cl2N4O | 371.0 |
| I-29 | 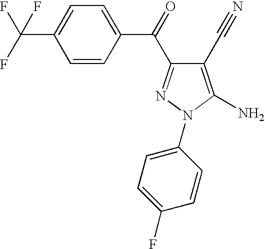 | C18H10F4N4O | 374.9 |
| I-30 | 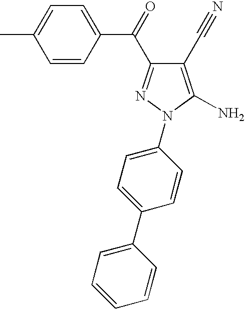 | C24H18N4O | 379.3 |
| I-31 | 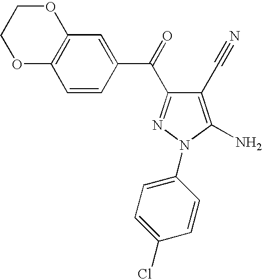 | C19H13ClN4O3 | 381.2 |
| I-32 | 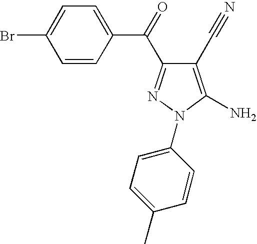 | C18H13BrN4O | 381.0 |
| I-33 | 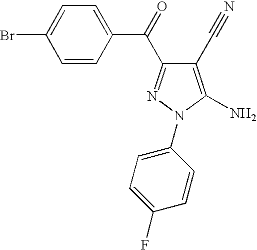 | C17H10BrFN4O | 385.2 |
| I-34 | 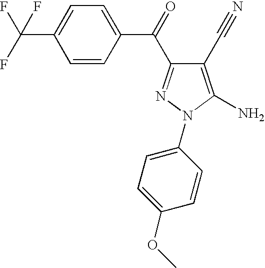 | C19H13F3N4O2 | 387.2 |
| I-35 | 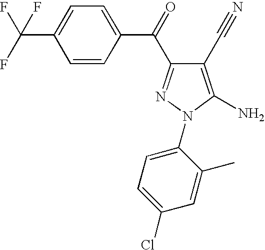 | C19H12ClF3N4O | 405.0 |
| I-36 | 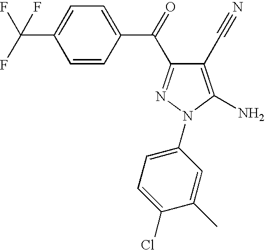 | C19H12ClF3N4O | 405.3 |
| I-37 | 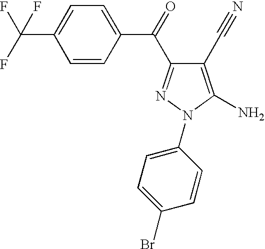 | C18H10BrF3N4O | 434.8 |
| I-38 | 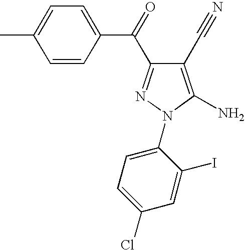 | C18H12ClIN4O | 463.0 |
| I-39 | 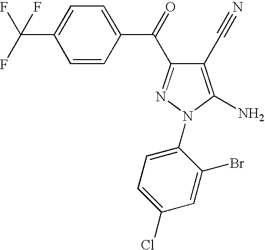 | C18H9BrClF3N4O | 469.0 |
| I-40 | 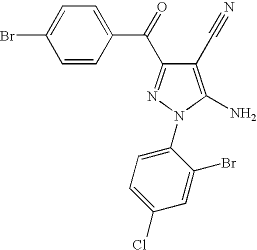 | C17H9Br2ClN4O | 480.7 |
| I-41 | 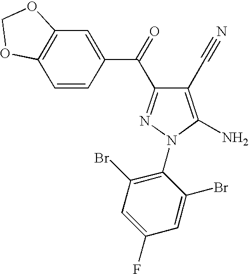 | C18H9Br2FN4O3 | 508.9 |

### Intermediate 42

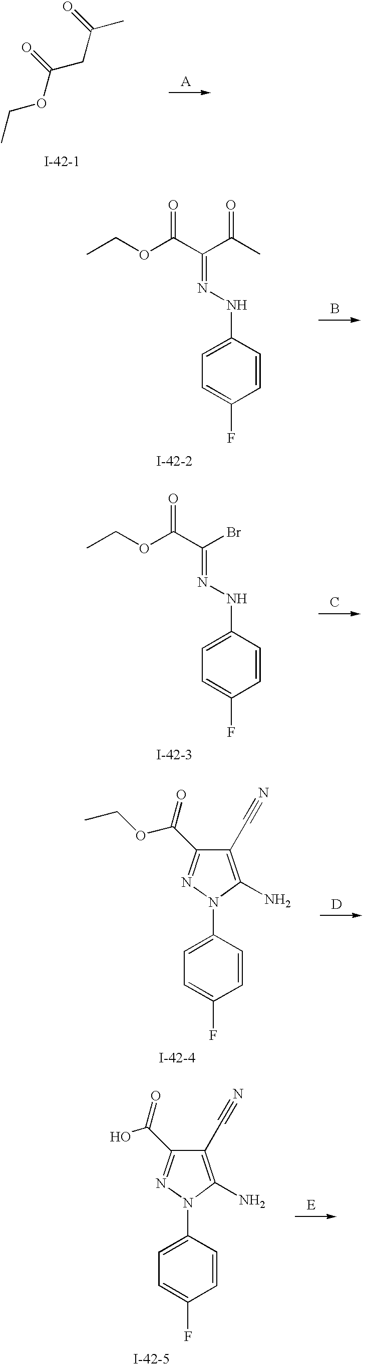

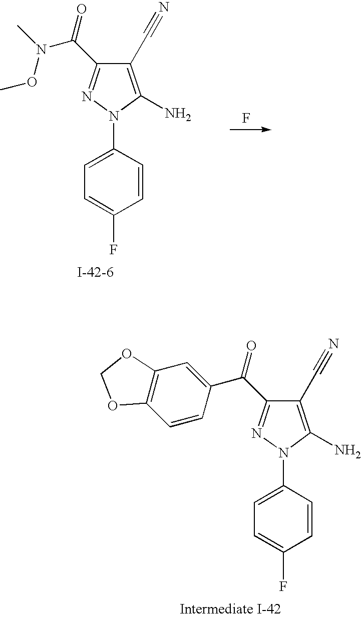

Step A: To a cooled solution of 4-fluoroaniline (41.7 g, 375 mmol) in 6 N aqueous HCl (225 mL) was added a solution of sodium nitrite (25.9 g, 375 mmol) in water (125 mL), while keeping the reaction below 5° C. at all times with addition of excess ice to the reaction. The resulting diazonium salt solution was then stirred at 0° C. for 30 min. To a solution of Compound I-42-1 (48.8 g, 375 mmol) in ethanol (280 mL) was added a solution of NaOAc (94 g) in water (500 mL) and excess ice, followed by addition of the diazonium salt solution, keeping the reaction temperature below 5° C. at all times. The reaction was stirred for 2 h at 0° C. The resulting solid was isolated by vacuum filtration and washed with water to yield compound I-42-2.
Step B: To a solution of NaOAc (56.2 g) in acetic acid (623 mL) and acetic anhydride (206 mL) was added compound I-42-2 (70.9 g, 281 mmol) and cooled to 0° C. To the cooled reaction was slowly added a solution of bromine (14.5 mL, 281 mmol) in acetic acid (187 mL) over 1 hour, followed by stirring for 30 min at 0° C. The completed reaction was poured into ice water (2 L) and then stirred overnight under ambient conditions. The resulting solid was isolated by vacuum filtration. This crude product recrystallized from ethanol/water, isolated by vacuum filtration, and washed with cold ethanol to yield compound I-42-3.
Step C: To compound I-42-3 (61.4 g, 212 mmol) and malonitrile (14.0 g, 212 mmol) was added ethanol (900 mL), and the reaction was stirred until dissolution. To the reaction solution was added a 21 weight percent solution of sodium ethoxide in ethanol (198 mL, 530 mmol), and the reaction was stirred under ambient conditions for 2 h. The reaction was concentrated in vacuo to yield crude compound I-42-4.
Step D: To crude compound I-42-4 (212 mmol) added methanol (500 mL) and tetrahydrofuran (500 mL). To this solution was added a solution of lithium hydroxide monohydrate (89 g, 212 mmol) in water (250 mL). The reaction was stirred overnight under ambient conditions. The reaction was concentrated in vacuo, diluted with water, washed with ether, acidified, and extracted with ethyl acetate. The organic extracts were dried and concentrated in vacuo to yield compound I-42-5.
Step E: To compound I-42-5 (5.0 g, 20 mmol) was added N,O-dimethylhydroxylamine hydrochloride (4.13 g, 50 mmol), diisopropylethylamine (12 mL, 65 mmol), and dichloromethane (120 mL). To the reaction solution was added DMAP (490 mg, 4 mmol) and EDAC-HCl coupling reagent (9.59 g, 50 mmol). The reaction was stirred under nitrogen at ambient temperature overnight. The reaction was concentrated in vacuo and purified by column chromatography to yield compound I-42-6.
Step F: To compound I-42-6 (1.45 g, 5.0 mmol) was added THF (25 mL), and the solution was cooled to −78° C. To the cold solution was added 3,4-(methylenedioxy)-phenyl magnesium bromide solution (1 M in THF/toluene, 11 mL). The reaction was stirred under nitrogen at −78° C. for 2 hours. After an aqueous workup, the reaction was purified by column chromatography to yield Intermediate I-42. LC-MS for C₁₈H₁₁FN₄O₃ [M+H]: calculated 351.1, found 351.3.

Following essentially the procedures outlined for Intermediate 1-42, the Intermediates I-43 to I-59 listed in Table 3 were prepared.

TABLE 3

| | | | Mass Found |
|---|---|---|---|
| Intermediate | Structure | Formula | [M + H] |
| I-43 | 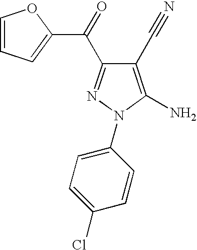 | C15H9ClN4O2 | 313.5 |
| I-44 | 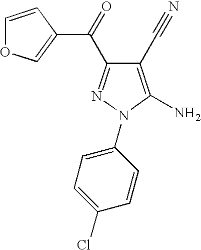 | C15H9ClN4O2 | 313.3 |
| I-45 | 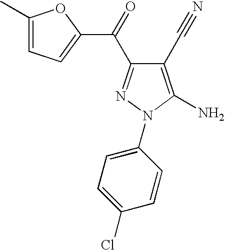 | C16H11ClN4O2 | 327.7 |
| I-46 | 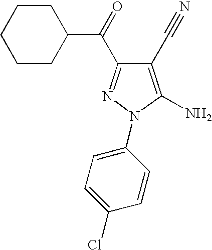 | C17H17ClN4O | 329.2 |
| I-47 | 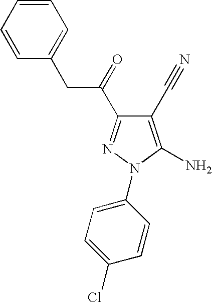 | C18H13ClN4O | 337.1 |
| I-48 | 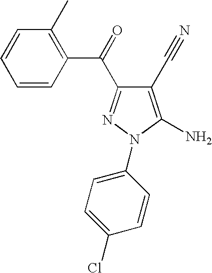 | C18H13ClN4O | 337.1 |
| I-49 | 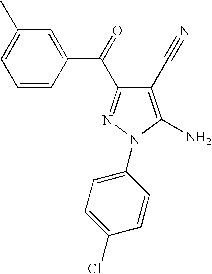 | C18H13ClN4O | 337.1 |
| I-50 | 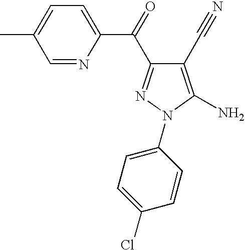 | C17H12ClN5O | 338.1 |
| I-51 | 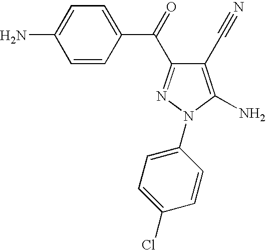 | C17H12ClN5O | 338.4 |
| I-52 | 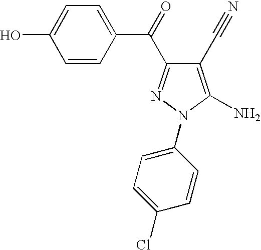 | C17H11ClN4O2 | 339.2 |
| I-53 | 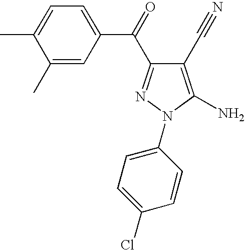 | C19H15ClN4O | 351.1 |
| I-54 | 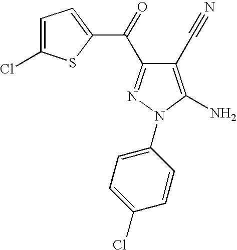 | C15H8Cl2N4OS | 363.1 |
| I-55 | 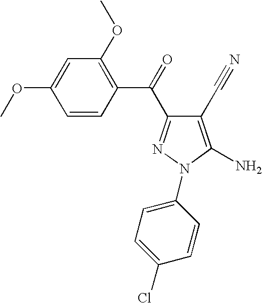 | C19H15ClN4O3 | 383.1 |
| I-56 | 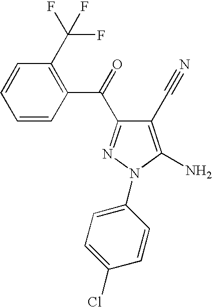 | C18H10ClF3N4O | 391.0 |
| I-57 | 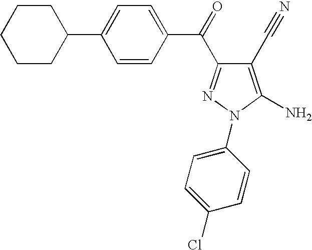 | C23H21ClN4O | 405.2 |
| I-58 | 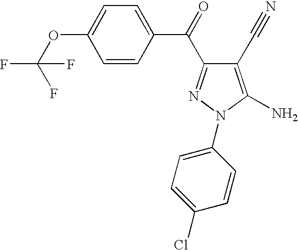 | C18H10ClF3N4O2 | 406.8 |
| I-59 | 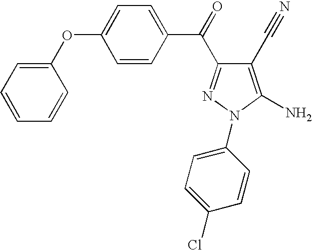 | C23H15ClN4O2 | 415.1 |
| I-60 | 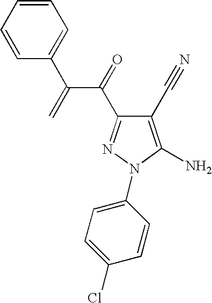 | C19H13ClN4O | 349.0 |

### Intermediate 61

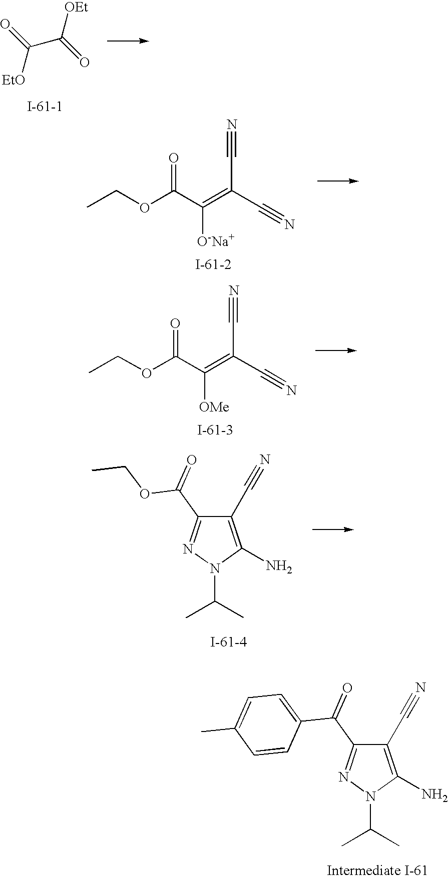

Step A: To malonitrile (39.94 g, 600 mmol) was added ethanol (900 mL) and compound I-61-1 (86.3 mL, 600 mmol). To the reaction was added dropwise a 21 weight percent solution of NaOEt in ethanol (224 mL, 600 mmol). The reaction was stirred for 1 h under ambient conditions, and then concentrated in vacuo. To the crude product was added ethyl acetate (1 L), and the solution was stirred for 1 h under ambient conditions. The solid was removed by vacuum filtration, and the filtrate was concentrated in vacuo. The filtrate product was then triturated in EtOH/Ether, isolated by vacuum filtration, and lyophilized from water to yield compound I-61-2 as a white solid.
Step B: To freshly azeotroped compound I-61-2 (15.0 g, 79.7 mmol) in a dry flask was added POCl₃ (40 mL, 430 mmol). The reaction was stirred under nitrogen at ambient temperature until dissolution, then heated to 110° C. for 30 min, and allowed to cool to ambient temperature. The reaction was concentrated in vacuo to remove all POCl₃. With cooling in an ice water bath, to the crude intermediate was slowly added methanol (200 mL). Upon completion of exotherm, the reaction was concentrated in vacuo and purified by flash chromatography (40% EtOAc/Hexanes) to yield the compound I-61-3 as a yellow liquid.
Step C: To a suspension of i-propylhydrazine-HCl (765 mg, 6.9 mmol) in ethanol (10 mL) was added triethylamine (1.1 mL, 7.6 mmol), and the suspension was stirred under nitrogen until dissolution. To the solution was added a solution of compound I-61-3 (1.5 g, 8.3 mmol) in ethanol (1.5 mL). The reaction was stirred 15 minutes under nitrogen at ambient temperature. The completed reaction was concentrated in vacuo and purified by flash chromatography (gradient 0-20% EtOAc/DCM) to give compound I-61-4 as a yellow solid.
Step D: To a solution of compound I-61-4 (147 mg, 0.662 mmol) in THF (3 mL) was added 4-methylphenylmagnesium bromide (1.4 mL of 1 M solution in ether, 1.4 mmol) at −30° C. The reaction was stirred for 1 hour, and then quenched with saturated potassium sodium tartrate aqueous solution (15 mL). The aqueous layer was extracted with EtOAc (2×15 mL). The combined organic layers were concentrated and purified by reverse phase HPLC(YMC-Pack Pro C18, 100×20 mm, 5 μm, gradient 20-100 CH₃CN/H₂O with 0.1% TFA) to yield Intermediate I-61. ¹H NMR (500 MHz, d6-DMSO) δ 8.04 (d, 2H), 7.33 (d, 2H), 6.87 (broad, 2H), 4.57 (m, 1H), 2.38 (s, 3H), 1.33 (d, 6H).

The compounds in Table 4 were prepared using the appropriate reagents following procedures similar to that described above for Intermediate I-61.

TABLE 4

| | | | Mass Found |
|---|---|---|---|
| Intermediate | Structure | Formula | [M + H] |
| I-62 | 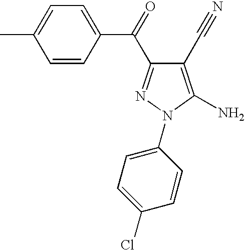 | C17H14ClN3O | 312.0 |
| I-63 | 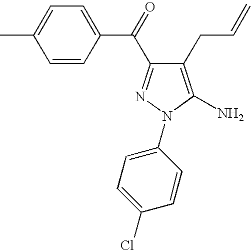 | C20H18ClN3O | 352.6 |
| I-64 | 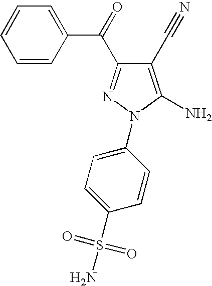 | C17H13N5O3S | 368.1 |
| I-65 | 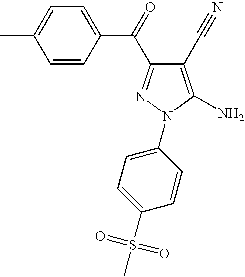 | C19H16N4O3S | 381.2 |
| I-66 | 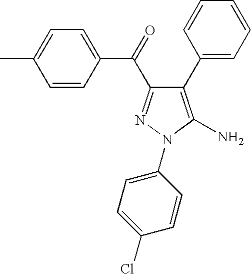 | C23H18ClN3O | 388.2 |

### Intermediate I-67

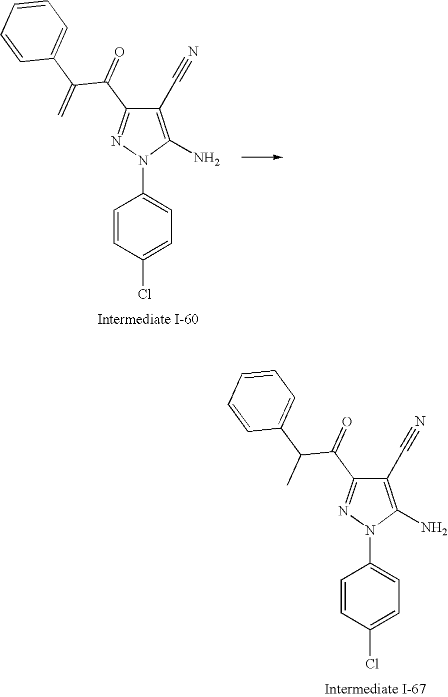

Step A: To Intermediate I-60 (4.33 mg) was added EtOH (1 mL) and 10% palladium on carbon (2.3 mg). The reaction was stirred under hydrogen (1 atm) at ambient temperature for 3 h. The catalyst was removed by vacuum filtration through celite, and the filtrate was concentrated in vacuo to yield Intermediate I-67. LCMS for C₁₉H₁₅ClN₄O [M+H]: expected 351.1, found 351.1.

### Intermediate I-68

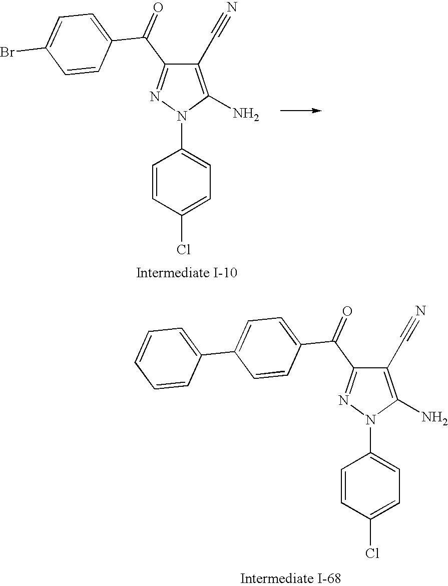

Step A: To Intermediate I-10 (16 mg, 0.04 mmol) was added phenyl boronic acid (7 mg, 0.06 mmol), K₂CO₃ (21 mg, 0.15 mmol), Combiphos POPd (4.5 mg, 0.005 mmol, Combiphos Catalysts, Inc.), and THF (1 mL). The reaction was refluxed under nitrogen overnight. The completed reaction was purified by reverse-phase HPLC (YMC-Pack Pro C18, 100×20 mm, 5 μm, gradient 20-100 CH₃CN/H₂O with 0.1% TFA) to yield Intermediate I-68. LCMS for C₂₃H₁₅ClN₄O [M+H]: expected 399.1, found 399.1.

### Intermediate 69

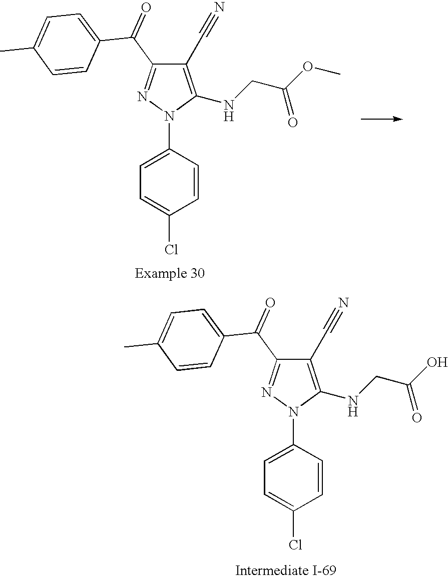

Step A: To a solution of Example 30 (18 mg, 0.044 mmol) in MeOH/THF/H₂O (4:4:1, 1 mL) was added LiOH.H₂O (18 mg, 0.44 mmol). The reaction was stirred under ambient conditions for 18 h. The completed reaction was purified by reverse phase HPLC (YMC-Pack Pro C18, 100×20 mm, 5 μm, gradient 10-80 CH₃CN/H₂O with 0.1% TFA) to yield Intermediate I-69. LCMS for C₂₉H₃₁ClN₄O₅ [M+H⁺]: calculated 395.1, found 395.3.

The compounds in Table 5 were prepared using the appropriate reagents following procedures similar to that described above for Intermediate I-69.

TABLE 5

| | | | Mass |
|---|---|---|---|
| | | | Found |
| Intermediate | Structure | Parent Formula | [M + H] |
| 70 | 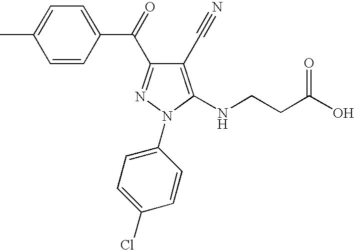 | C21H17ClN4O3 | 409.1 |
| 71 | 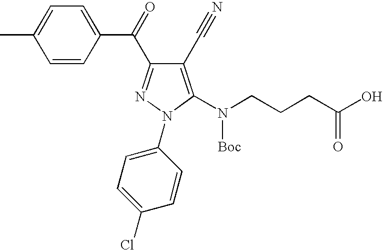 | C27H27ClN4O5 | 523.3 |

### Intermediate 72

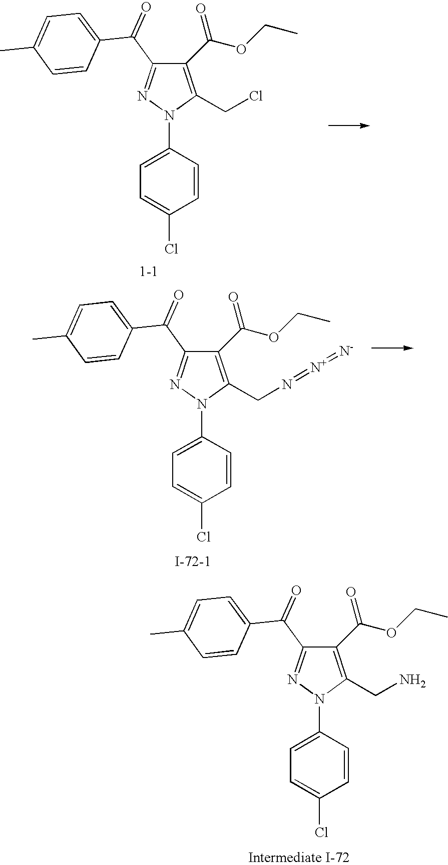

Step A.—To compound 1-1 (30 mg, 0.072 mmol) was added sodium azide (20 mg, 0.308 mmol) and DMF (1 mL). The reaction was stirred under ambient conditions for 2 h. The crude product was isolated by aqueous workup and purified by prep TLC to yield compound I-72-1. LC-MS for C₂₁H₁₈ClN₅O₃ [M+H]: calculated 424.1, found 424.1.
Step B.—To a solution of compound I-72-1 (20 mg) in EtOH (2 mL) was added 10% Pd/C (20 mg). The reaction was stirred under hydrogen (1 atm) at ambient temperature for 1.5 h. The completed reaction was purified by reverse-phase HPLC (YMC-Pack Pro C18, 100×20 mm, 5 μm, gradient 20-100 CH₃CN/H₂O with 0.1% TFA) to yield Intermediate I-72. LC-MS for C₂₁H₂₀ClN₃O₃ [M+H]: calculated 398.1, found 398.2.

### Intermediate 73

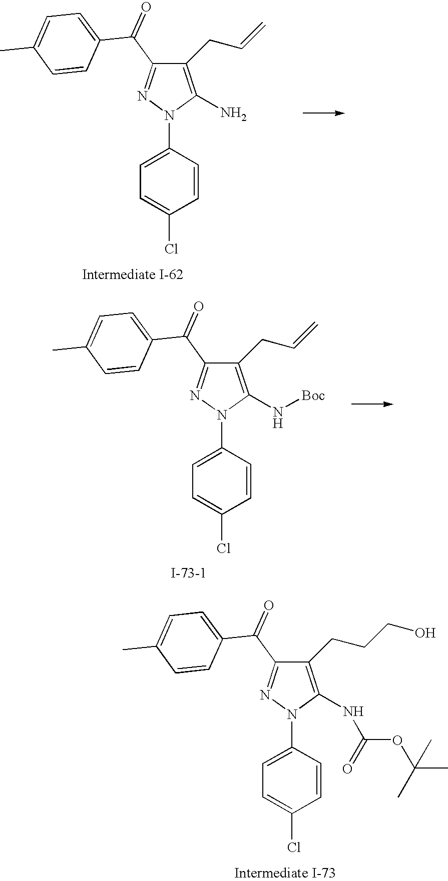

Step A: To Intermediate I-62 (3.52 g, 10.0 mmol) was added pyridine (50 mL) and di-tert-butyl carbonate (6.54 g, 30.0 mmol), and the reaction was stirred under ambient conditions for 18 h. The completed reaction was concentrated in vacuo. To the crude di-Boc intermediate was added THF/MeOH/H₂O (2:2:1, 170 mL) and LiOH.H₂O (4.06 g, 96.6 mmol), and the reaction was stirred under ambient conditions for 12 h. The product was isolated by aqueous workup to yield the intermediate I-73-1. LCMS for C₂₅H₂₆ClN₃O₃ [M+H⁺]: calculated 452.2, found 452.8.
Step B: To a solution of cyclohexene (720 μL, 7.08 mmol) in THF (10 mL) was added a solution of BH₃ (1 M in THF, 3.6 mL). The solution was stirred under nitrogen at ambient temperature for 1 h. To the solution was added a solution of intermediate I-73-1 (533 mg, 1.18 mmol) in THF (5 mL). The reaction was stirred under nitrogen at ambient temperature for 18 h. To the reaction was added NaBO₄.4H₂O (˜5 g). The reaction was stirred under nitrogen at ambient temperature for 48 h. The crude product was isolated by aqueous workup and purified by column chromatography to yield Intermediate I-73. LC-MS for C₂₅H₂₈ClN₃O₄ [M+H]: calculated 470.2, found 470.8.

### Intermediate 74

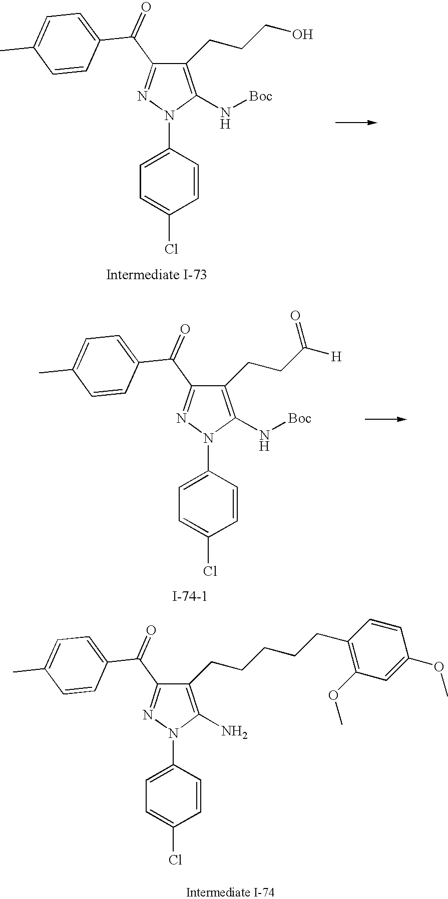

Step A: To a solution of Intermediate I-73 (140 mg, 0.30 mmol) in DCM (4 mL) was added a solution of Dess-Martin reagent (15% in DCM, 2 mL), and the reaction was stirred under ambient conditions for 1 h. To the completed reaction was added silica gel followed by vacuum filtration. The filtrate was purified by column chromatography to yield the intermediate I-74-1. LCMS for C₂₅H₂₆ClN₃O₄ [M+H⁺]: calculated 468.2, found 468.5.
Step B: To a solution of intermediate I-74-1 (127 mg, 0.272 mmol) and 2,4-dimethoxybenzyl amine (66.5 mg, 0.306 mmol) in THF (2 mL) was added triethylamine (53 μL, 0.326 mmol). The reaction was stirred under nitrogen at ambient temperature for 1 h. To the reaction was added NaBH(AcO)₃ (˜115 mg), and the reaction was stirred under nitrogen at ambient temperature for 2 h. The crude product was isolated by aqueous workup to yield the crude Boc-protected product. To the protected intermediate was added a solution of TFA (20% in DCM, 4 mL), and the reaction was stirred under ambient conditions for 4 h. The reaction was purified by column chromatography to yield Intermediate I-74. LC-MS for C₂₉H₃₁ClN₄O₃ [M+H]: calculated 519.2, found 519.9.

### Example 1

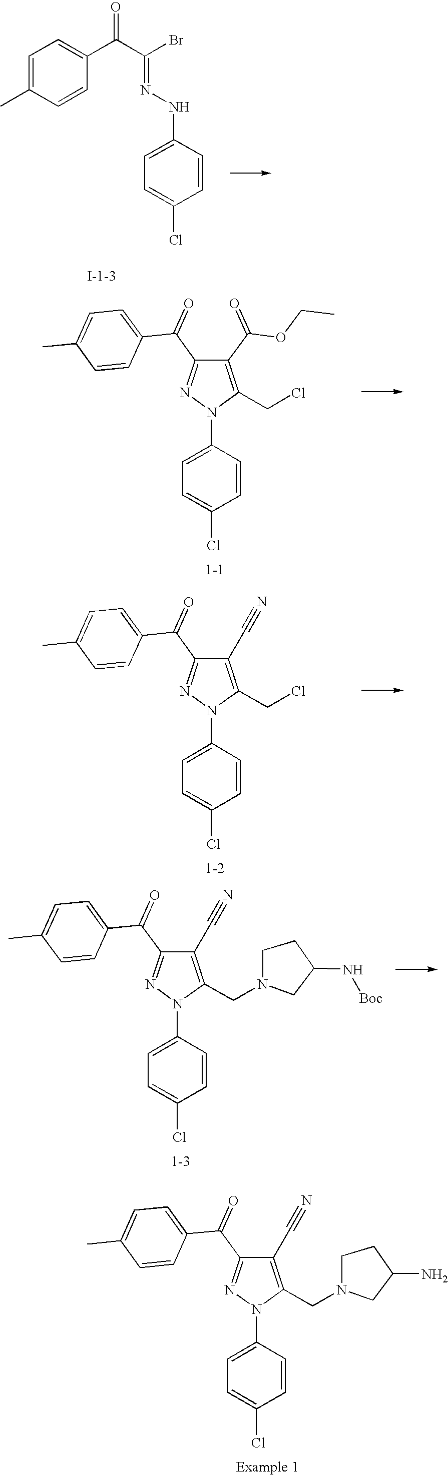

Step A.—To a solution of Intermediate I-1-3 (1.086 g, 3.09 mmol) in ethanol (10 mL) was added ethyl 4-chloroacetoacetate (417 μL, 3.09 mmol) and a 21 weight percent solution of sodium ethoxide in ethanol (2.3 mL). The reaction was stirred under ambient conditions for 1 h. The crude product was isolated by aqueous workup and purified by column chromatography to yield compound 1-1. LC-MS for C₂₁H₁₈Cl₂N₂O₃ [M+H]: calculated 417.1, found 417.7.
Step B.—To freshly azeotroped ammonium chloride (160 mg, 3 mmol) was added benzene (15 mL), and the solution was cooled to 0° C. under nitrogen. To the cooled solution was slowly added trimethylaluminum (2 M toluene, 1.5 mL, 3 mmol), and the solution was allowed to warm to ambient temperature for 2 h. To this solution was added a solution of compound 1-1 (400 mg, 0.96 mmol) in benzene (2 mL). The reaction was stirred under nitrogen at 80° C. for 4 h. The completed reaction was concentrated in vacuo. To the residue was added POCl₃ (6 mL), and the reaction was stirred under nitrogen at 110° C. for 3 h. The crude product was isolated by aqueous workup and purified by column chromatography to yield compound 1-2. LC-MS for C₁₉H₁₃Cl₂N₃O [M+H]: calc. 370.0, found 370.3.
Step C.—To compound 1-2 (30 mg, 0.082 mmol) was added 3-(Boc-amino) pyrrolidine (30 mg, 0.161 mmol), diisopropylethyl amine (10 μL), and DMF (0.5 mL). The reaction was stirred under nitrogen at ambient temperature for 18 h. The completed reaction was purified by reverse-phase HPLC (YMC-Pack Pro C18, 100×20 mm, 5 μm, gradient 20-100 CH₃CN/H₂O with 0.1% TFA) to yield compound 1-3. LC-MS for C₂₈H₃₀ClN₅O₃ [M+H]: calculated 520.2, found 520.6.
Step D.—To compound 1-3 (20 mg, 0.039 mmol) was added 4 N HCl in dioxane (5 mL). The reaction was stirred under ambient conditions for 4 h. The completed reaction was lyophilized directly to yield Example 1. LC-MS for C₂₃H₂₂ClN₅O [M+H]: calculated 420.2, found 420.4.

The compounds in Table 10 were prepared using the appropriate reagents following procedures similar to that described above for Example 1.

TABLE 10

| | | | Mass Found |
|---|---|---|---|
| Example | Structure | Formula | [M + H] |
| 2 | 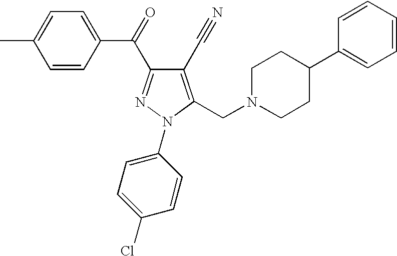 | C30H27ClN4O | 495.1 |
| 3 | 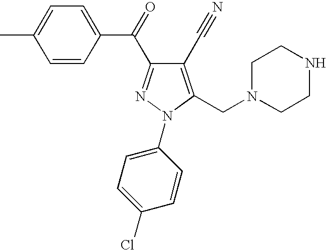 | C23H22ClN5O | 419.9 |
| 4 | 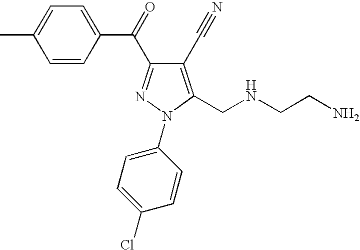 | C21H20ClN5O | 394.2 |
| 5 | 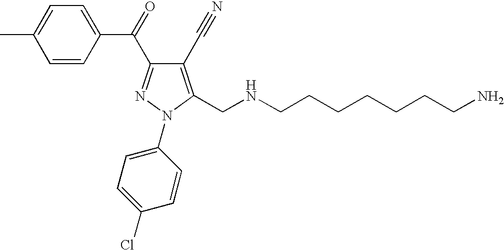 | C26H30ClN5O | 464.4 |
| 6 | 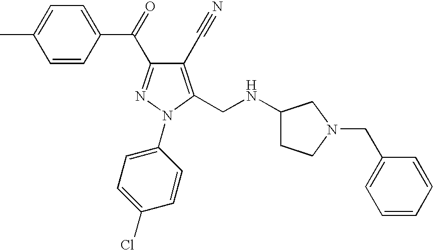 | C30H28ClN5O | 510.6 |

### Example 7

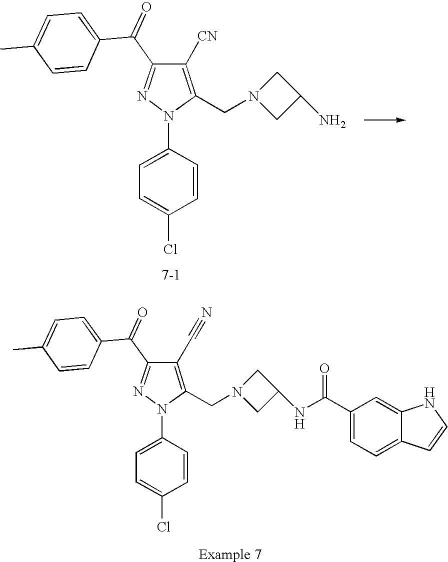

Step A: Intermediate 7-1 was prepared using 3-N-Boc-amino-azetidine following procedures similar to that described above for Example 1.
Step B: Example 7 was prepared using Intermediate 7-1 and indole-6-carboxylic acid following procedures similar to that described for Example 43. LC-MS calculated for C31H25ClN6O2: 548, Observed 549 [M+H].

### Example 8

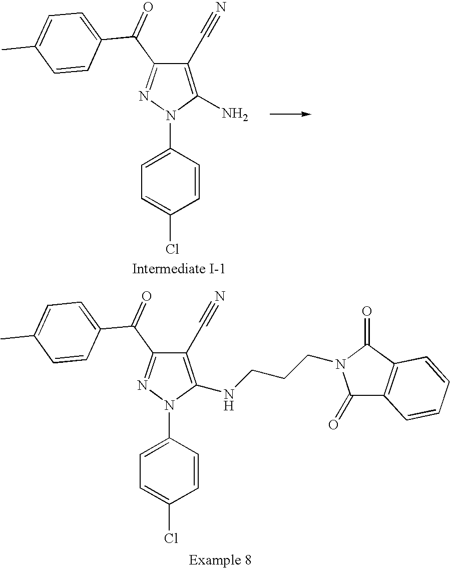

To Intermediate I-1 (9.38 g, 27.9 mmol) was added KOtBu (3.75 g, 33.4 mmol) and DMF (110 mL). The reaction was stirred under nitrogen for 30 min at ambient temperature. To the reaction was added N-(3-bromopropyl)phthalamide (11.21 g, 41.8 mmol) and NaI (418 mg, 2.79 mmol). The reaction was stirred under nitrogen at ambient temperature overnight. The crude product was isolated by aqueous workup and column chromatography to yield Example 8. ¹H NMR (500 MHz, CDCl₃) δ 8.21 (d, 2H), 7.86 (m, 2H), 7.77 (m, 2H), 7.60 (s, 4H), 7.29 (d, 2H), 5.33 (t, 1H), 3.80 (m, 2H) 3.63 (m, 2H), 2.44 (s, 3H), 2.08 (m, 2H).

The compounds in Table 11 were prepared using the appropriate reagents following procedures similar to that described above for Example 8.

TABLE 11

| | | | Mass Found |
|---|---|---|---|
| Example | Structure | Parent Formula | [M + H] |
| 9 | 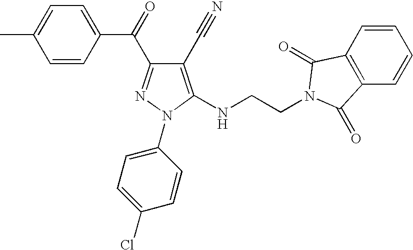 | C28H20ClN5O3 | 510.0 |
| 10 | 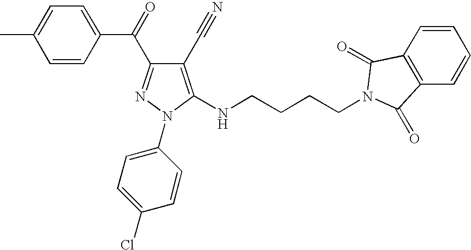 | C30H24ClN5O3 | 538.1 |

### Example 11

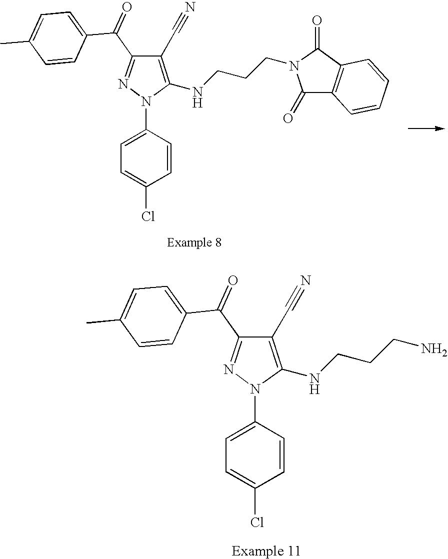

To Example 8 (˜14 mmol) in ethanol (250 mL) was added a 33% solution of methylamine in ethanol (35 mL, 140 mmol). The reaction was heated at 60° C. under nitrogen overnight to afford clean deprotection of the amine. The product was isolated by aqueous workup and then purified by column chromatography to yield Example 11. ¹H NMR (500 MHz, d6-DMSO) δ 8.04 (d, 2H), 7.83 (broad, 2H), 7.67 (m, 2H), 7.64 (m, 2H), 7.33 (d, 2H), 7.05 (broad, 1H), 3.48 (t, 2H), 2.84 (t, 2H), 2.37 (s, 3H), 1.89 (m, 2H).

The compounds in Table 12 were prepared using the appropriate reagents following procedures similar to that described above for Example 11.

TABLE 12

| | | | Mass |
|---|---|---|---|
| | | | Found |
| Example | Structure | Parent Formula | [M + H] |
| 12 | 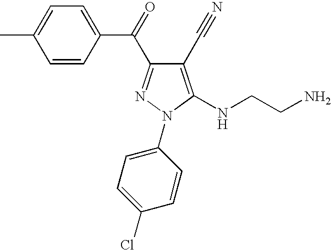 | C20H18ClN5O | 380.3 |
| 13 | 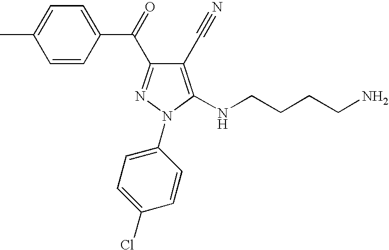 | C22H22ClN5O | 408.2 |
| 14 | 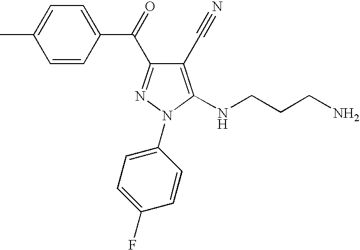 | C21H20FN5O | 378.5 |
| 15 | 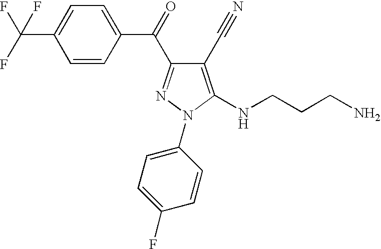 | C21H17F4N5O | 432.5 |
| 16 | 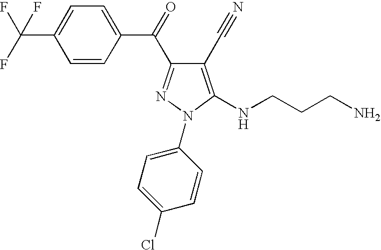 | C21H17ClF3N5O | 448.4 |
| 17 | 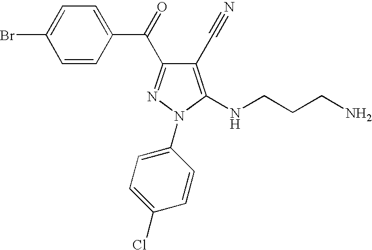 | C20H17BrClN5O | 458.6 |
| 18 | 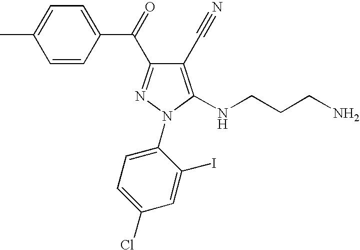 | C21H19ClIN5O | 520.4 |
| 19 | 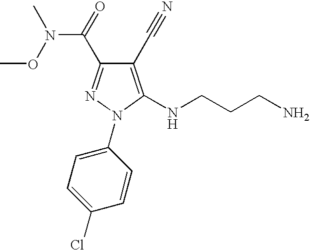 | C16H19ClN6O2 | 363.3 |
| 20 | 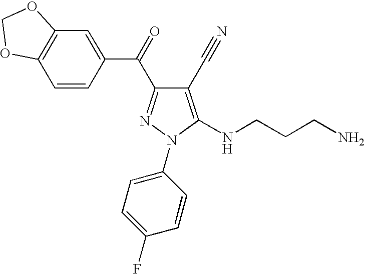 | C21H18FN5O3 | 408.3 |

### Example 21

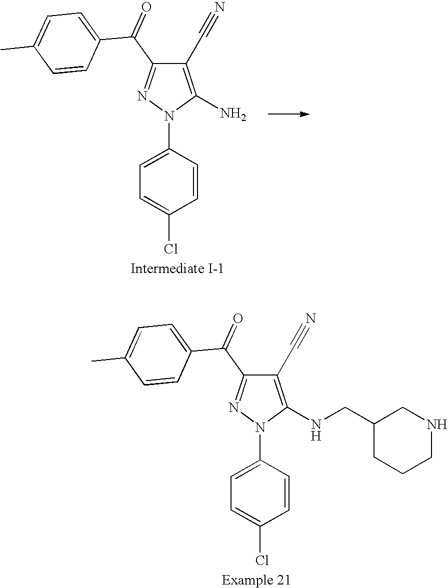

To Intermediate I-1 (229 mg, 0.682 mmol) was added N-Boc-3-methanesulfonyloxymethyl-piperidine (200 mg, 0.682 mmol), K₂CO₃ (188 mg, 1.36 mmol), and DMF (3 mL). The reaction was stirred under nitrogen at 100° C. for 16 h. The Boc-protected intermediate was isolated by aqueous workup. To the crude intermediate was added 30% TFA/DCM (3 mL), and the reaction was stirred under ambient conditions for 30 min. The deprotection was concentrated in vacuo and purified by reverse-phase HPLC (YMC-Pack Pro C18, 100×20 mm, 5 μm, gradient 20-100 ACN/H₂O with 0.1% TFA) to yield Example 21. LC-MS for C₂₄H₂₄ClN₅O [M+H⁺]: calculated 434.1, found 434.4.

### Example 22

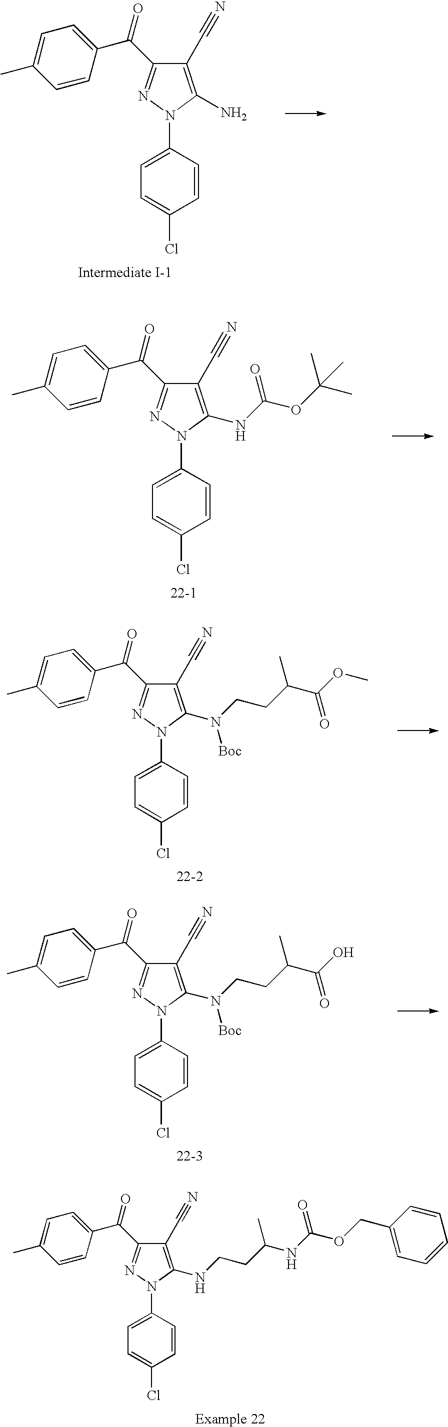

Step A: To Intermediate I-1 (10.85 g, 32.2 mmol) was added pyridine (75 mL) and di-tert-butyl carbonate (17.6 g, 80.5 mmol), and the reaction was stirred under ambient conditions for 2 h. The completed reaction was concentrated in vacuo. To the crude di-Boc intermediate was added THF/MeOH/H₂O (2:2:1, 170 mL) and LiOH.H₂O (4.06 g, 96.6 mmol), and the reaction was stirred under ambient conditions for 12 h. The product was isolated by aqueous workup to yield the compound 22-1. LCMS for C₂₃H₂₁ClN₄O₃ [M+H⁺]: calculated 437.2, found 437.8.
Step B: To compound 22-1 (456 mg, 1.05 mmol) was added K₂CO₃ (152 mg, 1.1 mmol) and DMF (5 mL). To the solution was added 4-chloro-2-methyl butyric acid methyl ester (3.7 mL, 2.2 mmol) and NaI (cat.), and the reaction was stirred under nitrogen at 70° C. for 18 h. The completed reaction was purified by column chromatoghraphy to yield compound 22-2. LCMS for C₂₉H₃₁ClN₄O₅ [M+H⁺]: calculated 551.2, found 495.2.
Step C. To a solution of compound 22-2 (100 mg, 0.181 mmol) in MeOH/THF/H₂O (4:4:1, 5 mL) was added LiOH.H₂O (100 mg, 2.4 mmol). The reaction was stirred under ambient conditions for 18 h. The completed reaction was purified by reverse phase HPLC (YMC-Pack Pro C18, 100×20 mm, 5 μm, gradient 20-100 CH₃CN/H₂O with 0.1% TFA) to yield compound 22-3. LCMS for C₂₈H₂₉ClN₄O₅ [M+H⁺]: calculated 537.2, found 481.2.
Step D. To a solution of compound 22-3 (80 mg, 0.149 mmol) in toluene (1 mL) was added diphenylphosphoryl azide (64 μL, 0.3 mmol) and triethylamine (42 μL, 0.3 mmol). The reaction was stirred under nitrogen at 60° C. for 2 h. To the reaction was added benzyl alcohol (2 mL), and the reaction was stirred under the same conditions an additional 20 h. The completed reaction was concentrated in vacuo. To the crude Boc-protected intermediate was added 30% TFA/DCM (2 mL), and the deprotection was stirred under ambient conditions for 1 h. The completed reaction was purified by prep TLC to yield Example 22. LCMS for C₃₀H₂₈ClN₅O₃ [M+H⁺]: calculated 542.2, found 542.3.

### Example 23

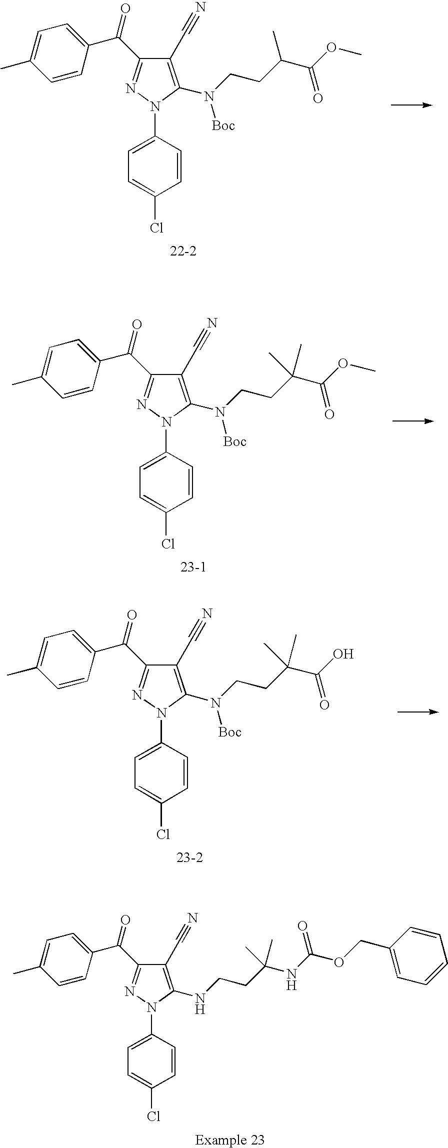

Step A: Under strict anhydrous conditions, to a solution of LiHMDS (0.5 M in THF, 1.6 mL) at −78° C. was added a solution of compound 22-2 (220 mg, 0.4 mmol). The reaction was stirred under nitrogen at −78° C. for 20 min. To the reaction was added methyl iodide (124 μL, 2.0 mmol). The reaction was stirred at ambient temperature under nitrogen for 18 h. The crude product was isolated by aqueous workup to yield compound 23-1. LCMS for C₃₀H₃₃ClN₄O₅ [M+H⁺]: calculated 565.2, found 565.8.
Step B: To a solution of compound 23-1 (120 mg, 0.212 mmol) in MeOH/THF/H₂O (4:4:1, 5 mL) was added LiOH.H₂O (100 mg, 2.4 mmol). The reaction was stirred under ambient conditions for 18 h. The completed reaction was purified by reverse phase HPLC (YMC-Pack Pro C18, 100×20 mm, 5 μm, gradient 20-100 CH₃CN/H₂O with 0.1% TFA) to yield compound 23-2. LCMS for C₂₉H₃₁ClN₄O₅ [M+H⁺]: calculated 551.2, found 481.2.
Step C: To a solution of compound 23-2 (109 mg, 0.198 mmol) in toluene (1 mL) was added diphenylphosphoryl azide (64 μL, 0.3 mmol) and triethylamine (42 μL, 0.3 mmol). The reaction was stirred under nitrogen at 60° C. for 2 h. To the reaction was added benzyl alcohol (2 mL), and the reaction was stirred under the same conditions for an additional 20 h. The completed reaction was concentrated in vacuo. To the crude Boc-protected intermediate was added 30% TFA/DCM (2 mL), and the deprotection was stirred under ambient conditions for 1 h. The completed reaction was purified by prep TLC to yield Example 23. LCMS for C₃₁H₃₀ClN₅O₃ [M+H⁺]: calculated 556.2, found 556.4.

### Example 24

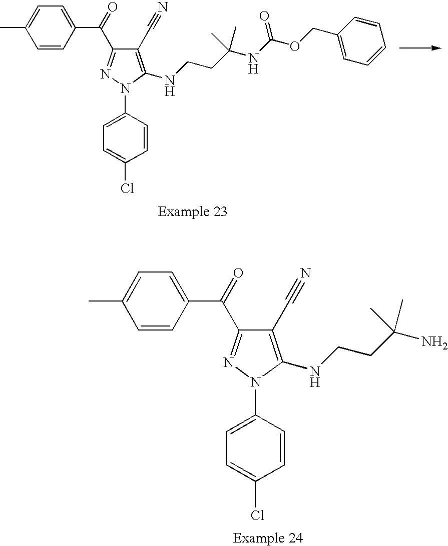

To a solution of Example 23 (20 mg) in EtOH (1 mL) was added 10% Pd/C (4 mg). The reaction was stirred under hydrogen (1 atm) for 7 min. The catalyst was removed by filtration through celite, and the filtrate was concentrated in vacuo. The crude product was purified by reverse phase HPLC (YMC-Pack Pro C₁₈, 100×20 mm, 5 μm, gradient 10-80 CH₃CN/H₂O with 0.1% TFA) to yield Example 24. LC-MS for C₂₃H₂₄ClN₅O [M+H⁺]: calculated 422.2, found 422.5.

The compound in Table 13 was prepared using the appropriate reagents following procedures similar to that described above for Example 24.

TABLE 13

| | | | Mass Found |
|---|---|---|---|
| Example | Structure | Parent Formula | [M + H] |
| 25 | 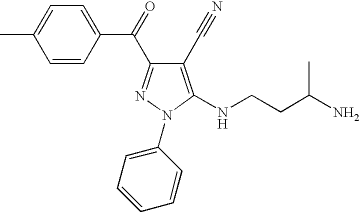 | C22H23N5O | 374.6 |

### Example 26

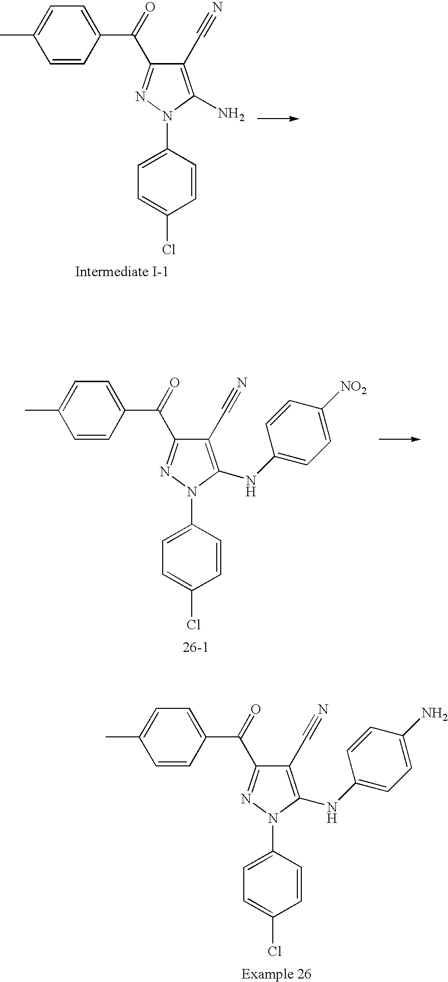

Step A: To Intermediate I-1 (200 mg, 0.595 mmol) was added 1-iodo-4-nitrobenzene (163 mg, 0.655 mmol), KOtBu (100 mg, 0.892 mmol), and 1,4-dioxane (3 mL). The reaction was stirred under nitrogen at 60° C. overnight. The product was isolated by an aqueous workup and purified by reverse-phase HPLC (YMC-Pack Pro C18, 100×20 mm, 5 μm, gradient 20-100 ACN/H₂O with 0.1% TFA) to yield compound 26-1. LC-MS for C₂₄H₁₆ClN₅O₃ [M+H⁺]: calculated 458.1, found 458.5.
Step B: To compound 26-1 (76 mg, 0.166 mmol) in ethanol (1 mL) was added a solution of SnCl₂ (157 mg, 0.831 mmol) in 10 N hydrochloric acid (0.3 mL). The reaction was stirred under nitrogen at 60° C. for 2 h. The product was isolated by an aqueous workup and purified by prep TLC (1000μ silica, 5% EtOAc/DCM) to yield Example 26. LC-MS for C₂₄H₁₂ClN₅O [M+H⁺]: calculated 428.1, found 428.4.

The compound in Table 14 was prepared using the appropriate reagents following procedures similar to that described above for Example 26.

TABLE 14

| | | | Mass Found |
|---|---|---|---|
| Example | Structure | Parent Formula | [M + H] |
| 27 | 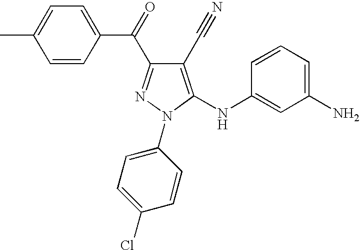 | C24H12ClN5O | 428.7 |

### Example 28

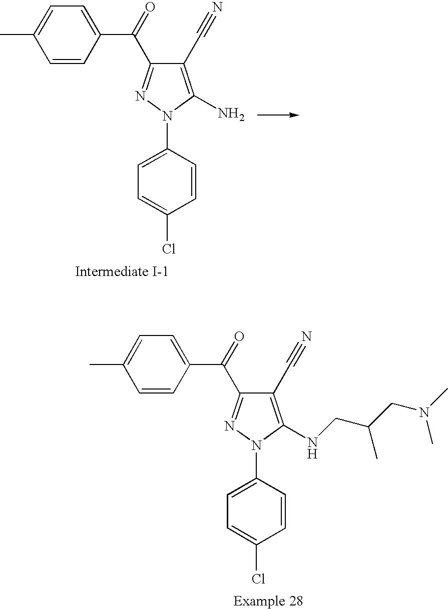

To Intermediate I-1 (100 mg, 0.298 mmol) was added (3-chloro-2-methylpropyl)-dimethylamine (102 mg, 0.596 mmol), K₂CO₃ (123 mg, 0.894 mmol), NaI (cat.), and DMF (1 mL). The reaction was heated at 100° C. under nitrogen for 6 h. The completed reaction was purified by reverse-phase HPLC (YMC-Pack Pro C18, 100×20 mm, 5 μm, gradient 20-90 ACN/H₂O with 0.1% TFA) to yield Example 28. LC-MS for C₂₄H₂₆ClN₅O [M+H⁺]: calculated 436.1, found 436.2.

The compounds in Table 15 were prepared using the appropriate reagents following procedures similar to that described above for Example 28.

TABLE 15

| | | | Mass Found |
|---|---|---|---|
| Example | Structure | Parent Formula | [M + H] |
| 29 | 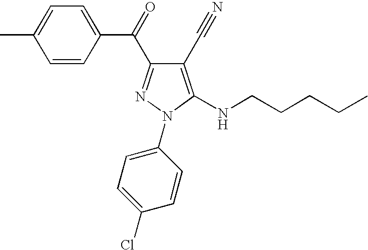 | C23H23ClN4O | 407.2 |
| 30 | 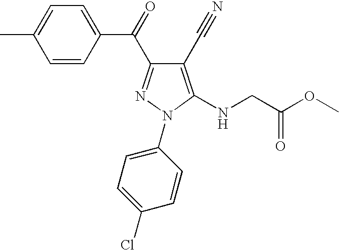 | C21H17ClN4O3 | 409.1 |
| 31 | 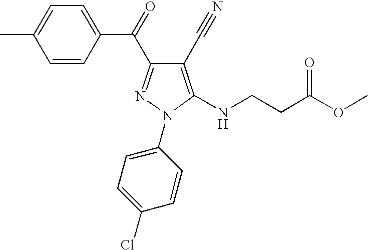 | C22H19ClN4O3 | 423.1 |
| 32 | 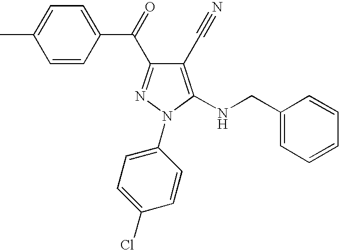 | C25H19ClN4O | 427.1 |
| 33 | 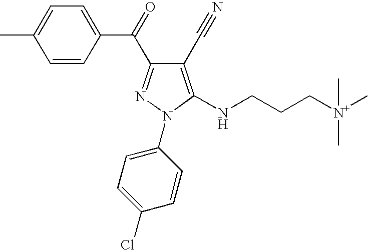 | C24H27ClN5O | 436.6 |
| 34 | 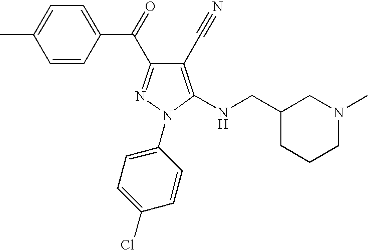 | C25H26ClN5O | 448.2 |
| 35 | 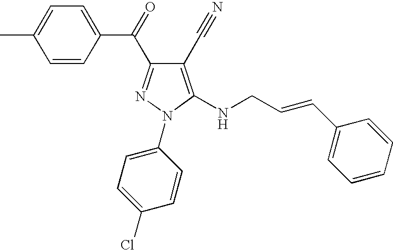 | C27H21ClN4O | 453.3 |
| 36 | 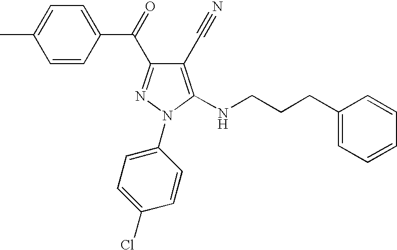 | C27H23ClN4O | 455.3 |
| 37 | 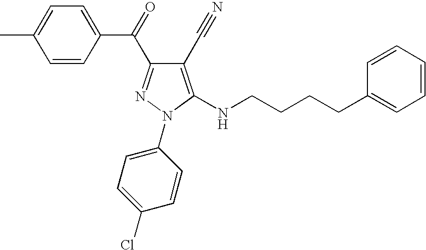 | C28H25ClN4O | 469.2 |
| 38 | 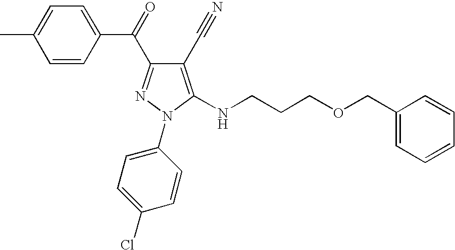 | C28H25ClN4O2 | 485.2 |
| 39 | 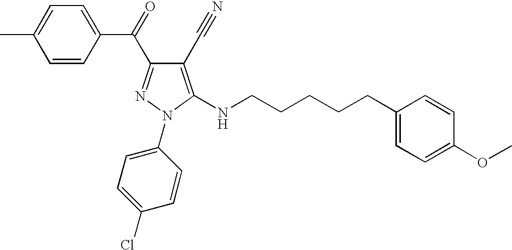 | C30H29ClN4O2 | 513.3 |
| 40 | 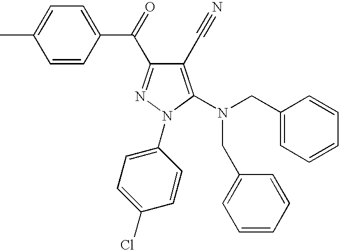 | C32H25ClN4O | 517.1 |
| 41 | 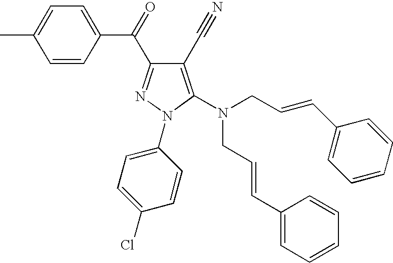 | C36H29ClN4O | 569.4 |

### Example 42

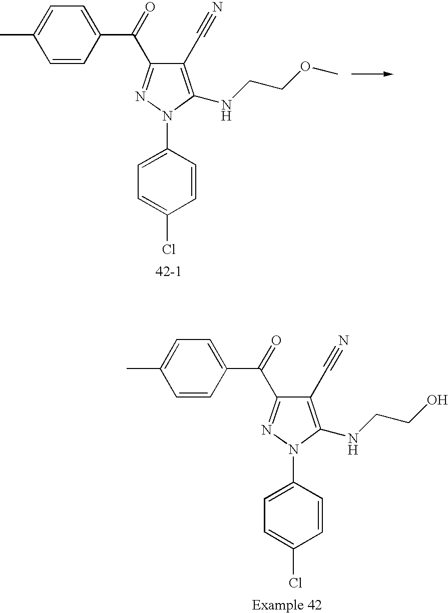

Step A: Compound 42-1 may be prepared according to the procedure outlined for Example 28 using the appropriate reagents.
Step B: Under strict anhydrous conditions, to a solution of Compound 42-1 (53 mg, 0.133 mmol) in DCM (3.5 mL) at −78° C. was added BBr₃ (1 M in DCM, 1.33 mL). The reaction was slowly warmed to 0° C. and stirred at that temperature for 3 h. The crude product was isolated by aqueous workup and purified by column chromatography to yield Example 42. ¹H NMR (500 MHz, d6-DMSO) δ 8.04 (d, 2H), 7.65 (s, 4H), 7.33 (d, 2H), 6.79 (t, 1H), 4.82 (t, 1H), 3.58 (m, 2H), 3.49 (m, 2H), 2.37 (s, 3H).

### Example 43

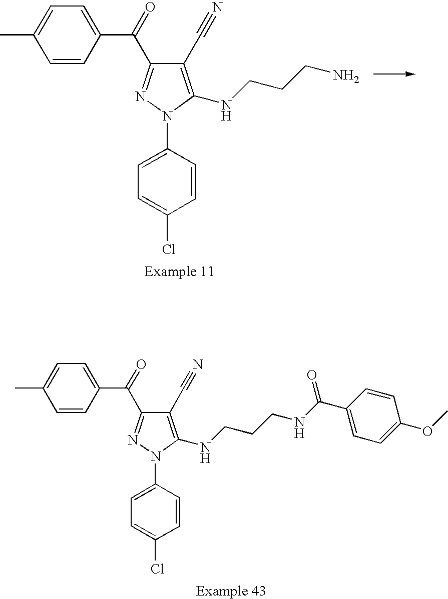

To Example 11 (801 mg, 2.03 mmol) was added p-anisic acid (371.2 mg, 2.44 mmol), EDAC.HCl coupling reagent (585 mg, 3.05 mmol), and DCM (10 mL). The reaction was stirred overnight under ambient conditions. The completed reaction was concentrated in vacuo and purified by reverse-phase HPLC (YMC-Pack Pro C18, 100×20 mm, 5 μm, gradient 20-100 ACN/H₂O with 0.1% TFA) to yield Example 43. ¹H NMR (500 MHz, d6-DMSO) δ 8.34 (t, 1H), 8.03 (d, 2H), 7.78 (m, 2H), 7.64 (s, 4H), 7.32 (d, 2H), 6.96 (m, 3H), 3.79 (s, 3H) 3.47 (m, 2H), 3.31 (m, 2H), 2.37 (s, 3H), 1.84 (m, 2H).

The compounds in Table 16 were prepared using the appropriate reagents following procedures similar to that described above for Examples 42 and 43.

TABLE 16

| | | | Mass |
|---|---|---|---|
| | | | Found |
| Example | Structure | Parent Formula | [M + H] |
| 44 | 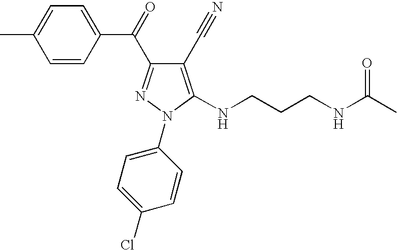 | C23H22ClN5O2 | 436.5 |
| 45 | 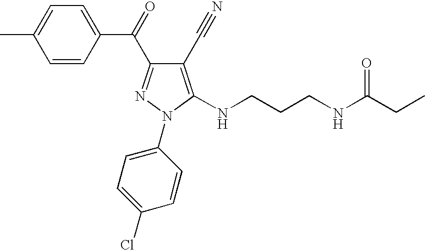 | C24H24ClN5O2 | 450.5 |
| 46 | 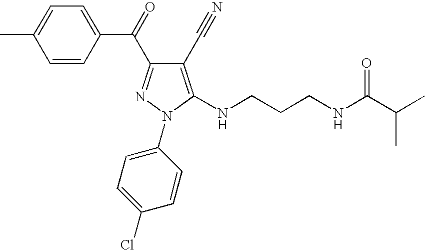 | C25H26ClN5O2 | 464.3 |
| 47 | 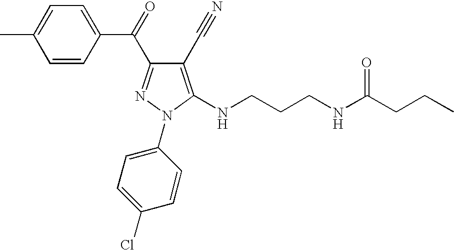 | C25H26ClN5O2 | 464.5 |
| 48 | 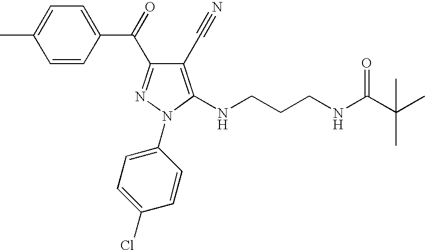 | C26H28ClN5O2 | 478.4 |
| 49 | 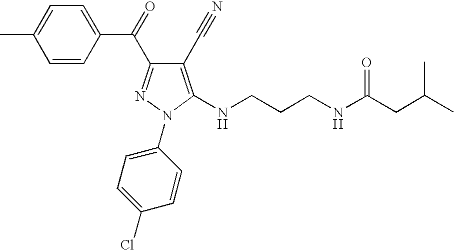 | C26H28ClN5O2 | 478.5 |
| 50 | 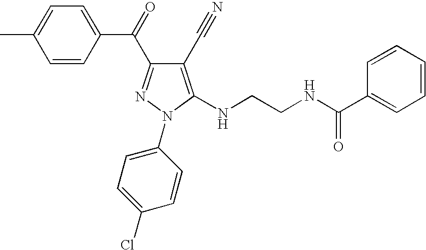 | C27H22ClN5O2 | 484.3 |
| 51 | 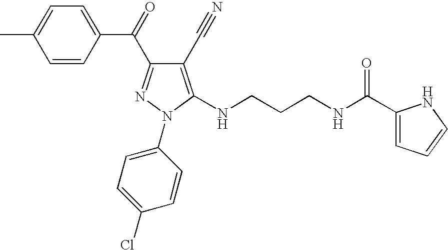 | C26H23ClN6O2 | 487.4 |
| 52 | 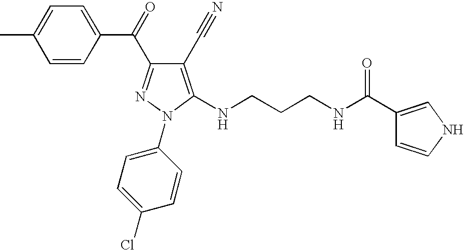 | C26H23ClN6O2 | 487.5 |
| 53 | 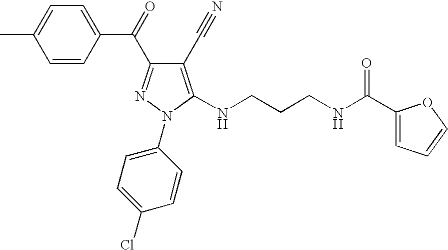 | C26H22ClN5O3 | 488.2 |
| 54 | 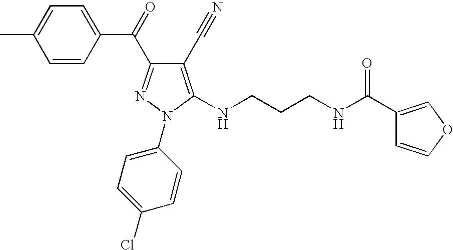 | C26H22ClN5O3 | 488.4 |
| 55 | 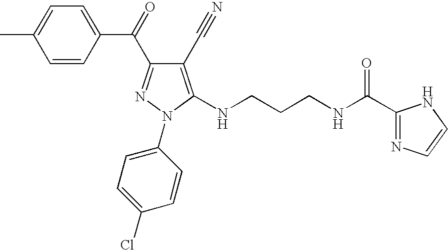 | C25H22ClN7O2 | 488.7 |
| 56 | 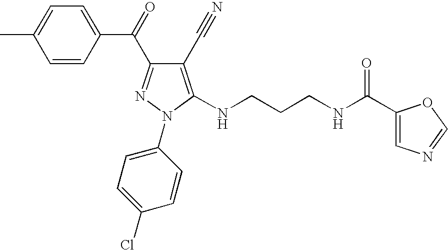 | C25H21ClN6O3 | 489.2 |
| 57 | 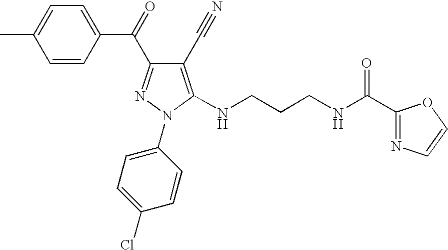 | C25H21ClN6O3 | 489.2 |
| 58 | 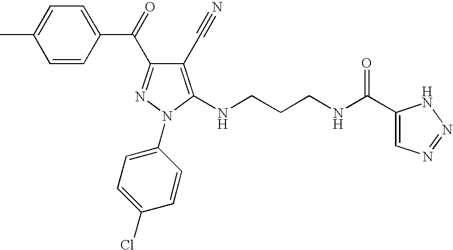 | C24H21ClN8O2 | 489.2 |
| 59 | 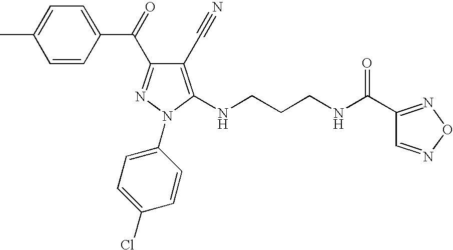 | C24H20ClN7O3 | 490.2 |
| 60 | 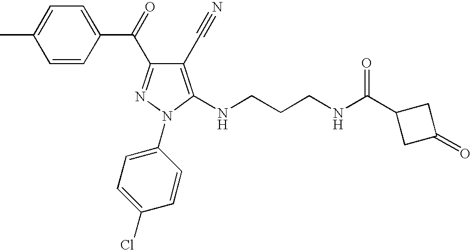 | C26H24ClN5O3 | 490.2 |
| 61 | 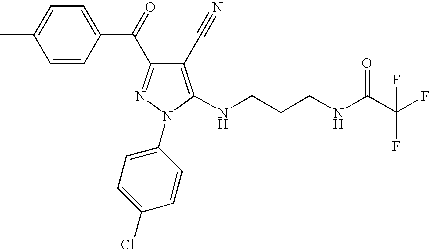 | C23H19ClF3N5O2 | 490.5 |
| 62 | 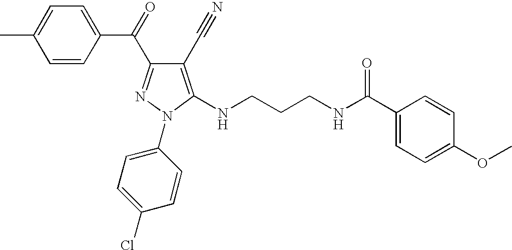 | C24H25ClN6O4 | 497.2 |
| 63 | 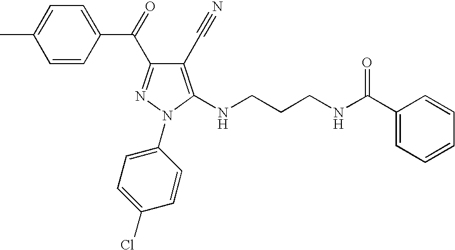 | C28H24ClN5O2 | 498.3 |
| 64 | 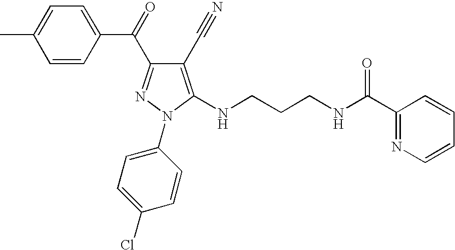 | C27H23ClN6O2 | 499.3 |
| 65 | 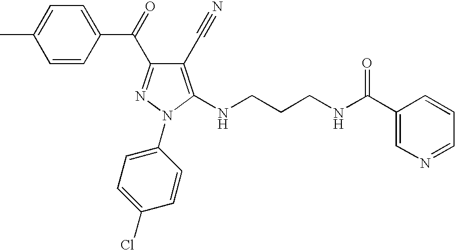 | C27H23ClN6O2 | 499.3 |
| 66 | 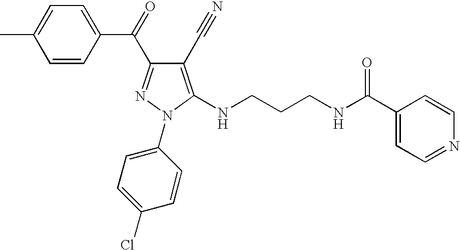 | C27H23ClN6O2 | 499.3 |
| 67 | 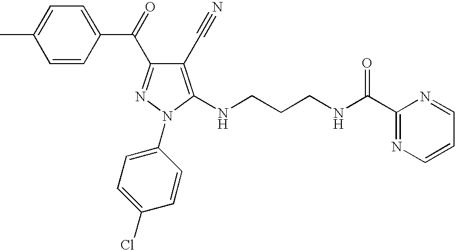 | C26H22ClN7O2 | 500.2 |
| 68 | 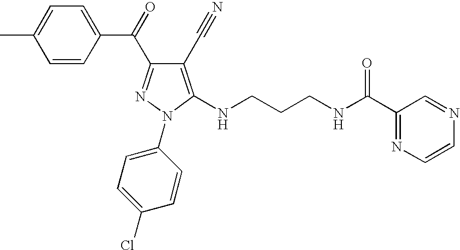 | C26H22ClN7O2 | 500.2 |
| 69 | 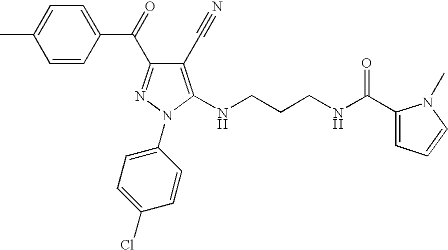 | C27H25ClN6O2 | 501.5 |
| 70 | 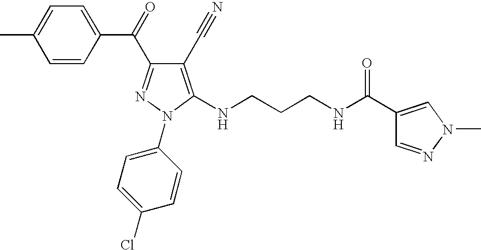 | C26H24ClN7O2 | 502.7 |
| 71 | 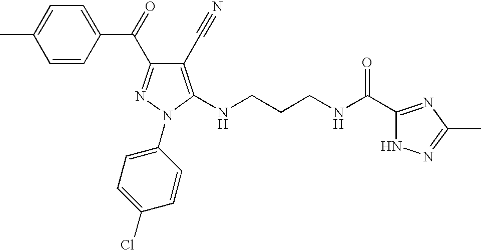 | C25H23ClN8O2 | 503.3 |
| 72 | 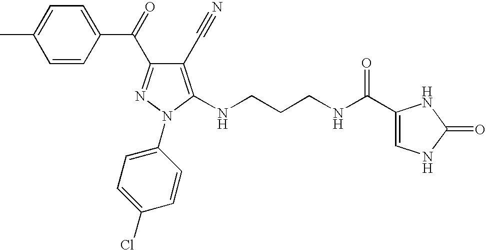 | C25H22ClN7O3 | 504.2 |
| 73 | 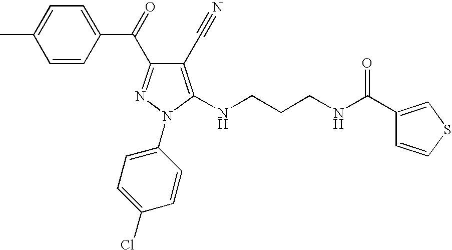 | C26H22ClN5O2S | 504.2 |
| 74 | 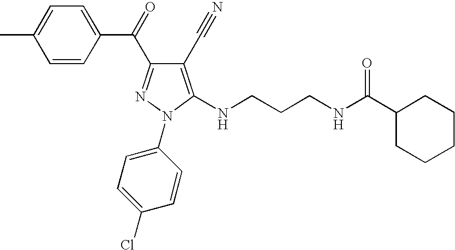 | C28H30ClN5O2 | 504.4 |
| 75 | 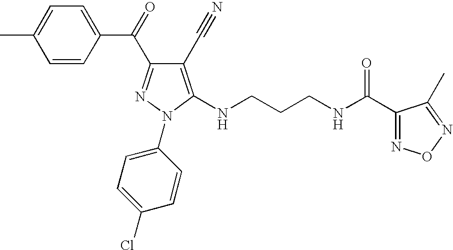 | C25H22ClN7O3 | 504.6 |
| 76 | 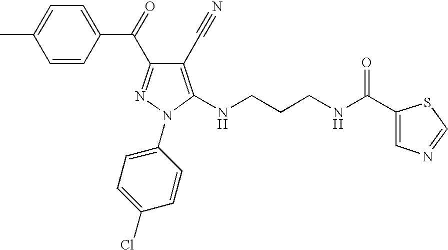 | C25H21ClN6O2S | 505.6 |
| 77 | 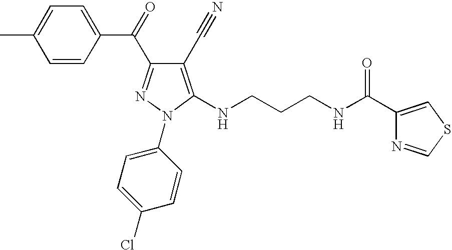 | C25H21ClN6O2S | 505.6 |
| 78 | 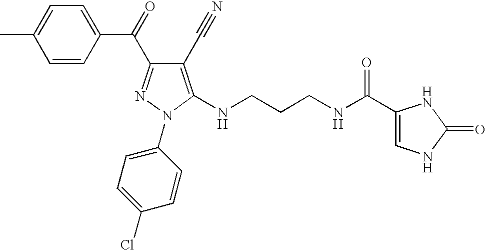 | C24H21ClN8O3 | 505.7 |
| 79 | 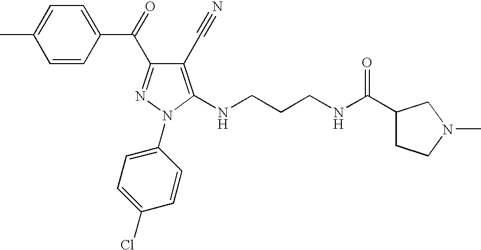 | C27H29ClN6O2 | 505.7 |
| 80 | 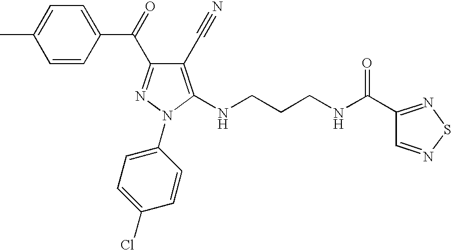 | C24H20ClN7O2S | 506.2 |
| 81 | 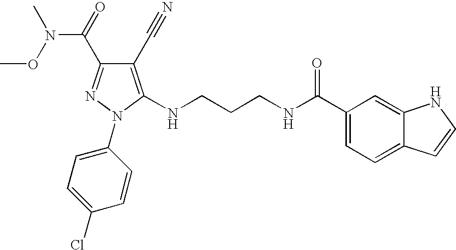 | C25H24ClN7O3 | 506.3 |
| 82 | 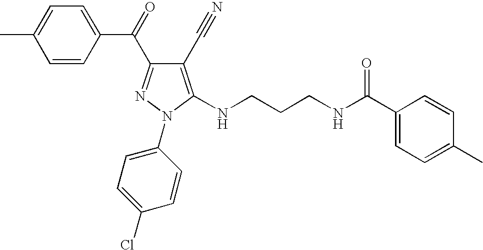 | C29H26ClN5O2 | 512.2 |
| 83 | 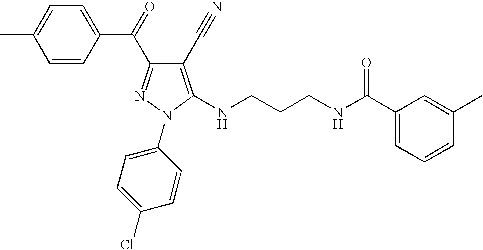 | C29H26ClN5O2 | 512.3 |
| 84 | 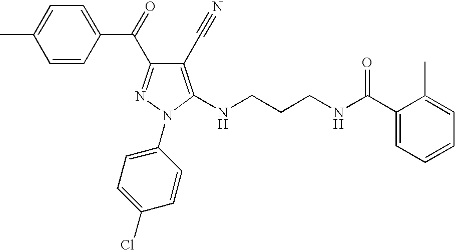 | C29H26ClN5O2 | 512.3 |
| 85 | 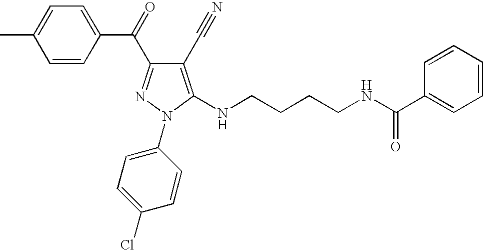 | C29H26ClN5O2 | 512.3 |
| 86 | 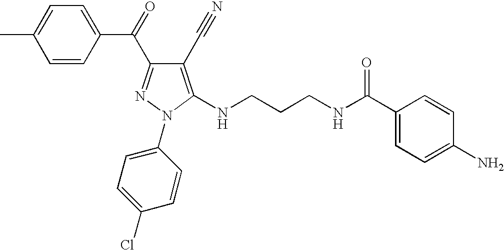 | C28H25ClN6O2 | 513.2 |
| 87 | 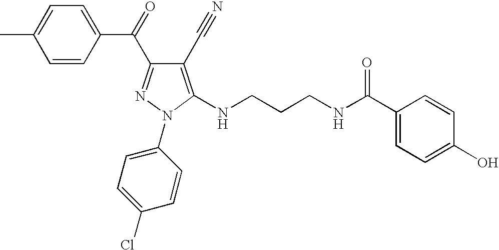 | C28H24ClN5O3 | 514.5 |
| 88 | 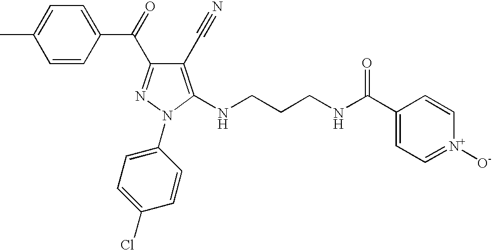 | C27H23ClN6o3 | 515.3 |
| 89 | 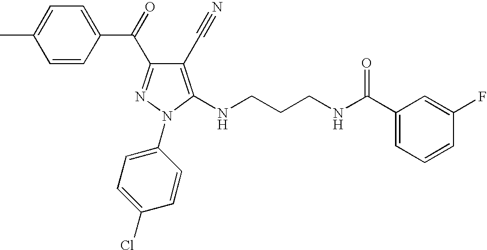 | C28H23ClFN5O2 | 516.2 |
| 90 | 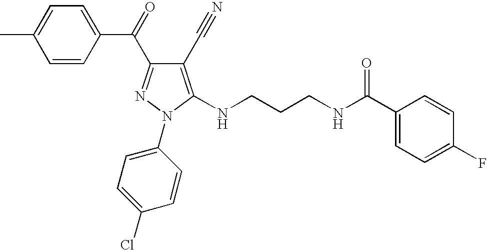 | C28H23ClFN5O2 | 516.3 |
| 91 | 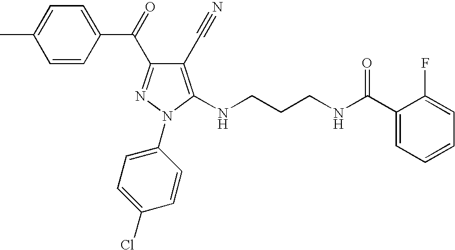 | C28H23ClFN5O2 | 516.3 |
| 99 | 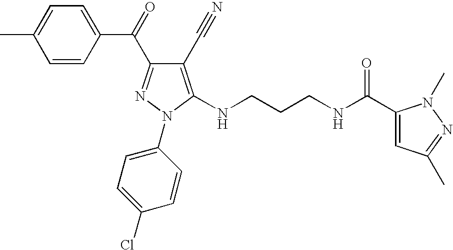 | C27H26ClN7O2 | 516.3 |
| 100 | 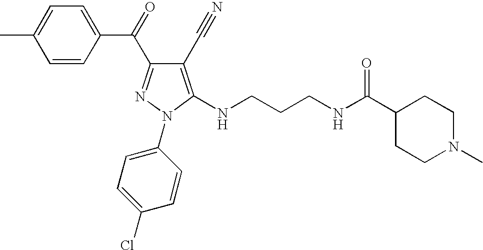 | C28H31ClN6O2 | 519.3 |
| 101 | 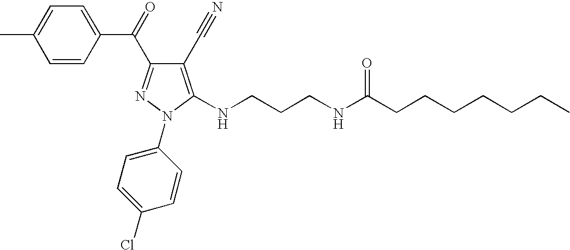 | C29H34ClN5O2 | 520.6 |
| 102 | 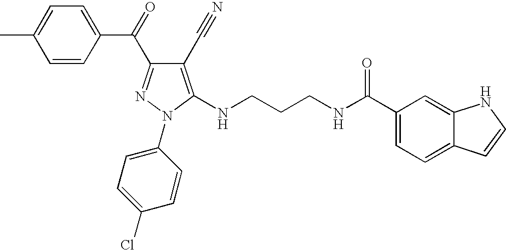 | C30H25FN6O2 | 521.9 |
| 103 | 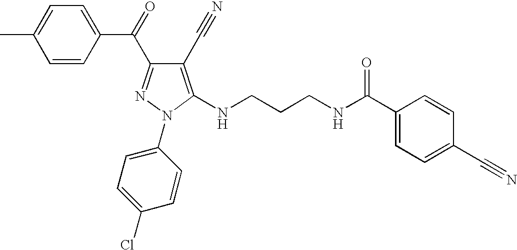 | C29H23ClN6O2 | 523.2 |
| 104 | 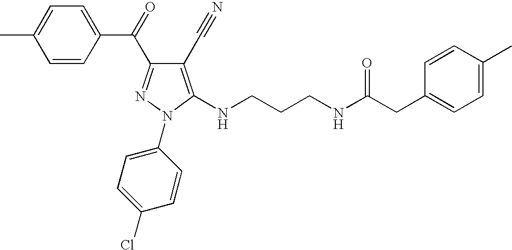 | C30H28ClN5O2 | 526.3 |
| 105 | 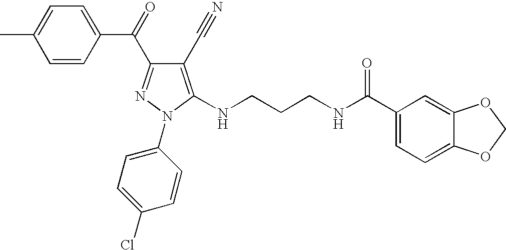 | C29H24FN5O4 | 526.5 |
| 106 | 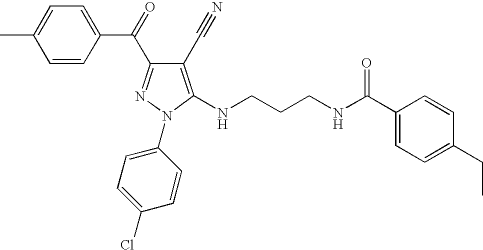 | C30H28ClN5O2 | 526.5 |
| 107 | 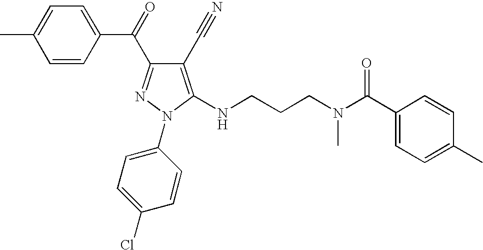 | C30H28ClN5O2 | 526.9 |
| 108 | 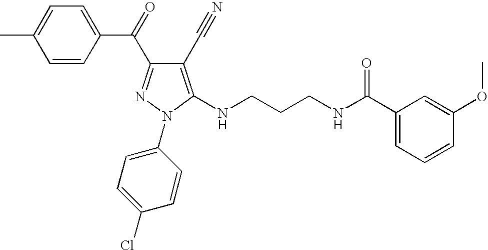 | C29H26ClN5O3 | 528.3 |
| 109 | 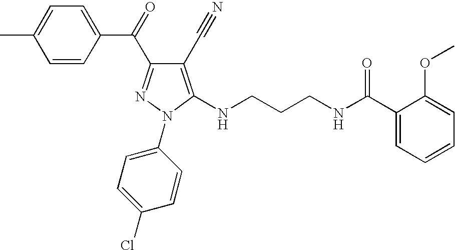 | C29H26ClN5O3 | 528.3 |
| 110 | 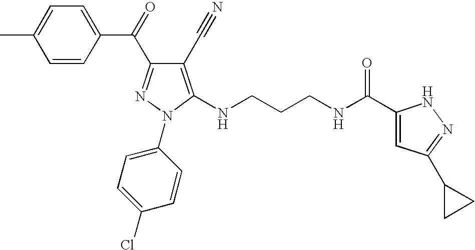 | C28H26ClN7O2 | 528.8 |
| 111 | 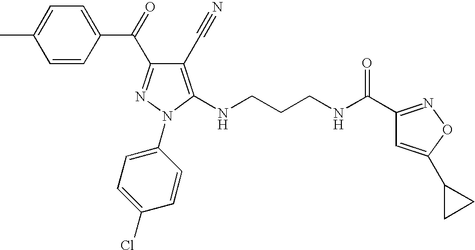 | C28H25ClN6O3 | 529.3 |
| 112 | 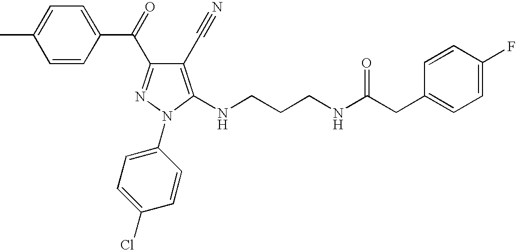 | C29H25ClFN5O2 | 530.2 |
| 113 | 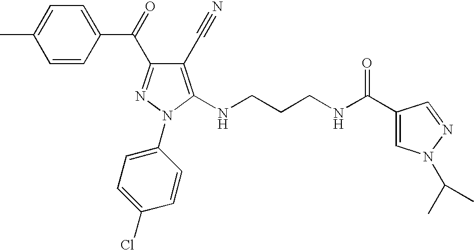 | C28H28ClN7O2 | 530.3 |
| 114 | 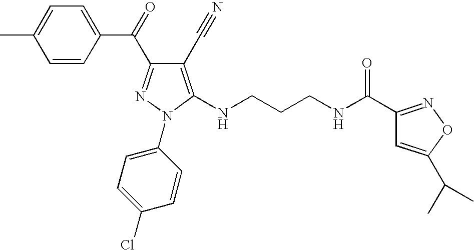 | C28H27ClN6O3 | 531.7 |
| 115 | 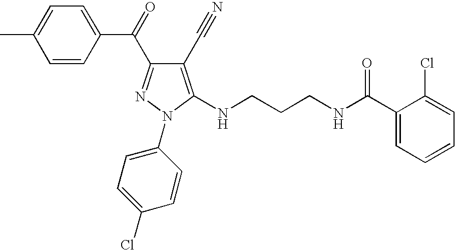 | C28H23C12N5O2 | 532.3 |
| 116 | 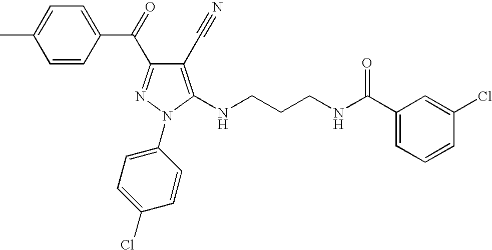 | C28H23C12N5O2 | 532.3 |
| 117 | 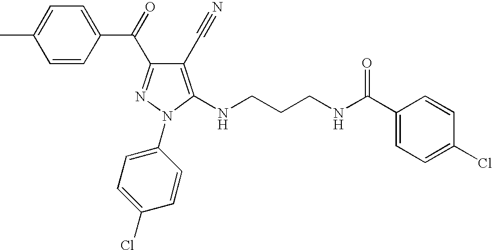 | C28H23C12N5O2 | 532.3 |
| 118 | 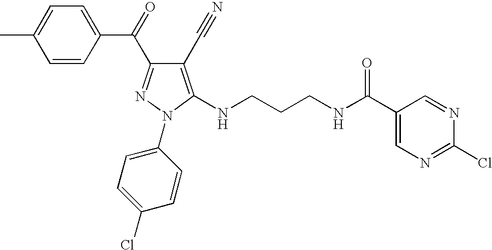 | C26H21CI2N7O2 | 534.2 |
| 119 | 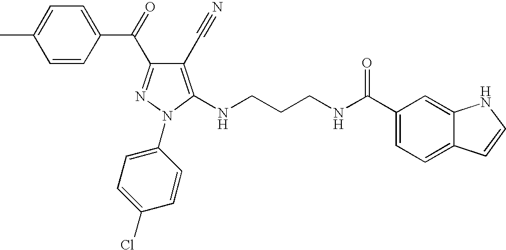 | C30H25ClN6O2 | 537.2 |
| 120 | 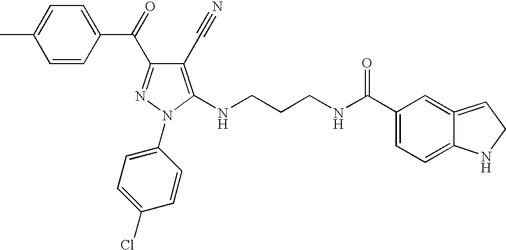 | C30H25ClN6O2 | 537.3 |
| 121 | 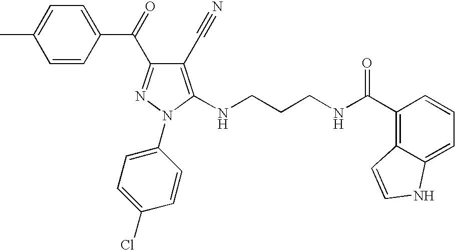 | C30H25ClN6O2 | 537.3 |
| 122 | 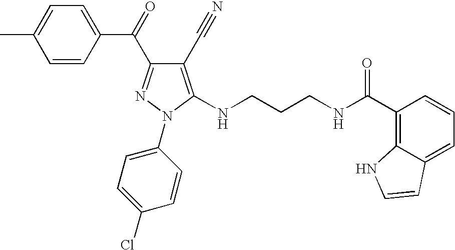 | C30H25ClN6O2 | 537.3 |
| 123 | 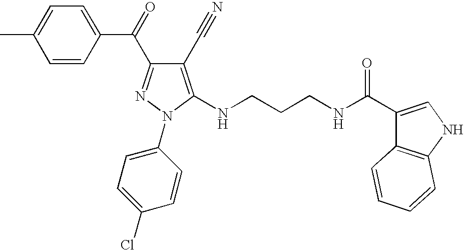 | C30H25ClN6O2 | 537.4 |
| 124 | 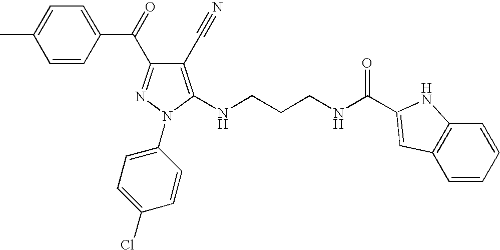 | C30H25ClN6O2 | 537.5 |
| 125 | 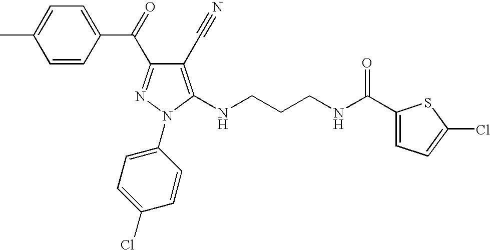 | C26H21C12N5O2S | 538.1 |
| 126 | 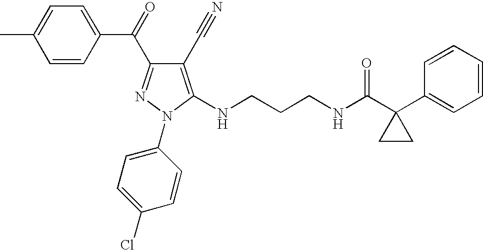 | C31H28ClN5O2 | 538.2 |
| 127 | 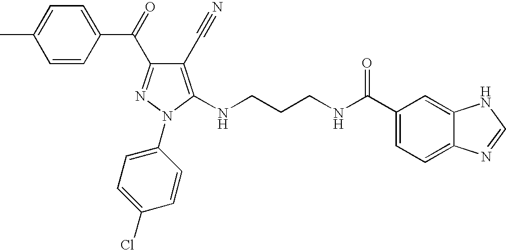 | C29H24ClN7O2 | 538.3 |
| 128 | 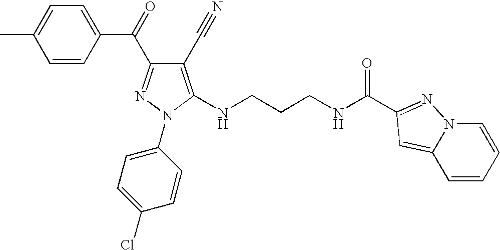 | C29H24ClN7O2 | 538.3 |
| 129 | 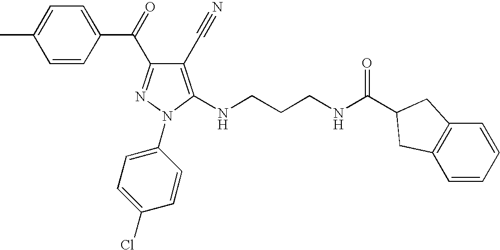 | C31H28ClN5O2 | 538.3 |
| 130 | 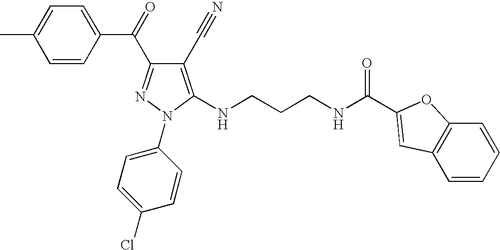 | C30H24ClN5O3 | 538.4 |
| 131 | 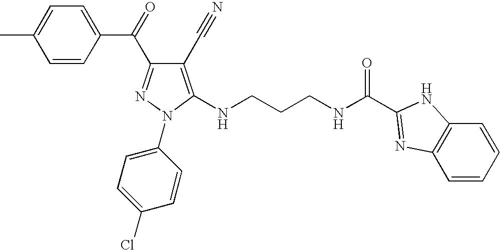 | C29H24ClN7O2 | 538.4 |
| 132 | 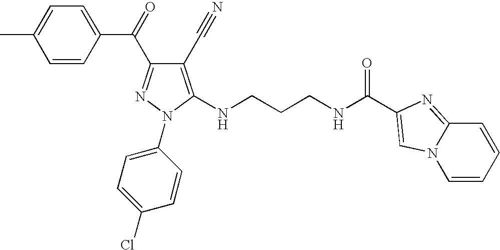 | C29H24ClN7O2 | 538.7 |
| 133 | 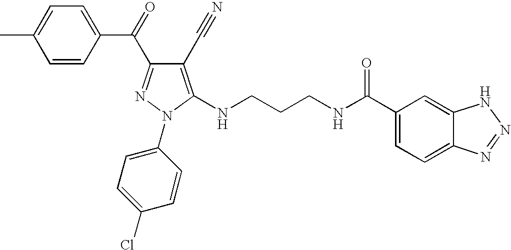 | C28H23ClN8O2 | 539.4 |
| 134 | 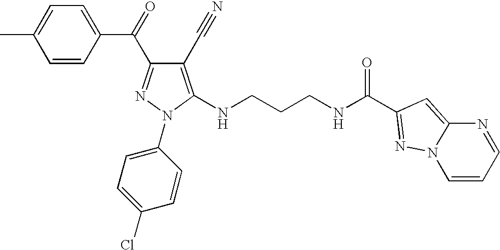 | C28H23ClN8O2 | 539.7 |
| 135 | 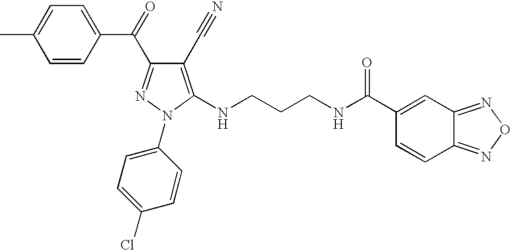 | C28H22ClN7O3 | 540.3 |
| 136 | 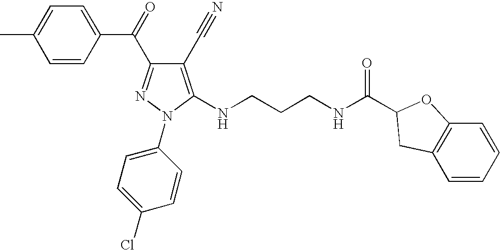 | C30H26ClN5O3 | 540.3 |
| 137 | 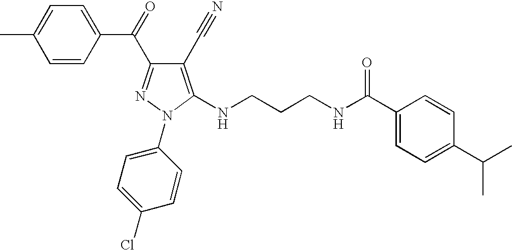 | C31H30ClN5O2 | 540.3 |
| 138 | 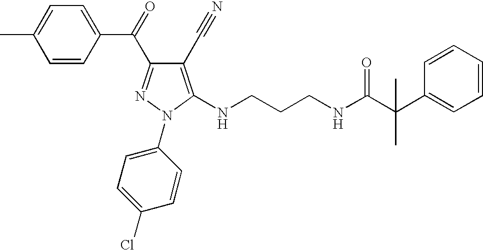 | C31H30ClN5O2 | 540.6 |
| 139 | 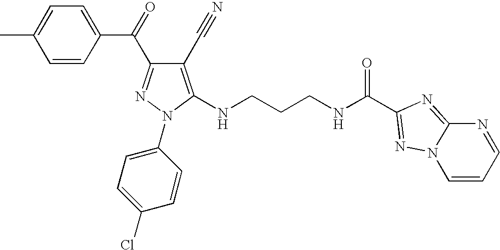 | C27H22ClN902 | 540.7 |
| 140 | 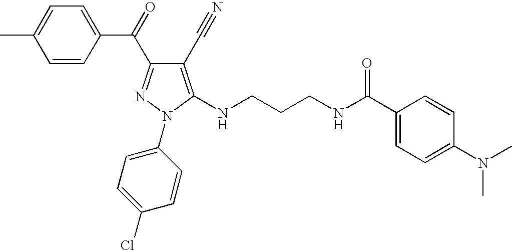 | C30H29ClN6O2 | 541.3 |
| 141 | 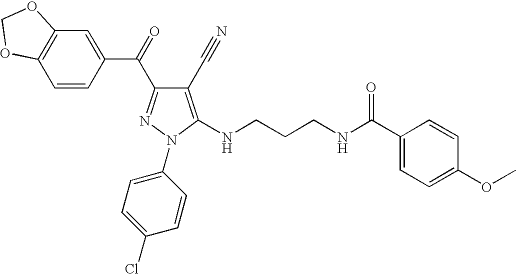 | C29H24FN5O5 | 542.3 |
| 142 | 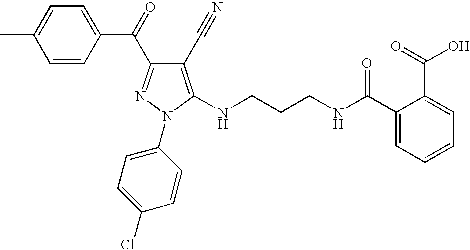 | C29H24ClN5O4 | 542.3 |
| 143 | 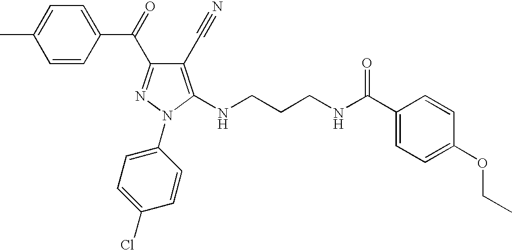 | C30H28ClN5O3 | 542.3 |
| 144 | 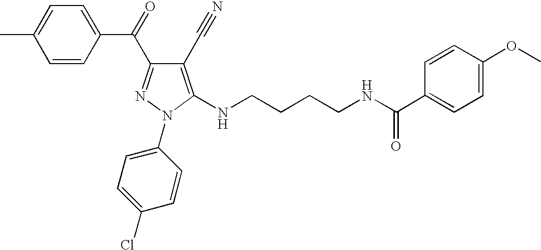 | C30H28ClN5O3 | 542.3 |
| 145 | 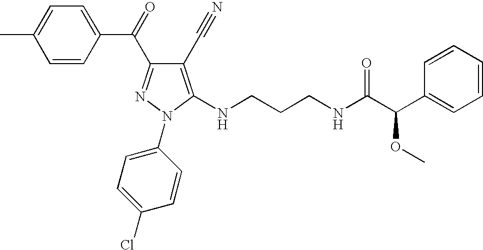 | C30H28ClN5O3 | 542.3 |
| 146 | 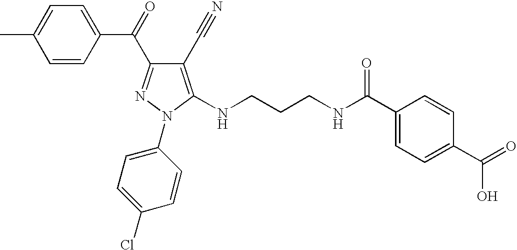 | C29H24ClN5O4 | 542.4 |
| 147 | 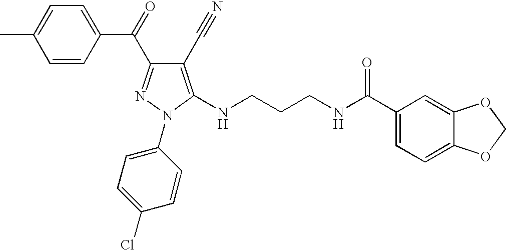 | C29H24ClN5O4 | 542.5 |
| 148 | 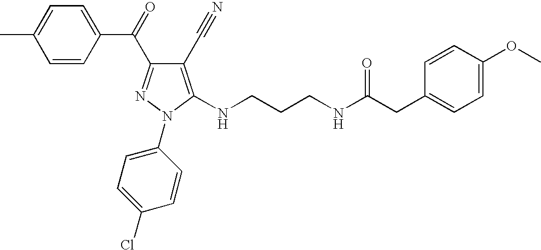 | C30H28ClN5O3 | 542.6 |
| 149 | 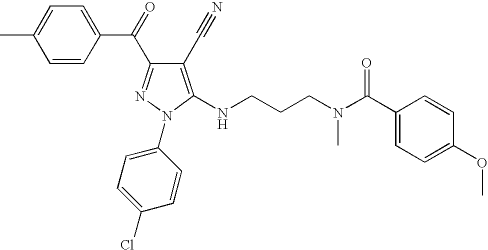 | C30H28ClN5O3 | 542.9 |
| 150 | 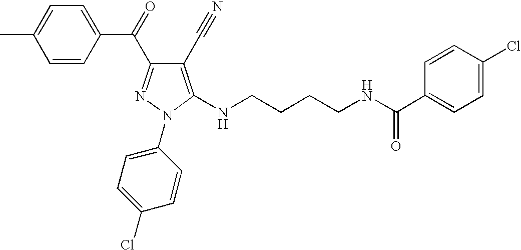 | C29H25Cl2N5O2 | 546.2 |
| 151 | 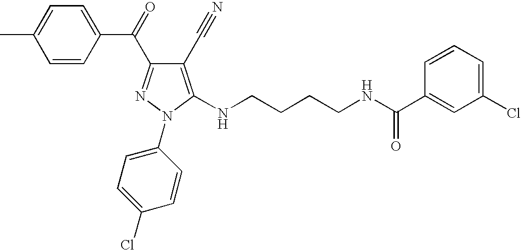 | C29H25Cl2N5O2 | 546.2 |
| 152 | 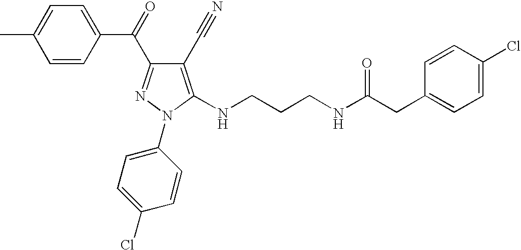 | C29H25Cl2N5O2 | 546.2 |
| 153 | 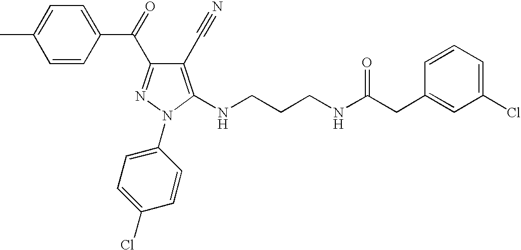 | C29H25Cl2N5O2 | 546.2 |
| 154 | 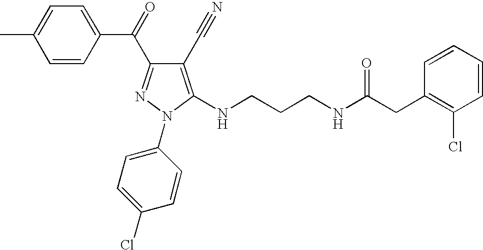 | C29H25Cl2N5O2 | 546.2 |
| 155 | 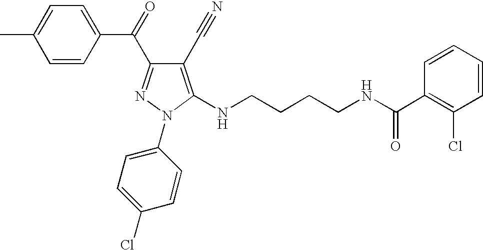 | C29H25Cl2N5O2 | 546.5 |
| 156 | 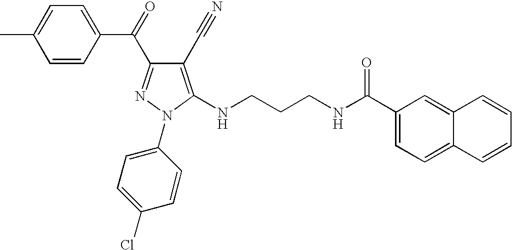 | C32H26ClN5O2 | 548.4 |
| 157 | 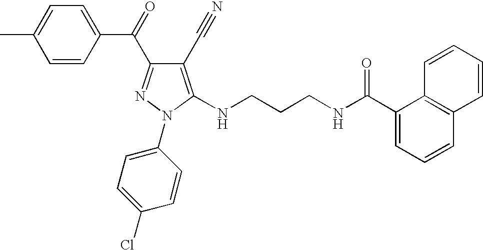 | C32H26ClN5O2 | 548.5 |
| 158 | 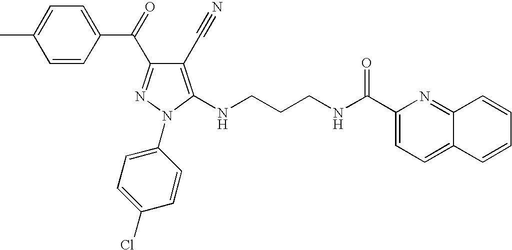 | C31H25ClN6O2 | 549.4 |
| 159 | 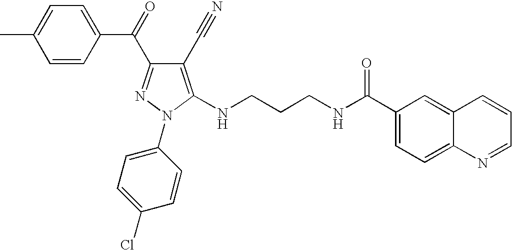 | C31H25ClN6O2 | 549.5 |
| 160 | 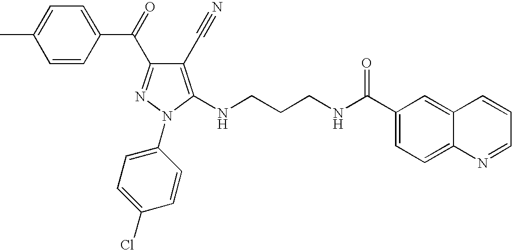 | C31H25ClN6O2 | 549.5 |
| 161 | 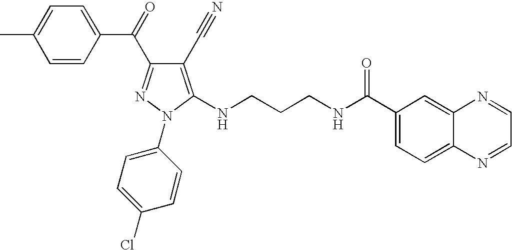 | C30H24ClN7O2 | 550.3 |
| 162 | 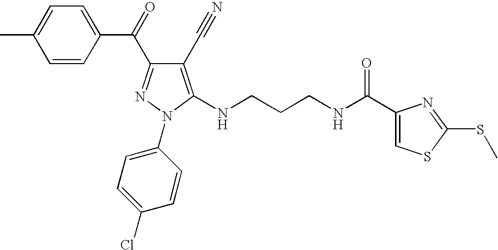 | C26H23ClN6O2S2 | 551.2 |
| 163 | 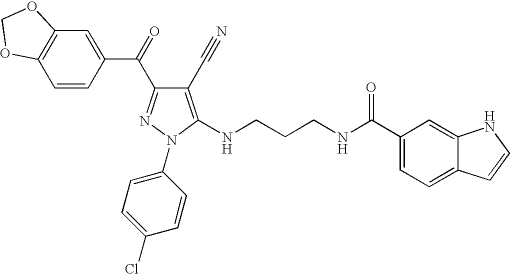 | C30H23FN6O4 | 551.4 |
| 164 | 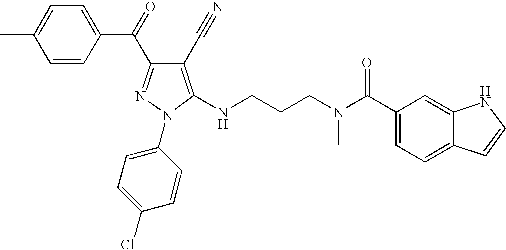 | C31H27ClN6O2 | 551.8 |
| 165 | 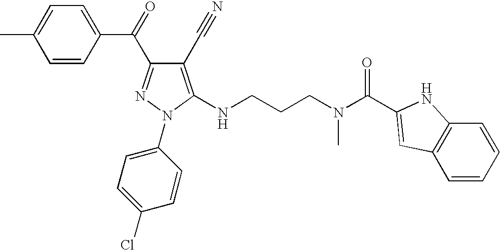 | C31H27ClN6O2 | 551.8 |
| 166 | 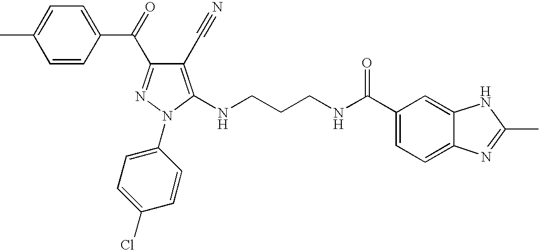 | C30H26ClN7O2 | 552.3 |
| 167 | 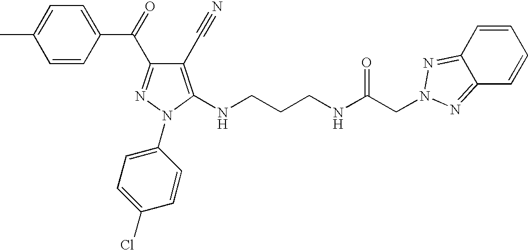 | C29H25ClN8O2 | 553.3 |
| 168 | 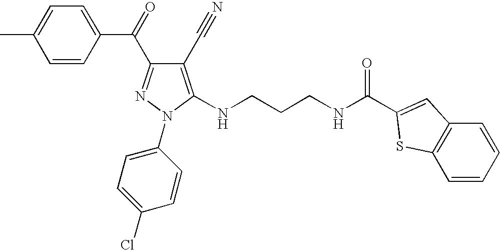 | C30H24ClN5O2S | 554.3 |
| 169 | 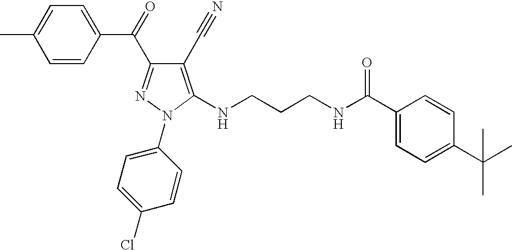 | C32H32ClN5O2 | 554.3 |
| 170 | 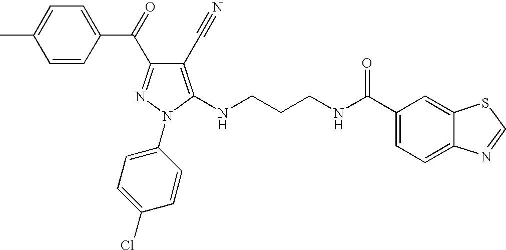 | C29H23ClN6O2S | 555.2 |
| 171 | 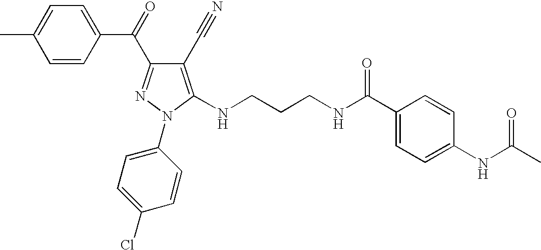 | C30H27ClN6O3 | 555.6 |
| 172 | 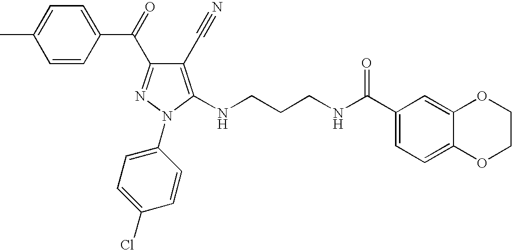 | C30H26ClN5O4 | 556.2 |
| 173 | 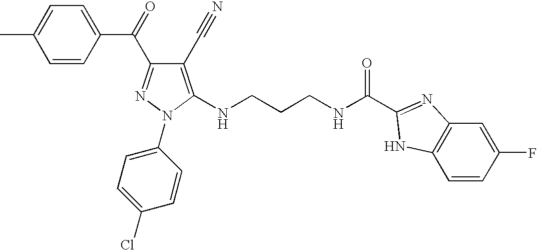 | C29H23ClFN7O2 | 556.4 |
| 174 | 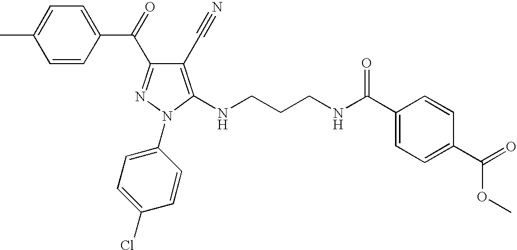 | C30H26ClN5O4 | 556.5 |
| 175 | 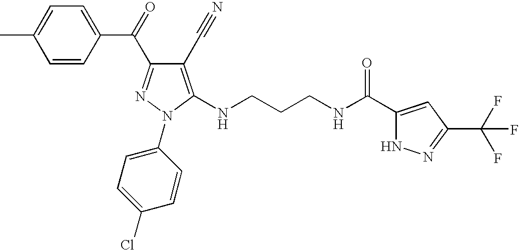 | C26H2101F3N7O2 | 556.7 |
| 176 | 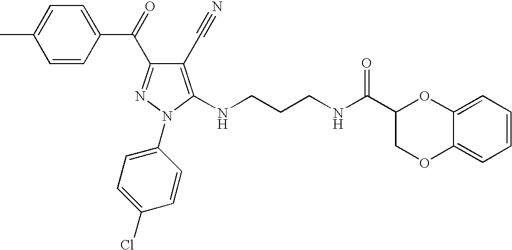 | C30H26ClN5O4 | 556.7 |
| 177 | 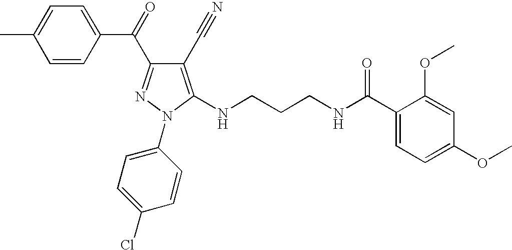 | C30H28ClN5O4 | 558.3 |
| 178 | 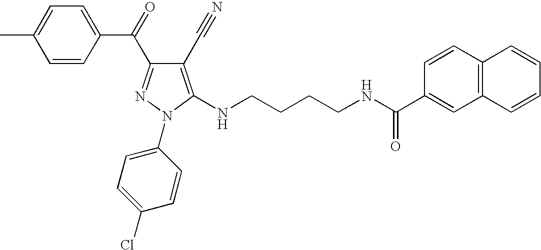 | C33H28ClN5O2 | 562.3 |
| 179 | 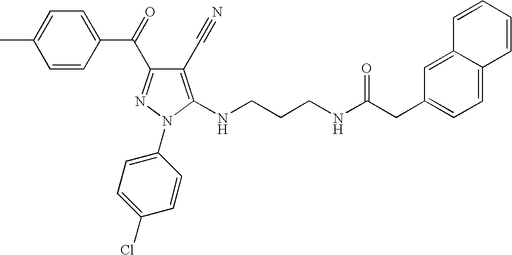 | C33H28ClN5O2 | 562.3 |
| 180 | 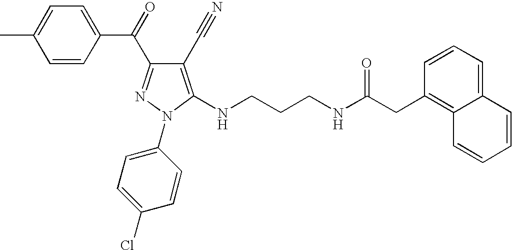 | C33H28ClN5O2 | 562.5 |
| 181 | 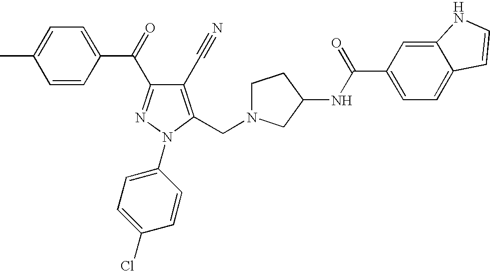 | C32H27ClN6O2 | 564.1 |
| 182 | 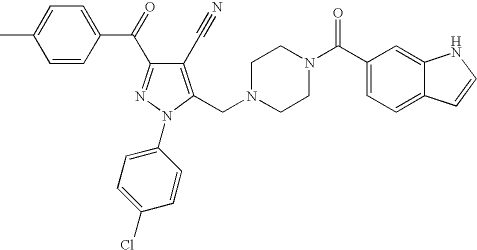 | C32H27ClN6O2 | 564.1 |
| 183 | 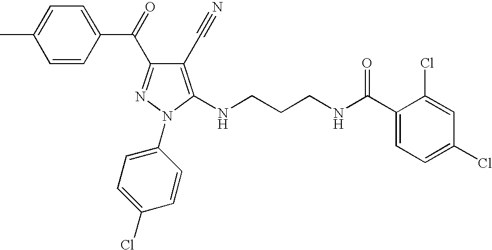 | C28H22C13N5O2 | 566.1 |
| 184 | 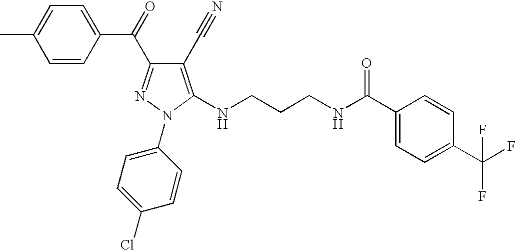 | C29H23ClF3N5O2 | 566.2 |
| 185 | 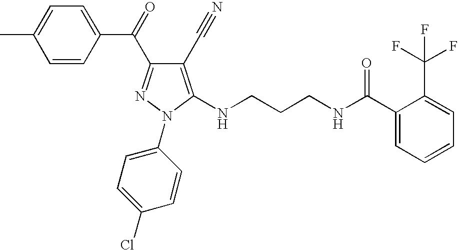 | C29H23ClF3N5O2 | 566.2 |
| 186 | 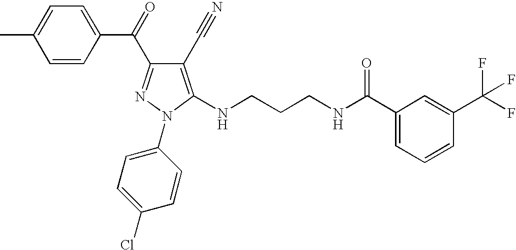 | C29H23ClF3N5O2 | 566.3 |
| 187 | 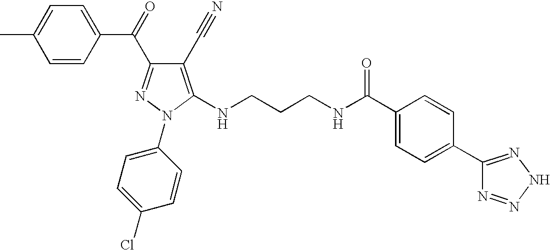 | C29H24ClN9O2 | 566.6 |
| 188 | 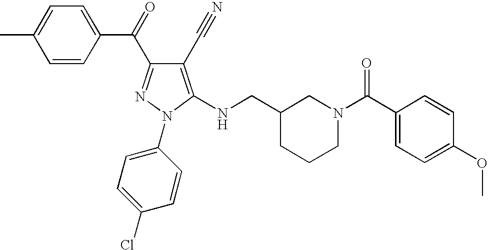 | C32H30ClN5O3 | 568.3 |
| 189 | 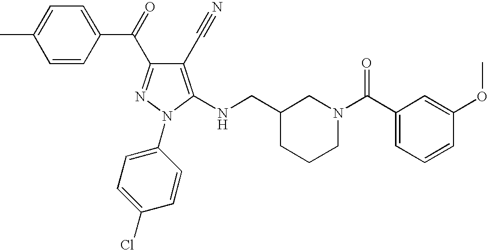 | C32H30ClN5O3 | 568.3 |
| 190 | 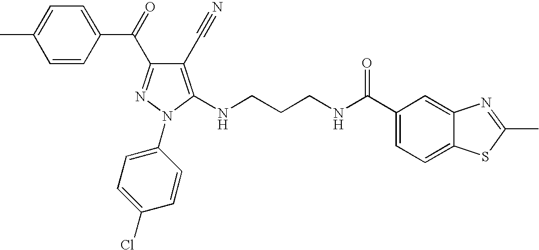 | C30H25ClN6O2S | 569.3 |
| 191 | 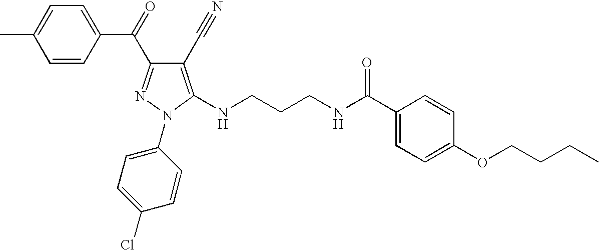 | C32H32ClN5O3 | 570.3 |
| 192 | 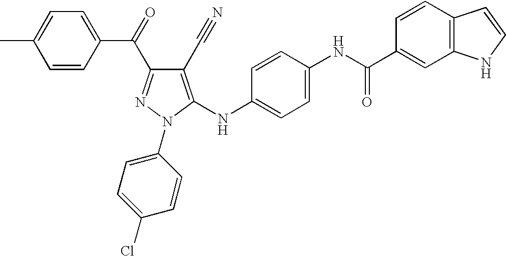 | C33H23ClN6O2 | 571.6 |
| 193 | 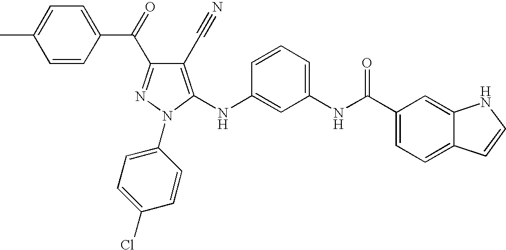 | C33H23ClN6O2 | 571.6 |
| 194 | 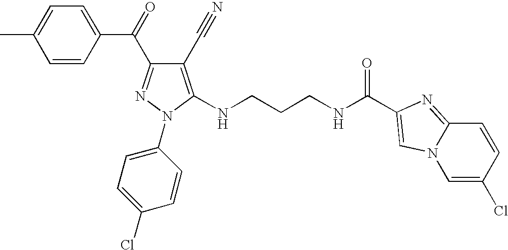 | C29H23Cl2N7O2 | 572.3 |
| 195 | 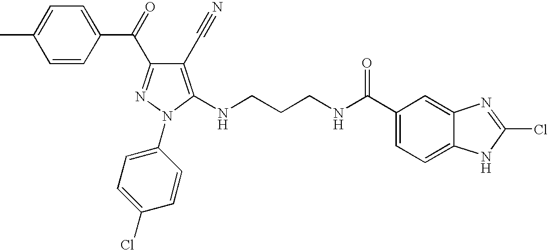 | C29H23Cl2N7O2 | 572.3 |
| 196 | 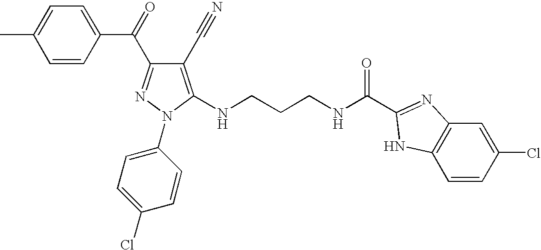 | C29H23012N7O2 | 572.4 |
| 197 | 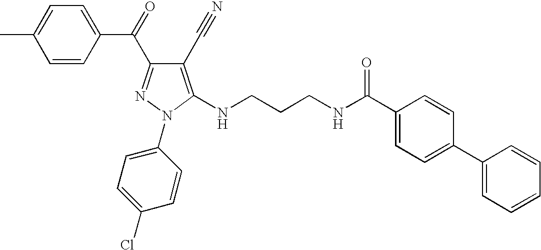 | C34H28ClN5O2 | 5743 |
| 198 | 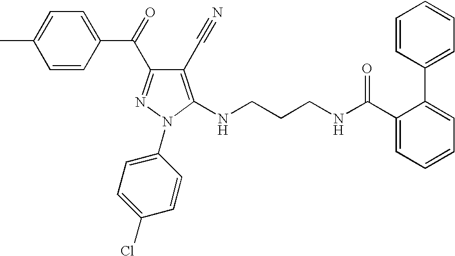 | C34H28ClN5O2 | 574.6 |
| 199 | 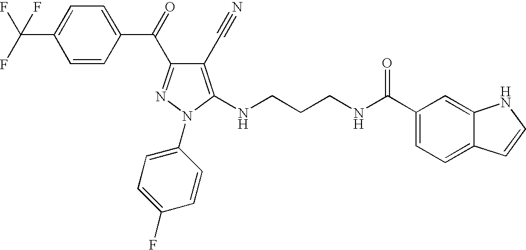 | C30H22F4N6O2 | 575.9 |
| 200 | 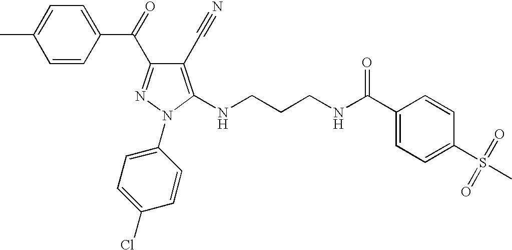 | C29H26ClN5O4S | 576.2 |
| 201 | 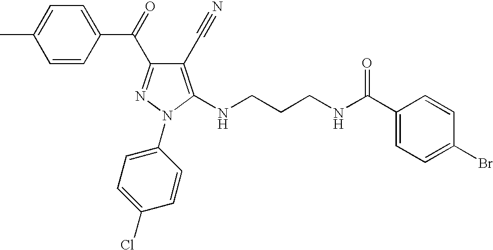 | C28H23BrClN5O2 | 576.2 |
| 202 | 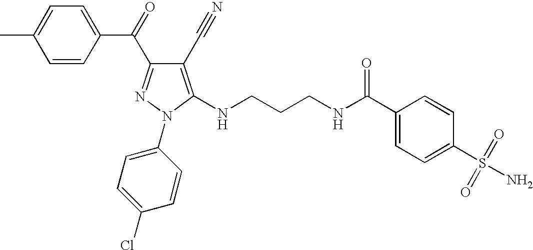 | C28H25ClN6O4S | 577.2 |
| 203 | 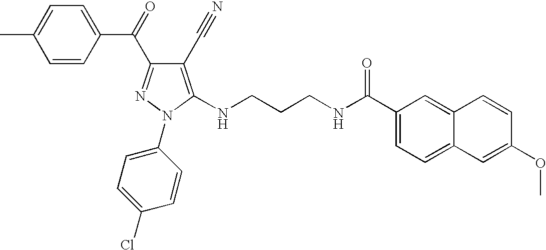 | C33H28ClN5O3 | 578.5 |
| 204 | 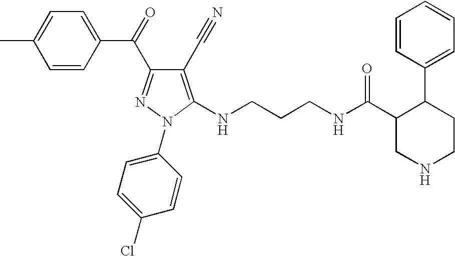 | C33H33ClN6O2 | 581.7 |
| 205 | 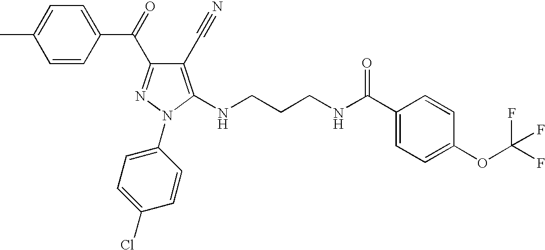 | C29H23ClF3N5O3 | 582.2 |
| 206 | 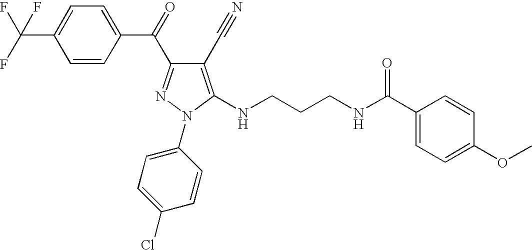 | C29H23ClF3N5O3 | 582.3 |
| 207 | 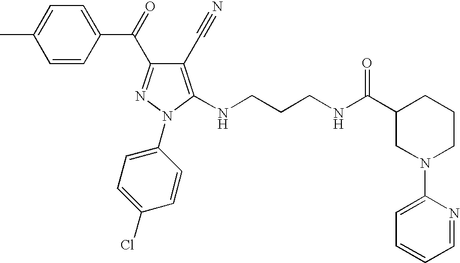 | C32H32ClN7O2 | 582.3 |
| 208 | 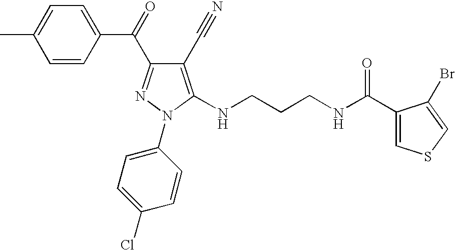 | C26H21BrClN5O2S | 582.6 |
| 209 | 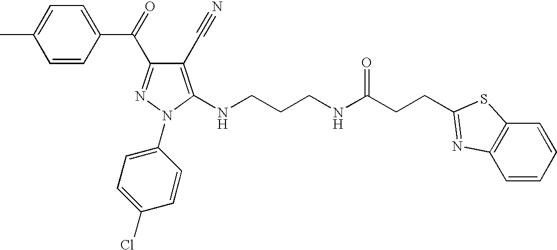 | C31H27ClN6O2S | 583.3 |
| 210 | 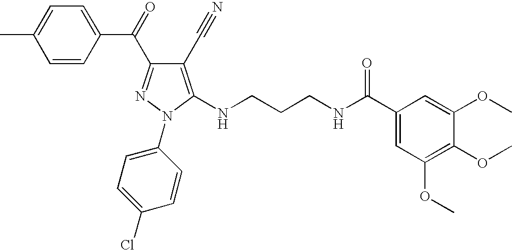 | C31H30ClN5O5 | 588.2 |
| 211 | 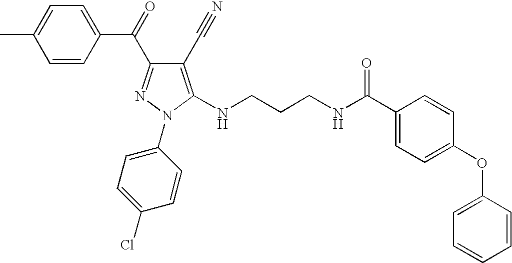 | C34H28ClN5O3 | 590.3 |
| 212 | 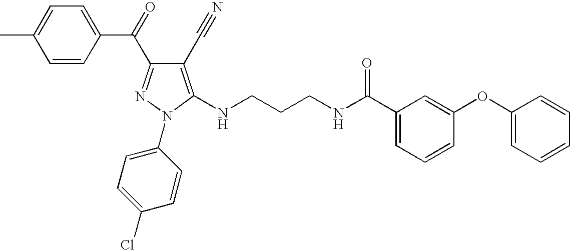 | C34H28ClN5O3 | 590.3 |
| 213 | 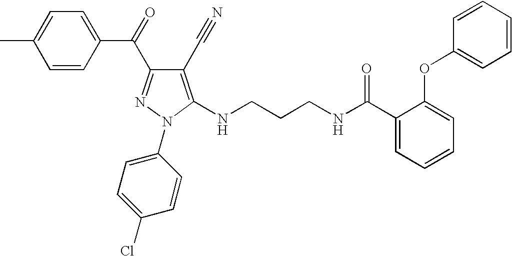 | C34H28ClN5O3 | 590.3 |
| 214 | 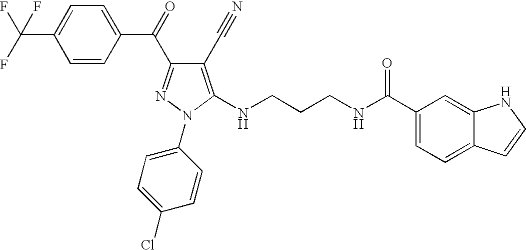 | C30H22ClF3N6O2 | 591.8 |
| 215 | 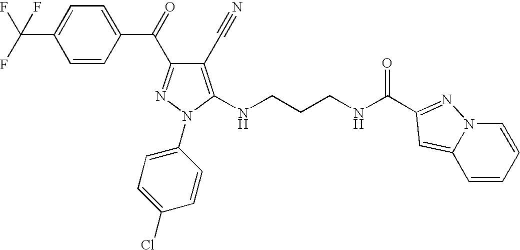 | C29H21ClF3N7O2 | 592.2 |
| 216 | 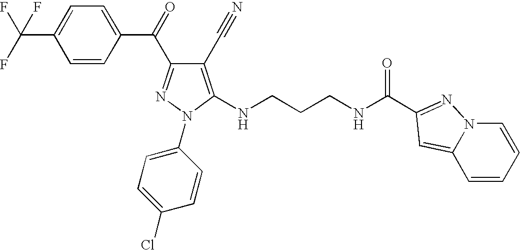 | C29H21ClF3N7O2 | 592.5 |
| 217 | 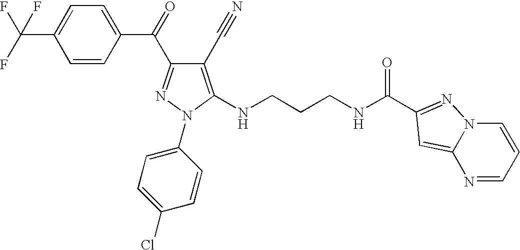 | C28H20ClF3N8O2 | 593.5 |
| 218 | 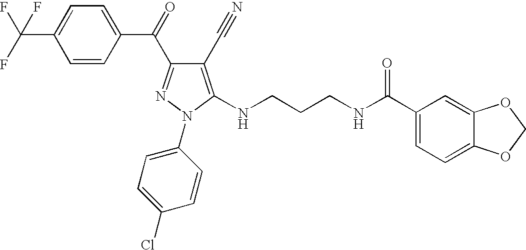 | C29H21ClF3N5O4 | 596.5 |
| 219 | 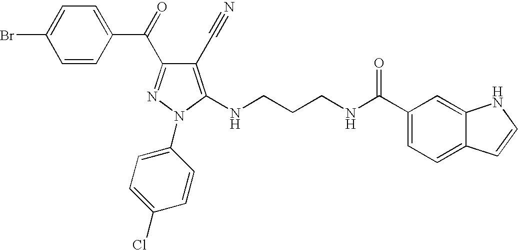 | C29H22BrClN6O2 | 601.7 |
| 220 | 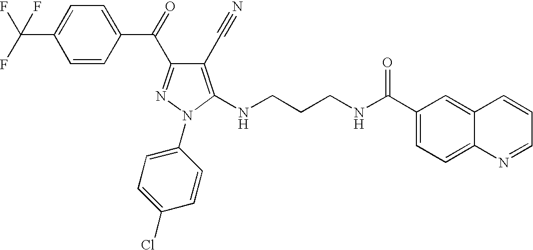 | C31H22ClF3N6O2 | 603.5 |
| 221 | 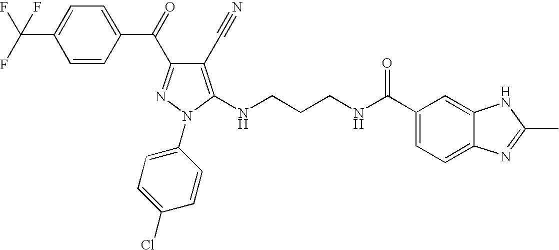 | C30H23ClF3N7O2 | 606.1 |
| 222 | 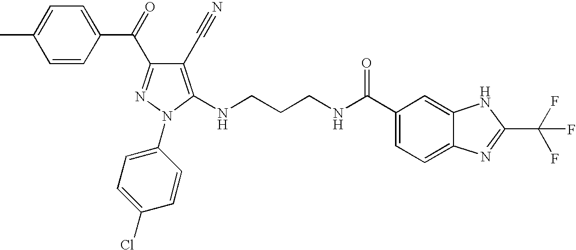 | C30H23ClF3N7O2 | 606.4 |
| 223 | 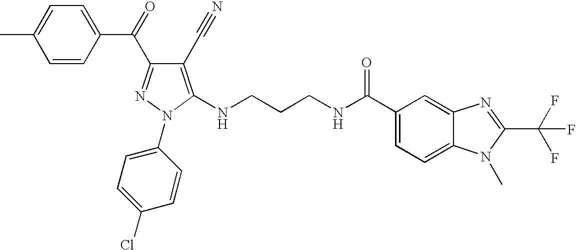 | C31H25ClF3N7O2 | 620.4 |
| 224 | 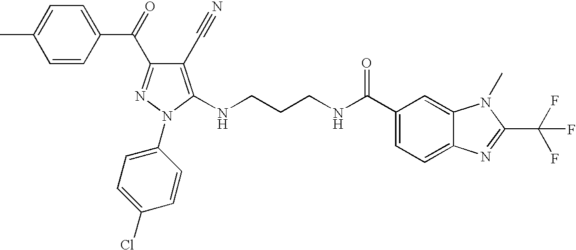 | C31H25ClF3N7O2 | 620.4 |
| 225 | 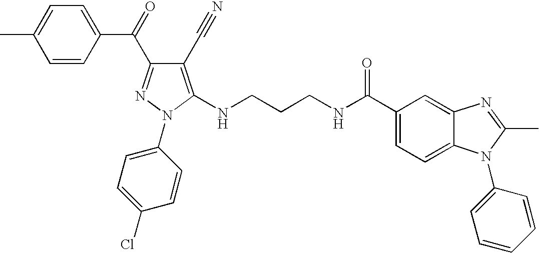 | C36H30ClN7O2 | 628.4 |
| 226 | 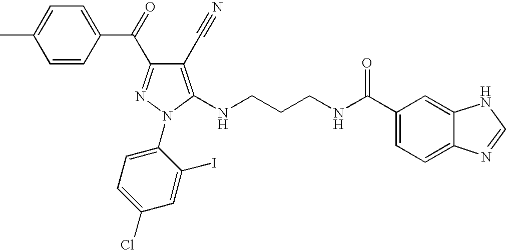 | C30H24ClIN6O2 | 663.2 |
| 227 | 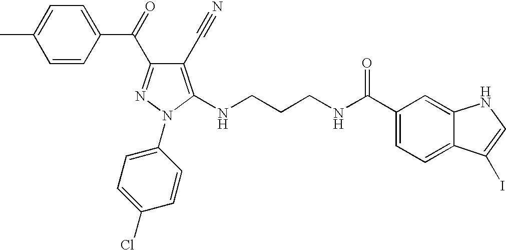 | C30H24ClIN6O2 | 663.3 |
| 228 | 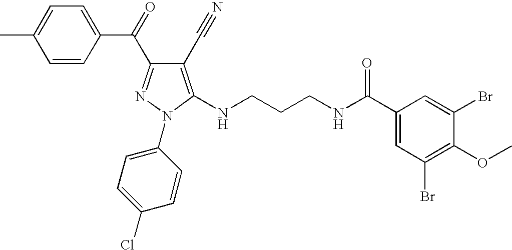 | C29H24Br2ClN5O3 | 686.1 |
| 229 | 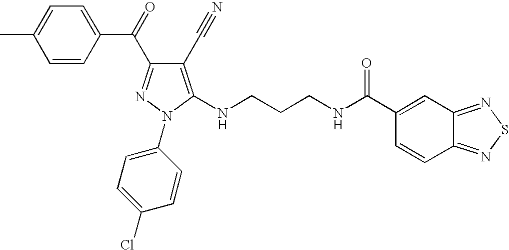 | C28H22ClN7O2S | 556 |
| 230 | 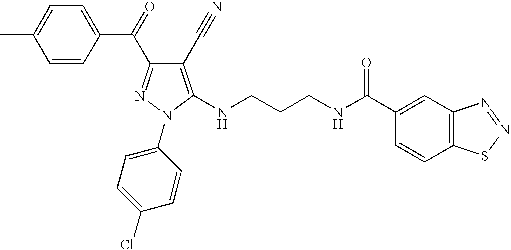 | C28H22ClN7O2S | 556 |
| 231 | 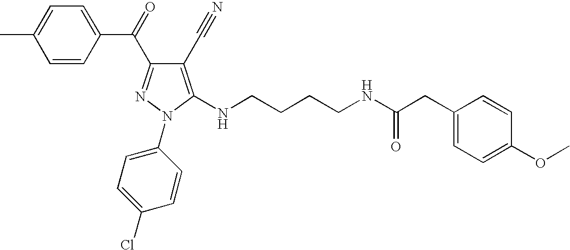 | C31H30ClN5O3 | 556 |
| 232 | 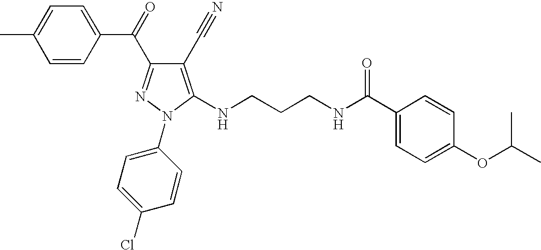 | C31H30ClN5O3 | 556 |

### Example 233

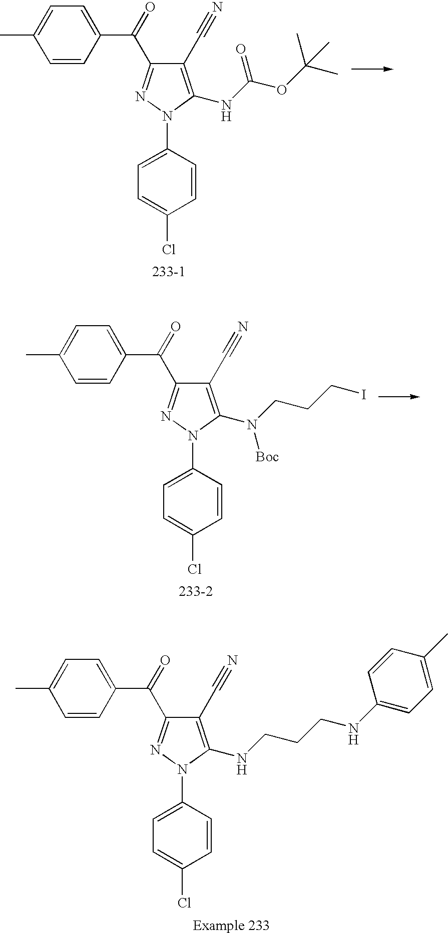

Step A: The amine of Intermediate I-1 was protected with a Boc group to give compound 233-1. Then compound 233-1 (6.99 g, 16.0 mmol) was added K₂CO₃ (4.42 g, 32.0 mmol) and DMF (40 mL), and the solution was stirred under ambient conditions for 1 h. The solution was quickly added to a solution of 1,3-diiodopropane (5.51 mL, 48.0 mmol) in DMF (40 mL), and the reaction was stirred under ambient conditions for 2 h. The crude product was isolated by aqueous workup and purified by column chromatograpy to yield compound 233-2. LCMS for C₂₆H₂₆ClIN₄O₃ [M+H⁺]: calc 605.1, found 605.6.
Step B: To compound 233-2 (50 mg, 0.083 mmol) was added potassium carbonate (27.6 mg, 0.200 mmol), p-toluidine (21.4 mg, 0.200 mmol), and DMF (1 mL). The reaction was stirred overnight under ambient conditions. The completed reaction was concentrated in vacuo. To the crude Boc-protected product was added 30% TFA/DCM (2 mL), and the reaction was stirred under ambient conditions for 1 hour. The reaction was concentrated in vacuo and purified by reverse-phase HPLC (YMC-Pack Pro C18, 100×20 mm, 5 μm, gradient 20-100 ACN/H₂O with 0.1% TFA) to yield Example 233. ¹H NMR (500 MHz, d6-DMSO) δ 8.03 (d, 2H), 7.64 (d, 2 H), 7.61 (d, 2H), 7.32 (d, 2H), 7.06 (broad, 2H), 6.98 (t, 1H), 6.82 (broad, 2H), 3.48 (m, 2H), 3.16 (t, 2H), 2.37 (s, 3H), 2.20 (s, 3H), 1.88 (m, 2H).

The compounds in Table 17 were prepared using the appropriate reagents following procedures similar to that described above for Example 233.

TABLE 17

| | | | Mass Found |
|---|---|---|---|
| Example | Structure | Parent Formula | [M + H] |
| 234 | 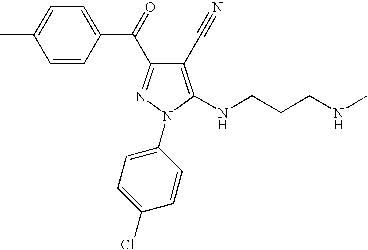 | C22H22ClN5O | 408.3 |
| 235 | 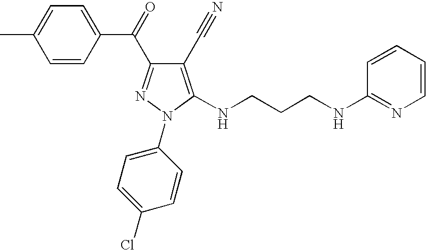 | C26H23ClN6O | 471.3 |
| 236 | 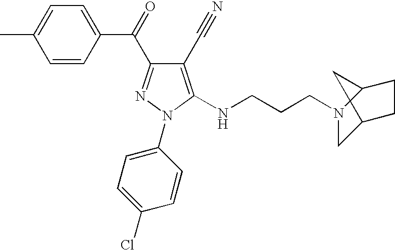 | C27H28ClN5O | 474.7 |
| 237 | 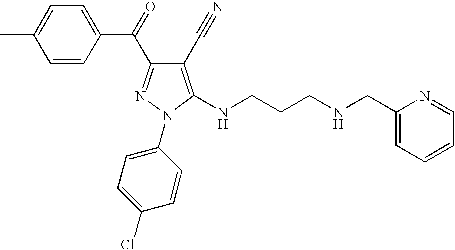 | C27H25ClN6O | 485.4 |
| 238 | 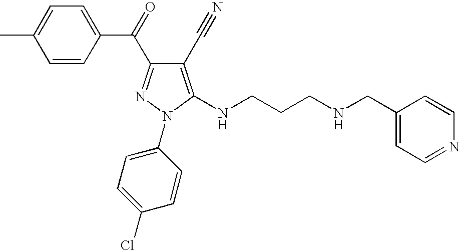 | C27H25ClN6O | 485.4 |
| 239 | 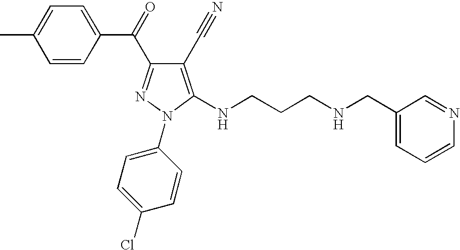 | C27H25ClN6O | 485.9 |
| 240 | 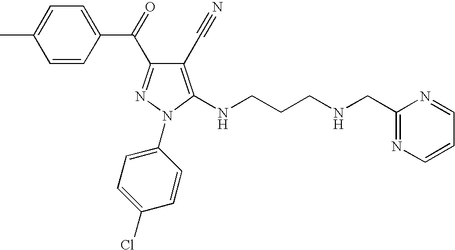 | C26H24ClN7O | 486.7 |
| 241 | 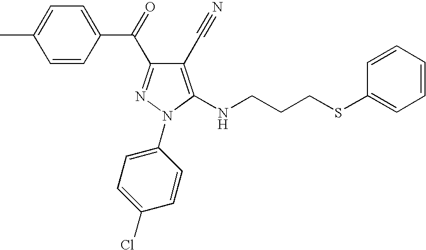 | C27H23ClN4OS | 487.4 |
| 242 | 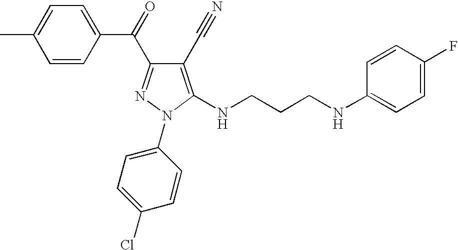 | C27H23ClFN5O | 488.3 |
| 243 | 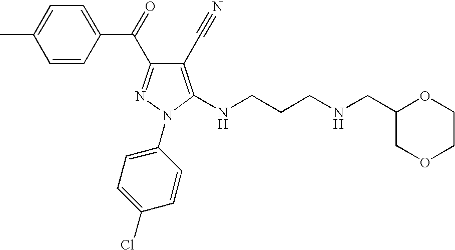 | C26H28ClN5O3 | 494.3 |
| 244 | 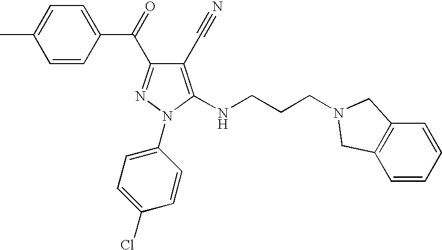 | C29H26ClN5O | 496.9 |
| 245 | 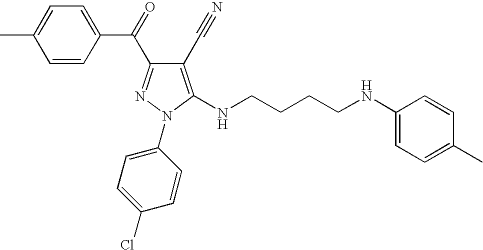 | C29H28ClN5O | 498.3 |
| 246 | 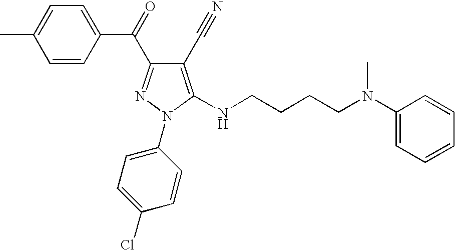 | C29H28ClN5O | 498.3 |
| 247 | 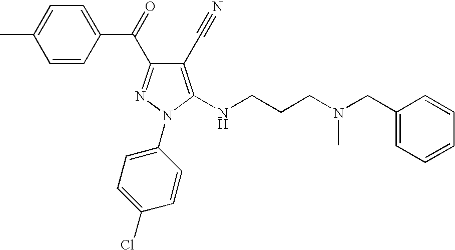 | C29H28ClN5O | 498.7 |
| 248 | 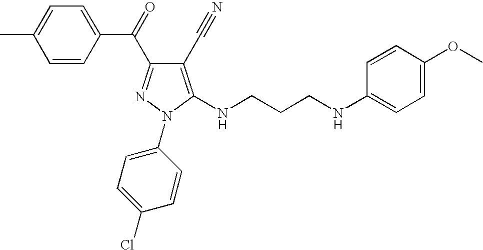 | C28H26ClN5O2 | 500.3 |
| 249 | 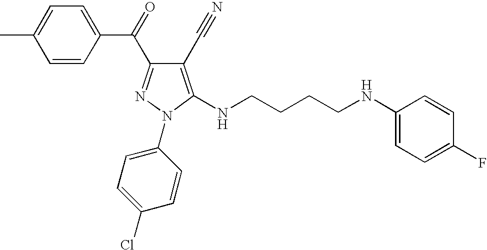 | C28H25ClFN5O | 502.3 |
| 250 | 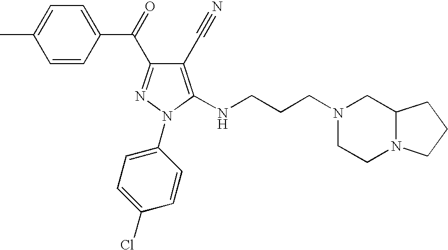 | C28H31ClN6O | 503.4 |
| 251 | 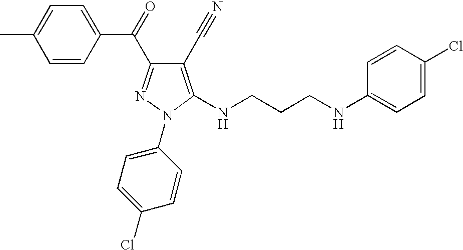 | C27H23Cl2N5O | 504.2 |
| 252 | 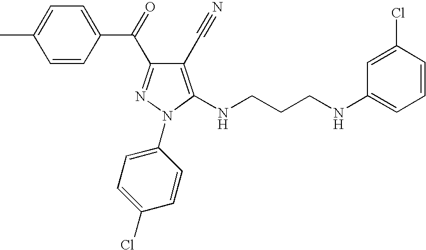 | C27H23Cl2N5O | 504.7 |
| 253 | 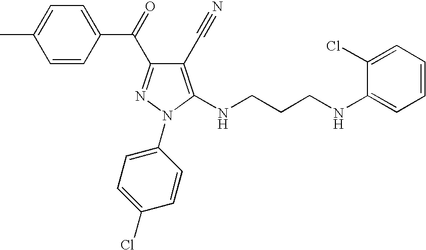 | C27H23Cl2N5O | 504.7 |
| 254 | 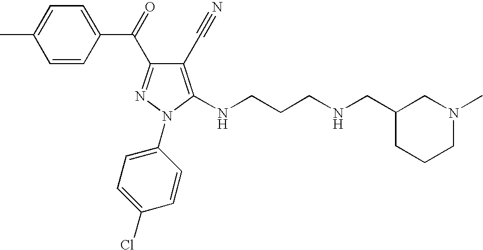 | C28H33ClN6O | 505.3 |
| 255 | 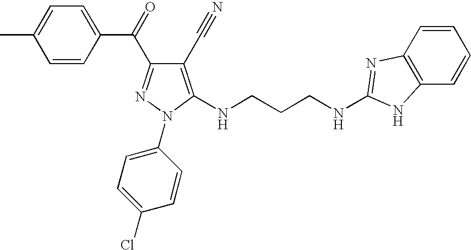 | C28H24ClN7O | 510.3 |
| 256 | 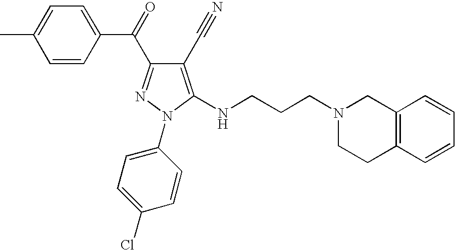 | C30H28ClN5O | 510.3 |
| 257 | 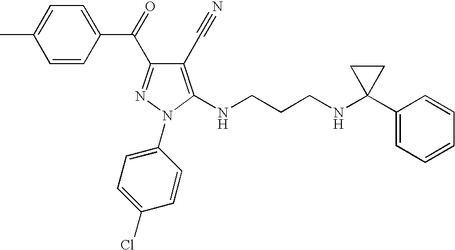 | C30H28ClN5O | 510.3 |
| 258 | 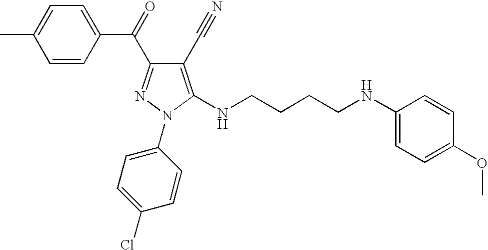 | C29H28ClN5O2 | 514.3 |
| 259 | 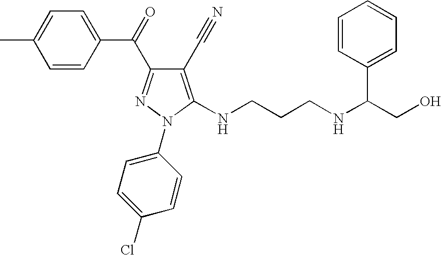 | C29H28ClN5O2 | 514.5 |
| 260 | 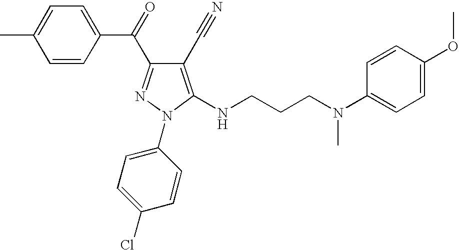 | C29H28ClN5O2 | 514.7 |
| 261 | 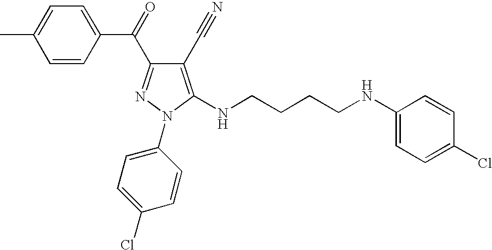 | C28H25Cl2N5O | 518.3 |
| 262 | 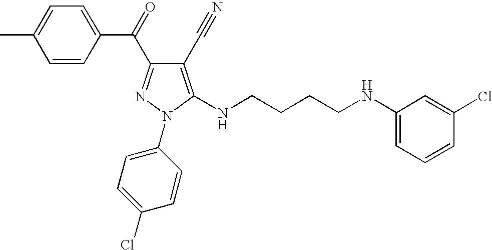 | C28H25Cl2N5O | 518.3 |
| 263 | 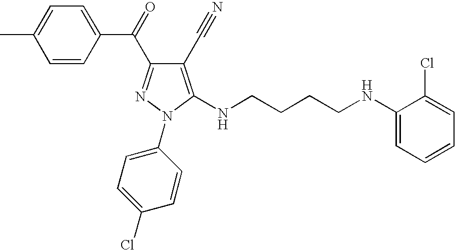 | C28H25Cl2N5O | 518.3 |
| 264 | 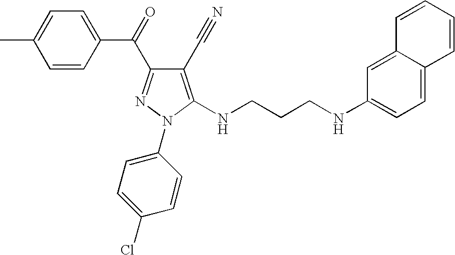 | C31H26ClN5O | 520.7 |
| 265 | 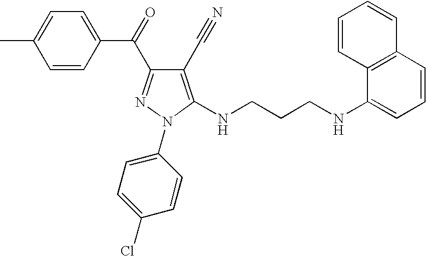 | C31H26ClN5O | 520.7 |
| 266 | 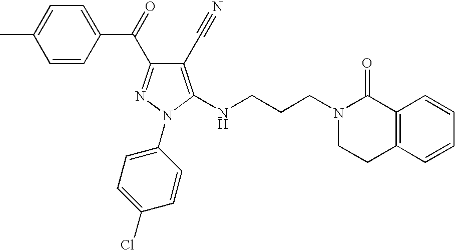 | C30H26ClN5O2 | 524.3 |
| 267 | 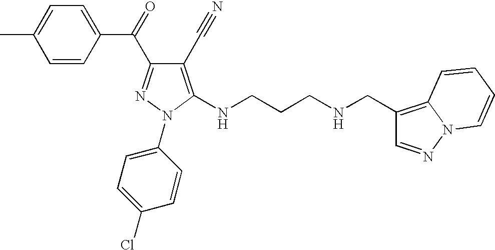 | C29H26ClN7O | 524.5 |
| 268 | 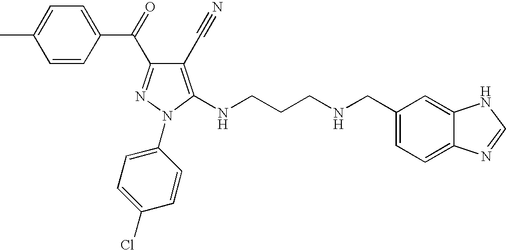 | C29H26ClN7O | 524.5 |
| 269 | 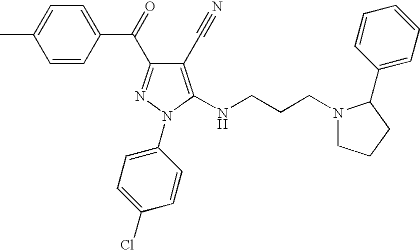 | C31H30ClN5O | 524.9 |
| 270 | 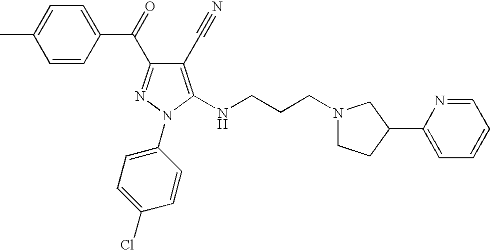 | C30H29ClN6O | 525.5 |
| 271 | 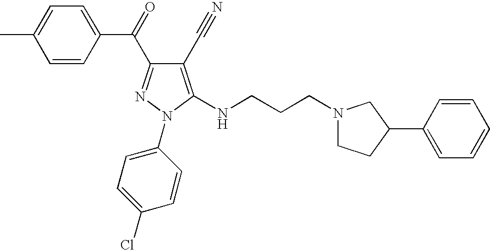 | C31H30ClN5O | 525.8 |
| 272 | 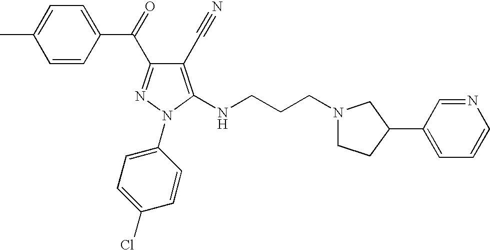 | C30H29ClN6O | 526.0 |
| 273 | 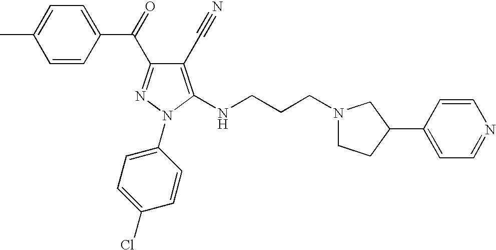 | C30H29ClN6O | 526.0 |
| 274 | 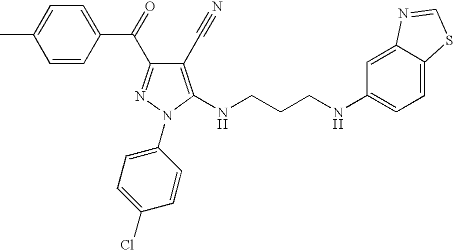 | C28H23ClN6OS | 527.2 |
| 275 | 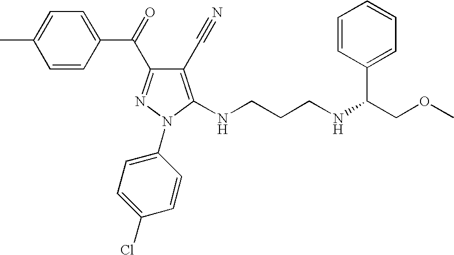 | C30H30ClN5O2 | 528.5 |
| 276 | 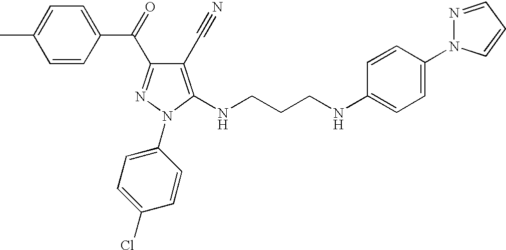 | C30H26ClN7O | 536.7 |
| 277 | 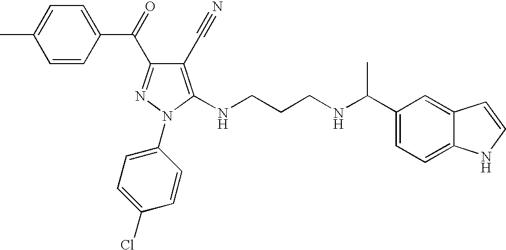 | C31H29ClN6O | 536.9 |
| 278 | 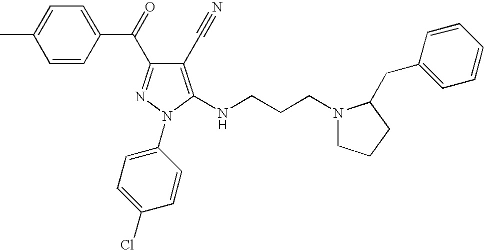 | C32H32ClN5O | 538.5 |
| 279 | 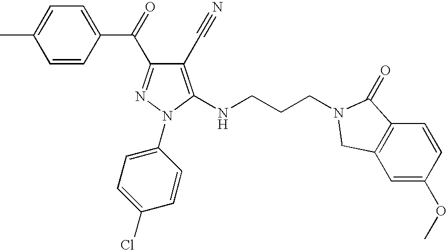 | C30H26ClN5O3 | 540.3 |
| 280 | 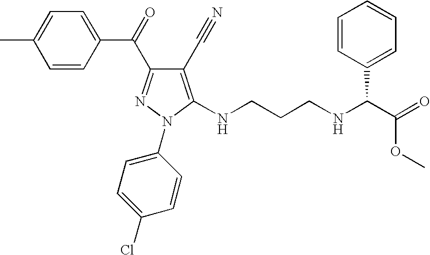 | C30H28ClN5O3 | 542.5 |
| 281 | 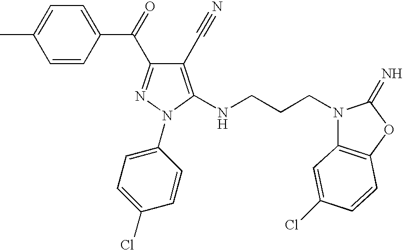 | C28H22Cl2N6O2 | 545.2 |
| 282 | 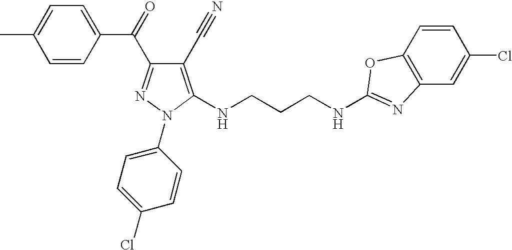 | C28H22Cl2N6O2 | 545.2 |
| 283 | 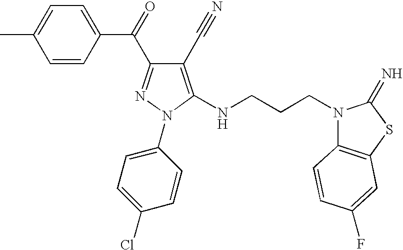 | C28H22ClFN6OS | 545.3 |
| 284 | 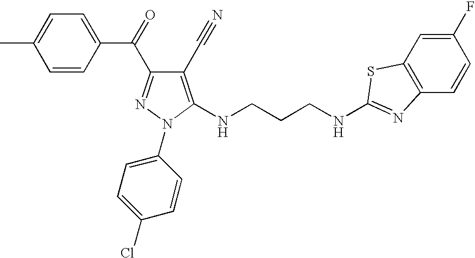 | C28H22ClFN6OS | 545.3 |
| 285 | 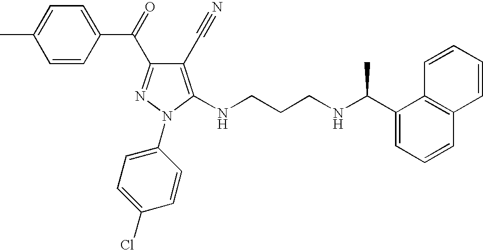 | C33H30ClN5O | 548.6 |
| 286 | 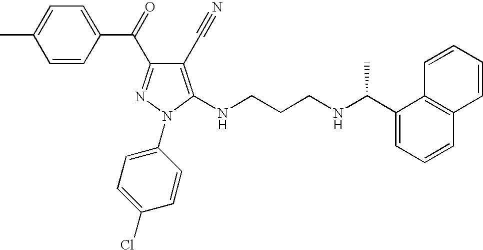 | C33H30ClN5O | 548.6 |
| 287 | 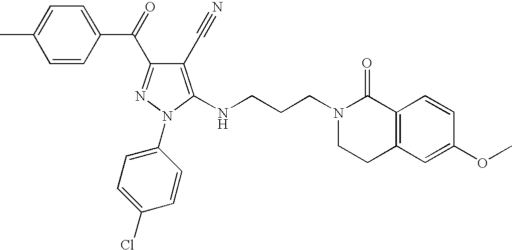 | C31H28ClN5O3 | 554.3 |
| 288 | 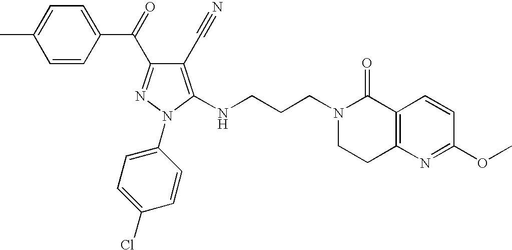 | C30H27ClN6O3 | 555.3 |
| 289 | 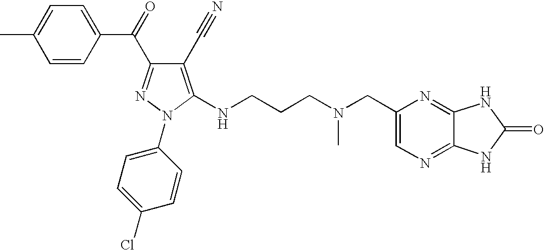 | C28H26ClN9O2 | 556.5 |
| 290 | 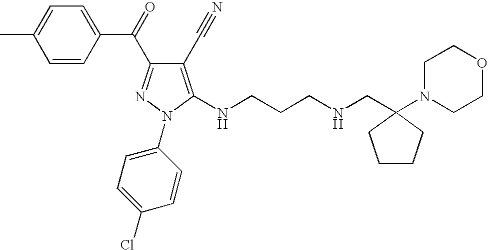 | C31H37ClN6O2 | 561.8 |
| 291 | 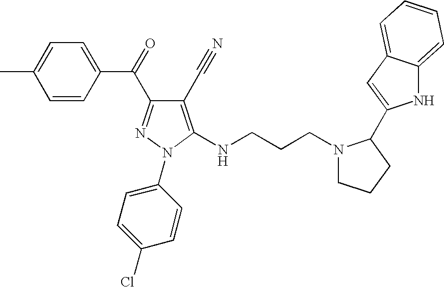 | C33H31ClN6O | 563.6 |
| 292 | 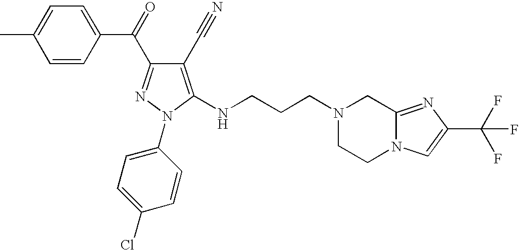 | C28H25ClF3N7O | 568.3 |
| 293 | 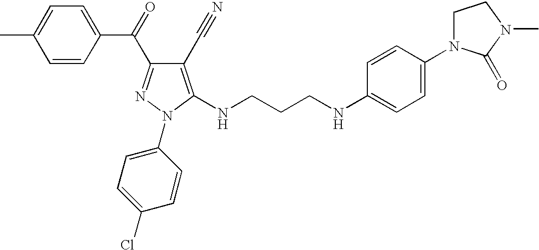 | C31H30ClN7O2 | 568.8 |
| 294 | 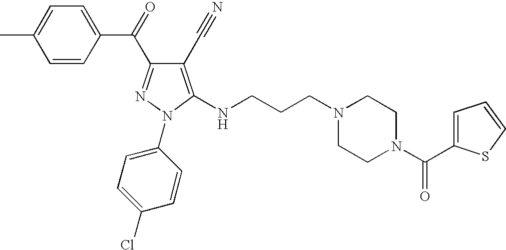 | C30H29ClN6O2S | 573.3 |
| 295 | 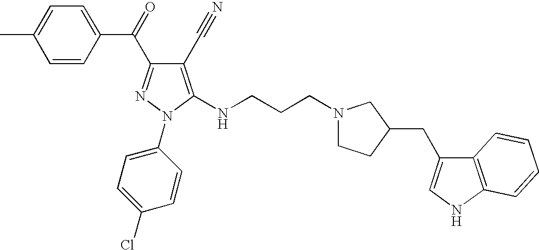 | C34H33ClN6O | 578.0 |
| 296 | 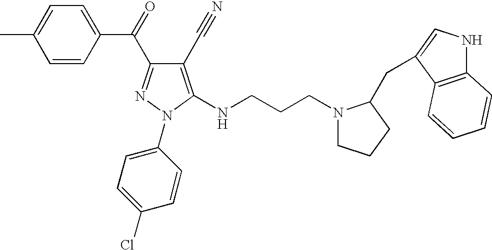 | C34H33ClN6O | 578.0 |

### Example 297

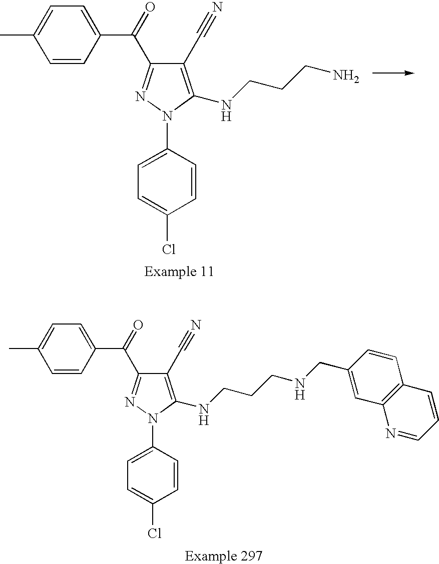

To Example 11 (34.0 mg, 0.087 mmol) was added quinoline-7-carbaldehyde (13.7 mg, 0.087 mmol) and THF (2 mL). The reaction was stirred overnight under ambient conditions for 4 h to allow imine formation. To the reaction was added sodium triacetoxyborohydride (74 mg, 0.35 mmol), and the reaction was stirred overnight under ambient conditions. The completed reaction was purified by reverse-phase HPLC (YMC-Pack Pro C18, 100×20 mm, 5 μm, gradient 20-100 ACN/H₂O with 0.1% TFA) to yield Example 297. ¹H NMR (500 MHz, d6-DMSO) δ 9.0-8.8 (broad, 3H), 8.42 (d, 1H), 8.19 (s, 1H), 8.08 (d, 1H), 8.04 (d, 2H), 7.74-7.56 (m, 6H), 7.33 (d, 2H), 7.00 (t, 1H), 4.43 (m, 2H), 3.48 (m, 2H), 3.04 (broad, 2H), 2.38 (s, 3H), 2.00 (m, 2H).

The compounds in Table 18 were prepared using the appropriate reagents following procedures similar to that described above for Example 297.

TABLE 18

| | | | Mass Found |
|---|---|---|---|
| Example | Structure | Parent Formula | [M + H] |
| 298 | 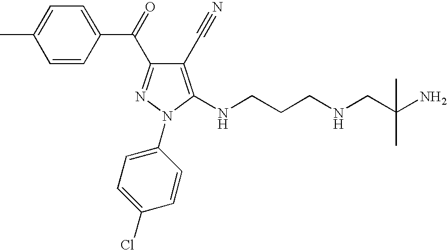 | C25H29ClN6O | 465.9 |
| 299 | 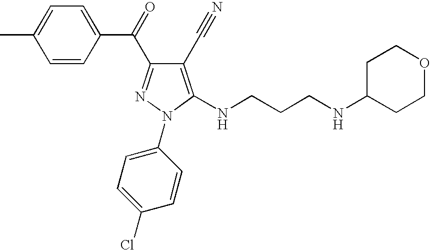 | C26H28ClN5O2 | 478.7 |
| 300 | 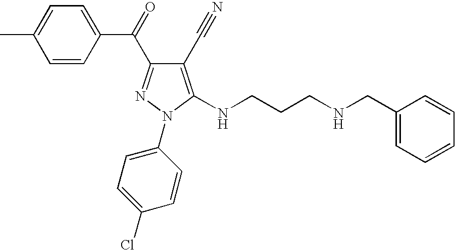 | C28H26ClN5O | 484.1 |
| 301 | 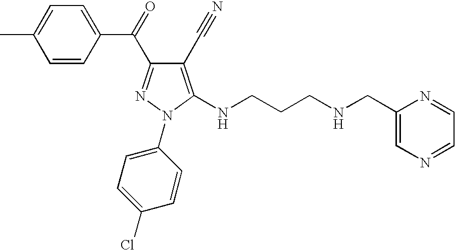 | C26H24ClN7O | 486.5 |
| 302 | 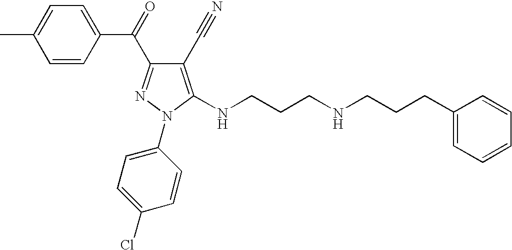 | C30H30ClN5O | 512.6 |
| 303 | 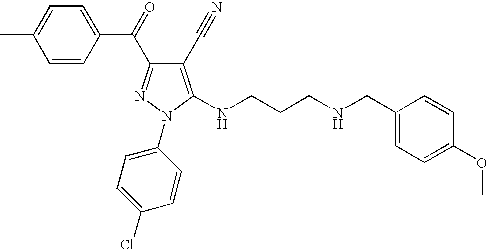 | C29H28ClN5O2 | 514.2 |
| 304 | 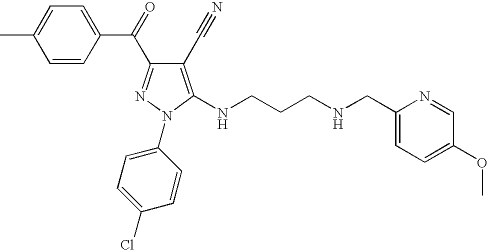 | C28H27ClN6O2 | 515.6 |
| 305 | 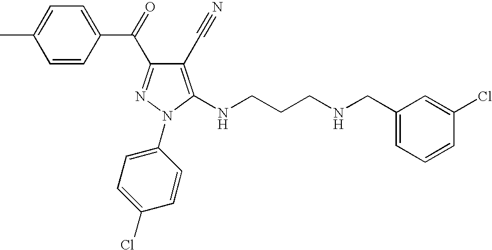 | C28H25Cl2N5O | 518.2 |
| 306 | 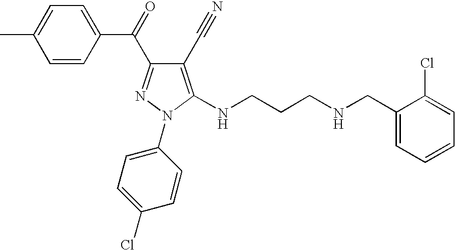 | C28H25Cl2N5O | 518.5 |
| 307 | 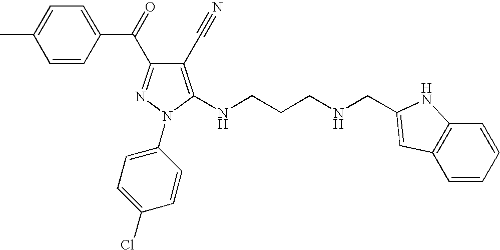 | C30H27ClN6O | 523.5 |
| 308 | 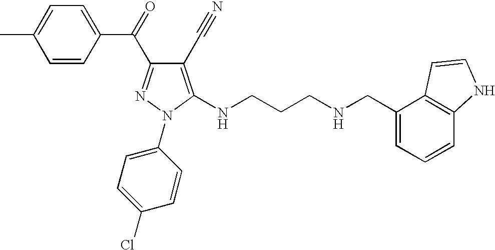 | C30H27ClN6O | 523.5 |
| 309 | 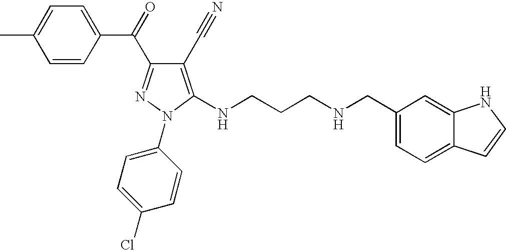 | C30H27ClN6O | 523.4 |
| 310 | 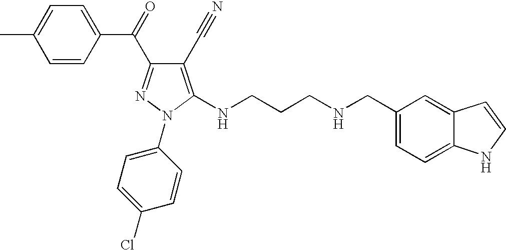 | C30H27ClN6O | 523.4 |
| 311 | 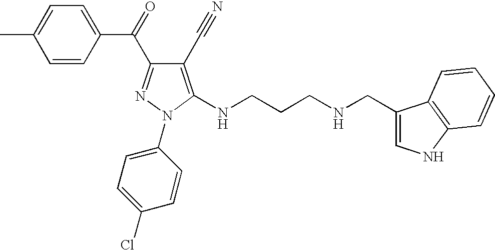 | C30H27ClN6O | 523.4 |
| 312 | 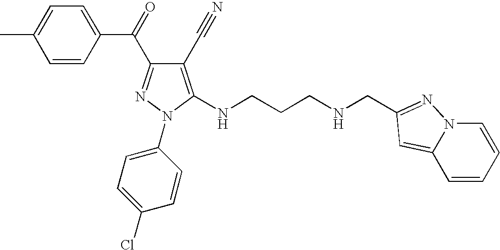 | C29H26ClN7O | 524.5 |
| 313 | 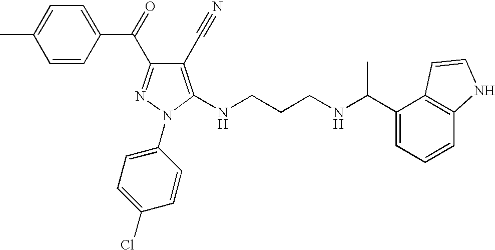 | C31H29ClN6O | 537.6 |
| 314 | 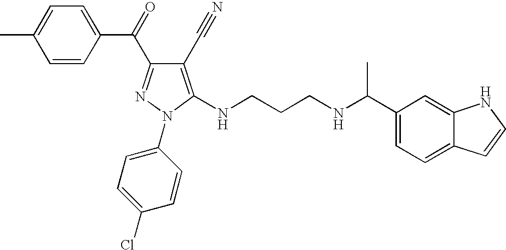 | C31H29ClN6O | 537.9 |
| 315 | 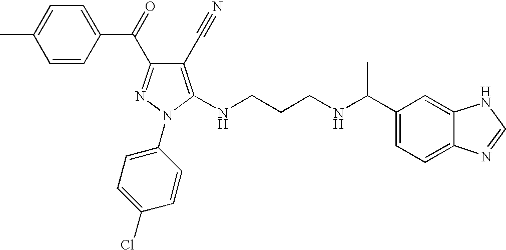 | C30H28ClN7O | 538.6 |
| 316 | 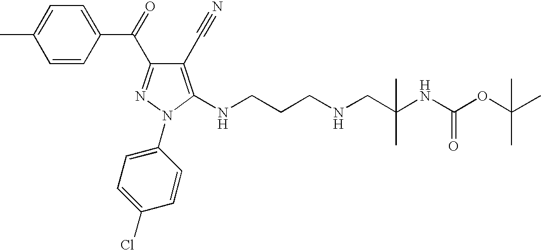 | C30H37ClN6O3 | 565.6 |

### Example 317

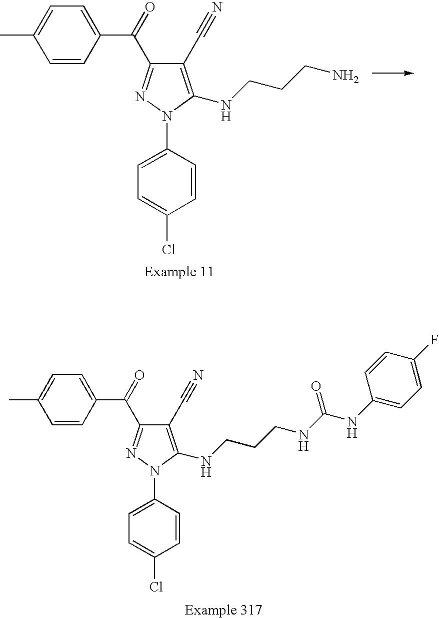

To Example 11 (TFA salt, 25.0 mg, 0.049 mmol) in DCM (1 mL) was added p-fluorophenylisocyanate (5.6 μL, 0.049 mmol) and triethylamine (6.8 μL). The reaction was stirred overnight under ambient conditions. The product was isolated by vacuum filtration and cold DCM washes to yield Example 317. LC-MS for C₂₈H₂₄ClFN₆O₂ [M+H]: calculated 531.2, found 531.2.

The compounds in Table 19 were prepared using the appropriate reagents following procedures similar to that described above for Example 317.

TABLE 19

| | | | Mass Found |
|---|---|---|---|
| Example | Structure | Parent Formula | [M + H] |
| 318 | 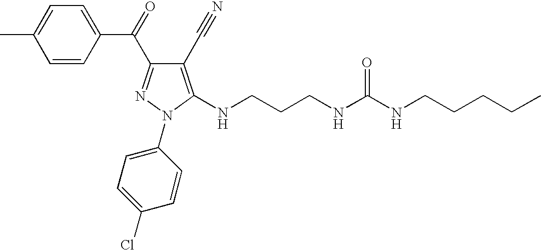 | C27H31ClN6O2 | 507.6 |
| 319 | 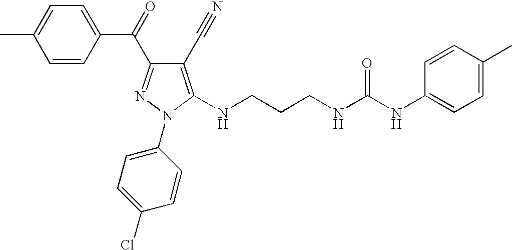 | C29H27ClN6O2 | 527.2 |
| 320 | 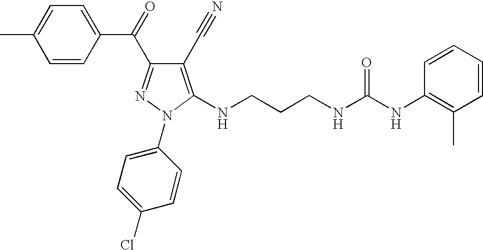 | C29H27ClN6O2 | 527.5 |
| 321 | 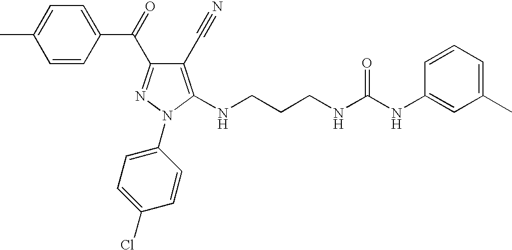 | C29H27ClN6O2 | 527.6 |
| 322 | 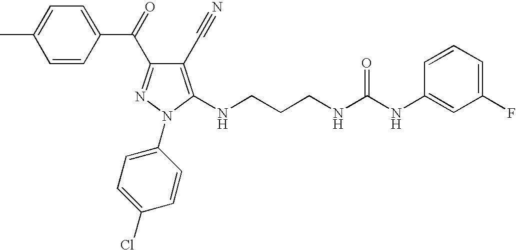 | C28H24ClFN6O2 | 531.4 |
| 323 | 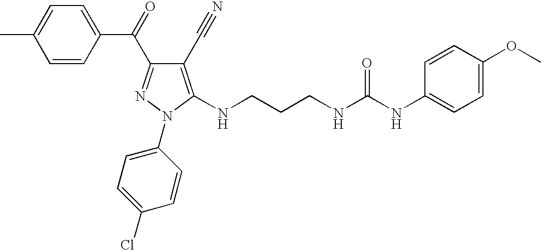 | C29H27ClN6O3 | 543.5 |
| 324 | 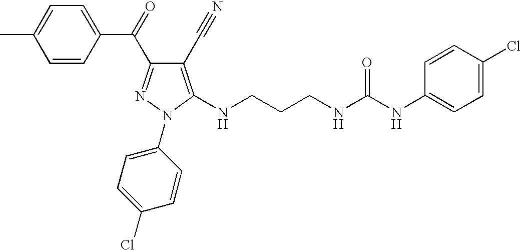 | C28H24Cl2N6O2 | 547.2 |
| 325 | 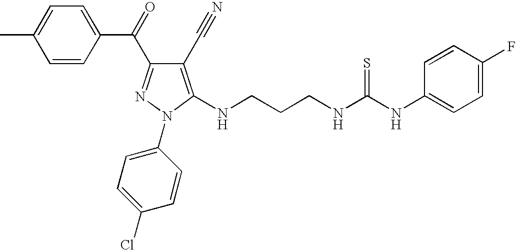 | C28H24ClFN6OS | 547.7 |
| 326 | 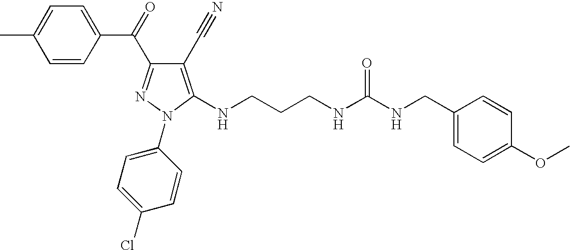 | C30H29ClN6O3 | 557.6 |
| 327 | 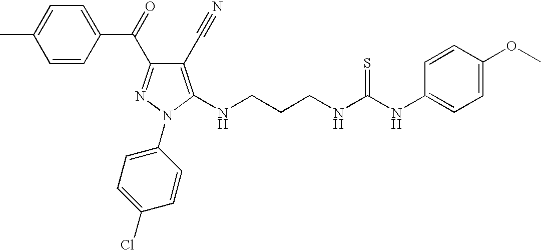 | C29H27ClN6O2S | 559.2 |
| 328 | 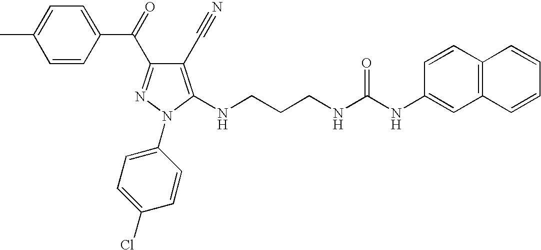 | C32H27ClN6O2 | 563.3 |
| 329 | 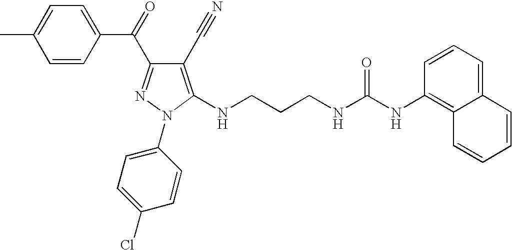 | C32H27ClN6O2 | 563.5 |
| 330 | 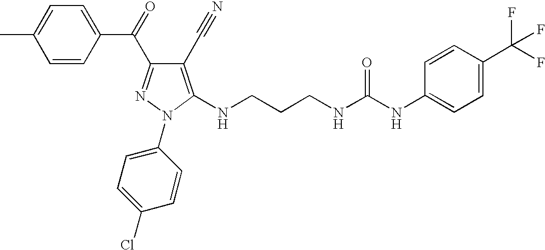 | C29H24ClF3N6O2 | 581.5 |

### Example 331

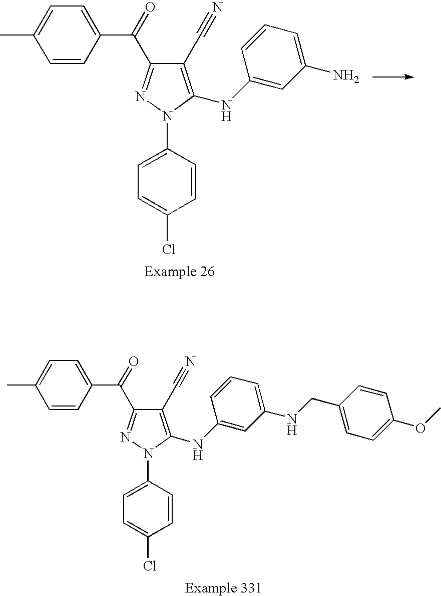

To Example 26 (6.0 mg, 0.014 mmol) in DMF (1 mL) and acetonitrile (1 mL) was added diisopropylethyl amine (7.3 μL, 0.042 mmol) and p-methoxybenzylchloride (2.2 mg, 0.014 mmol). The reaction was stirred under nitrogen at ambient temperature for 2 days. The completed reaction was purified by reverse-phase HPLC (YMC-Pack Pro C18, 100×20 mm, 5 μm, gradient 20-100 ACN/H₂O with 0.1% TFA) to yield Example 331. LC-MS for C₃₂H₂₆ClN₅O₂ [M+H]: calculated 549.0, found 548.9.

The compounds in Table 20 were prepared using the appropriate reagents following procedures similar to that described above for Example 331.

TABLE 20

| | | | Mass Found |
|---|---|---|---|
| Example | Structure | Parent Formula | [M + H] |
| 332 | 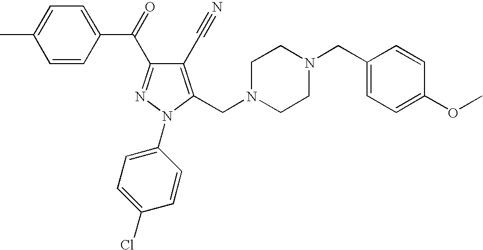 | C31H30ClN5O2 | 540.6 |
| 333 | 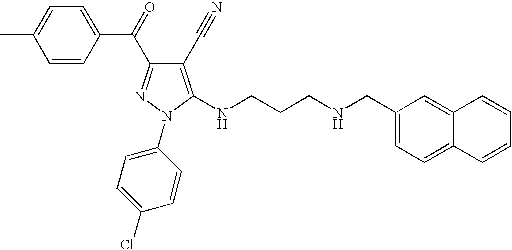 | C32H28ClN5O | 534.6 |

### Example 334

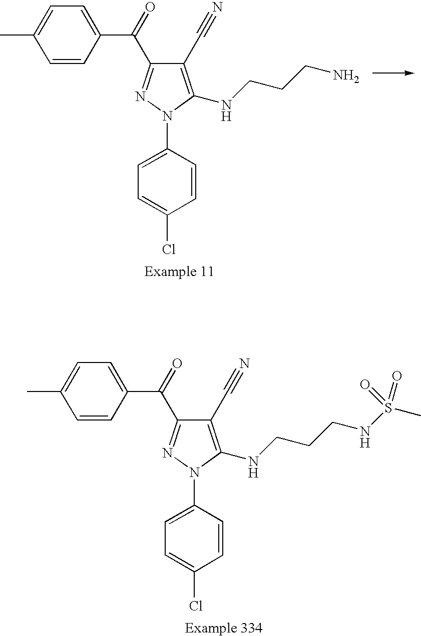

To a solution of Example 11 (67 mg, 0.17 mmol) in pyridine (1 mL) was added methanesulfonic anhydride (30 mg, 0.173 mmol). The reaction was stirred under nitrogen at ambient temperature for 1 day. The completed reaction was purified by reverse-phase HPLC (YMC-Pack Pro C18, 100×20 mm, 5 μm, gradient 20-100 ACN/H₂O with 0.1% TFA) to yield Example 334. LC-MS for C₂₂H₂₂ClN₅O₃S [M+H]: calculated 472.1, found 472.1.

The compounds in Table 21 were prepared using the appropriate reagents following procedures similar to that described above for Example 334.

TABLE 21

| | | | Mass Found |
|---|---|---|---|
| Example | Structure | Parent Formula | [M + H] |
| 335 | 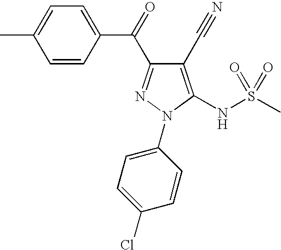 | C19H15ClN4O3S | 415.1 |
| 336 | 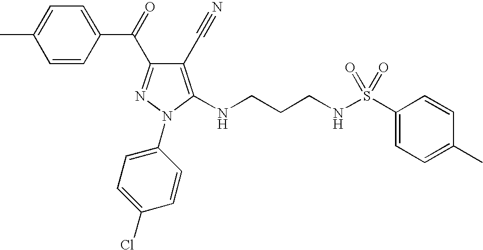 | C28H26ClN5O3S | 548.2 |

### Example 337

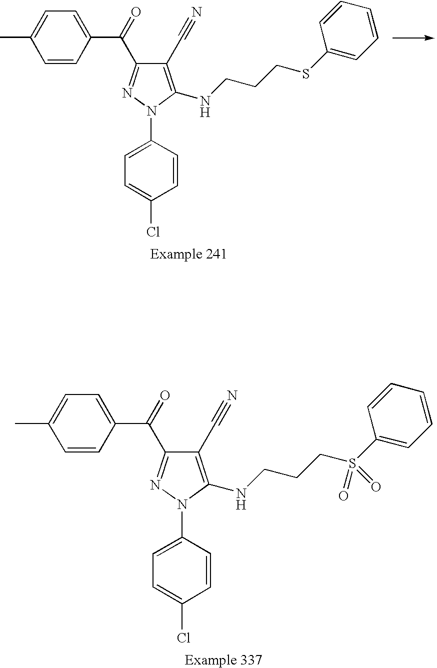

To a solution of Example 241 (68 mg, 0.14 mmol) in DCM (2 mL) was added m-CPBA (50 mg, 0.28 mmol). The reaction was stirred under nitrogen at ambient temperature for 1 h. The completed reaction was diluted with DCM and quenched with excess Ca(OH)₂ and stirring for 10 min. After vacuum filtration, the filtrate was concentrated in vacuo and purified by reverse-phase HPLC (YMC-Pack Pro C18, 100×20 mm, 5 μm, gradient 20-100 ACN/H₂O with 0.1% TFA) to yield Example 337.

### Example 338

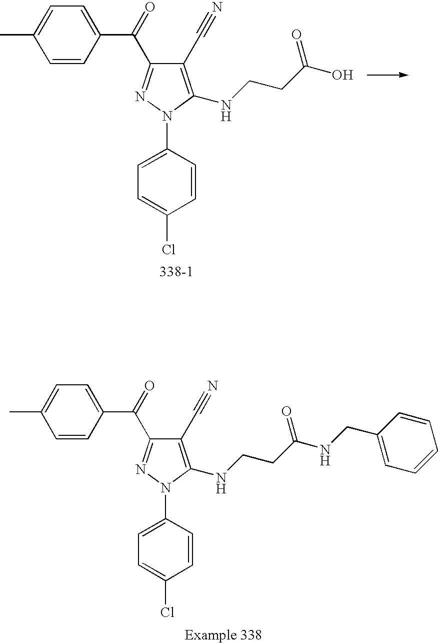

Compound 338-1 may be prepared according to the procedure for Example 28, using the appropriate reagents. To a solution of compound 338-1 (14 mg, 0.034 mmol) in DCM (1 mL) was added EDAC.HCl coupling reagent (20 mg, 0.104 mmol), DMAP (cat.), and benzyl amine (20 uL, 0.183 mmol). The reaction was stirred under nitrogen at ambient temperature for 2 h. The crude product was isolated by aqueous workup and purified by reverse-phase HPLC (YMC-Pack Pro C18, 100×20 mm, 5 μm, gradient 20-100 ACN/H₂O with 0.1% TFA) to yield Example 338. LC-MS for C₂₈H₂₄ClN₅O₂ [M+H]: calculated 498.1, found 498.1.

The compound in Table 22 was prepared using the appropriate reagents following procedures similar to that described above for Example 338.

TABLE 22

| | | | Mass Found |
|---|---|---|---|
| Example | Structure | Parent Formula | [M + H] |
| 339 | 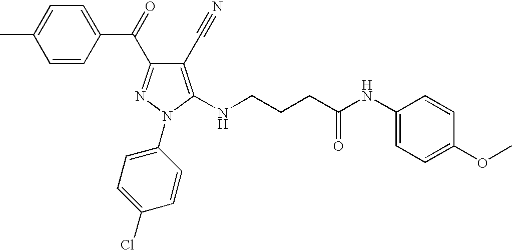 | C29H26ClN5O3 | 528.3 |

### Example 340

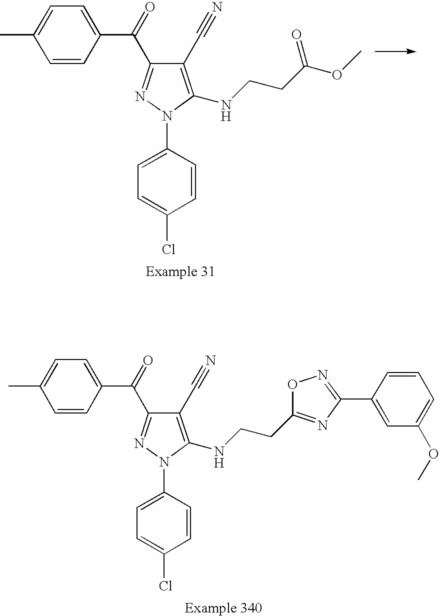

To a solution of 3-methoxybenzamide oxime (70 mg, 0.422 mmol) in THF (2 mL) was added sodium hydride (60% in oil, 17 mg, 0.425 mmol). The reaction was stirred under nitrogen at ambient temperature for 10 min. To the reaction was added Example 31 (60 mg, 0.141 mmol). The reaction was stirred under nitrogen at 80° C. for 2 h. The completed reaction was purified by reverse-phase HPLC (YMC-Pack Pro C18, 100×20 mm, 5 μm, gradient 20-100 ACN/H₂O with 0.1% TFA) to yield Example 340. ¹H NMR (500 MHz, d6-DMSO) δ 7.98 (d, 2H), 7.60 (m, 4H), 7.54 (d, 1H), 7.46 (m, 2H), 7.30 (d, 2H), 7.15 (d, 1H), 7.07 (t, 1H), 3.93 (m, 2H), 3.79 (s, 3H), 3.35 (m, 2H), 2.37 (s, 3H).

The compounds in Table 23 were prepared using the appropriate reagents following procedures similar to that described above for Example 340.

TABLE 23

| | | | Mass Found |
|---|---|---|---|
| Example | Structure | Parent Formula | [M + H] |
| 341 | 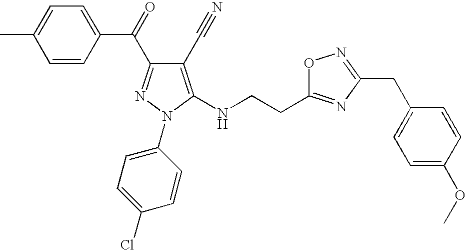 | C30H25ClN6O3 | 553.5 |
| 342 | 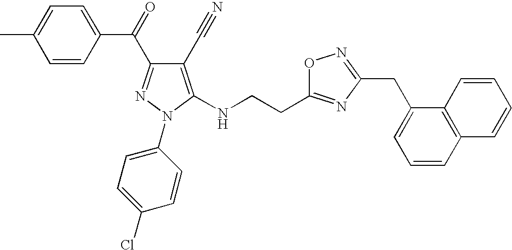 | C33H25ClN6O2 | 573.2 |

### Example 343

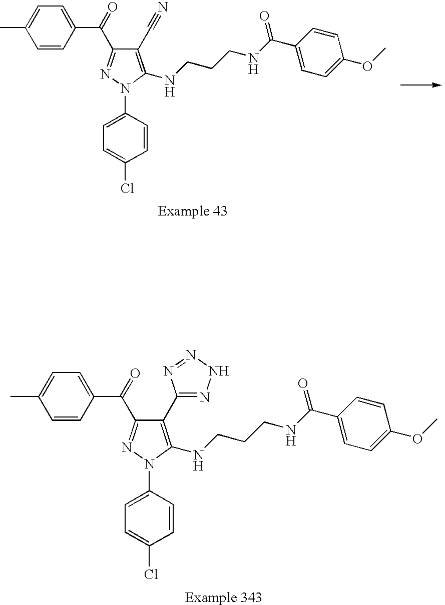

To Example 43 (69 mg, 0.13 mmol) was added zinc (II) chloride (44.5 mg, 0.33 mmol), sodium azide (42 mg, 0.63 mmol), and DMF (1 mL). The reaction was refluxed under nitrogen for 24 h. The completed reaction was quenched with 0.1 M aqueous HCl. The solid was collected by vacuum filtration and purified by reverse-phase HPLC (YMC-Pack Pro C18, 100×20 mm, 5 μm, gradient 20-100 ACN/H₂O with 0.1% TFA) and prep TLC to yield Example 343. ¹H NMR (500 MHz, d6-DMSO) δ 8.17 (t, 1H), 8.02 (d, 2H), 7.72 (m, 4H), 7.60 (d, 2H), 7.33 (d, 2H), 6.96 (d, 2H), 6.30 (t, 1H), 3.79 (s, 3H), 3.05 (m, 2H), 2.81 (m, 2H), 2.38 (s, 3H), 1.47 (m, 2H).

### Biological Assays

### A. Binding Assay

The membrane binding assay is used to identify competitive inhibitors of ¹²⁵I-ghrelin binding to cloned human, mouse, and/or rat ghrelin receptor expressed in COS-cells.

The ghrelin receptor is transiently expressed in COS cells transfected by electroporation. COS cells are grown in medium of the composition: 1 L Dulbecco's Modified Eagles Medium (DMEM) with 4.5 g L-glucose, 25 mM Hepes, without sodium pyruvate; 100 ml fetal bovine serum; 10 mL 10,000 unit/mL penicillin & 10,000 μg/mL streptomycin; and 10 ml 200 mM L-glutamine (all cell media reagents are from Invitrogen-Gibco). The cells are grown in T-175 flasks at 37° C. with CO₂ and humidity control until the desired cell density and cell number is obtained.

Prior to electroporation the cells are detached with 0.5% trypsin/EDTA. The cells are collected in growth media, harvested by centrifugation, and re-suspended in phosphate buffered saline (PBS) without calcium or magnesium. The cells are harvested and re-suspended in PBS a second time, diluted to a density of 1.2×10⁷ cells/ml, and 0.85 ml combined with 20 μg ghrelin receptor plasmid DNA and electroporated. The transfected cells are transferred to fresh growth media in T-175 flasks and incubated at 37° C. with CO₂ for 3 days before harvesting for membrane preparation.

The medium is poured off and 10 mL/flask of enzyme-free dissociation media (Specialty Media Inc.) is added. The cells are incubated at 37° C. for 10 min or until cells sloughed off when flask is banged against hand. The cells are harvested into 200 mL centrifuge tubes and spun at 1000 rpm, 4° C., for 10 min. The supernatant is discarded and the cells are resuspended in 5 mL/monolayer membrane preparation buffer having the composition: 10 mM Tris pH 7.2-7.4; 4 μg/mL Leupeptin (Sigma); 10 μM Phosphoramidon (Boehringer Mannheim); 40 μg/mL Bacitracin (Sigma); 5 μg/mL Aprotinin (Sigma); 10 mM Pefabloc (Boehringer Mannheim). The cells are homogenized with motor-driven dounce (Talboy setting 40), using 10 strokes and the homogenate centrifuged at 6,000 rpm, 4° C., for 15 min.

The pellets are re-suspended in 0.2 mL/monolayer membrane prep buffer and aliquots are placed in tubes (500-1000 μL/tube) and quick frozen in liquid nitrogen and then stored at −80° C.

Test compounds are diluted in dimethylsulfoxide (DMSO) (10⁻⁵ to 10⁻¹⁰ M) (5 μL) are added to 145 μL of membrane binding buffer containing ghrelin receptor membrane protein (5-40 μg). The membrane binding buffer had the composition: 25 mM Tris pH 7.4; 10 mM MgCl₂; 2.5 mM EDTA; 0.1% BSA; 5 μg/mL Leupeptin (SIGMA); 40 μg/mL Bacitracin (SIGMA); 5 μg/1 mL Aprotinin (SIGMA); and 10 mM Pefabloc (Boehringer Mannheim). Fifty μL of radiolabeled ghrelin [¹²⁵I-ghrelin (Perkin-Elmer) diluted in binding buffer to 250 CPM/uL] is added and the resulting mixture is vortexed briefly and incubated for 90-120 min at room temp while shaking.

The mixture is filtered on a Packard Microplate 196 filter apparatus using Millipore Multiscreen GF/C 96-well filter plates pretreated with 0.5% polyethyleneimine (Sigma). The filter is washed 3×2 mL with cold wash buffer having the composition: 50 mM Tris-HCl pH 7.4; 10 mM MgCl₂; 2.5 mM EDTA. The filter is dried, and the bottom sealed and 50 μL of Packard Microscint-20 is added to each well. The top is sealed and the radioactivity quantitated in a Packard Topcount Microplate Scintillation counter.

### B. Functional Assay

Functional cell based assays are developed to discriminate ghrelin receptor agonists, inverse agonists, and antagonists.

CHO/NFAT/beta-lactamase cells stably expressing the human ghrelin receptor are maintained in Iscove's media supplemented with 10% FBS, 1× glutamine, 1× pen/strep, 0.1 mg/ml zeocin, and 1.25 mg/ml G418). Cells are detached from T-175 flasks with 0.5% trypsin, plated at 6000 cells/well in 0.2 ml in a 96-well plate (black clear bottom plate, Corning #3614), and incubated at 37° C. with CO₂ for 2 days prior to assay.

Test compounds are diluted in dimethylsulfoxide (DMSO) (10⁻⁵ to 10⁻¹⁰ M) and added to the cell plate (0.25% DMSO final). Plates are incubated at 37° C. with CO₂ for 3 hours and the media replaced with 100 ul of CCF4-lactamase substrate loading media (Invitrogen). The cells are loaded during a 1 hour incubation at room temperature in the dark and the background subtracted fluorescence emission ratio (460/530 nm) is measured on a Molecular Devices Analyst-HT microplate reader. Dose-response curves are plotted using GraphPad Prism software. Inositol phosphate accumulation can be measured in cells (CHO or HEK) expressing the ghrelin receptor. For example, a stable ghrelin receptor HEK cell line is maintained in DMEM-high glucose, 10% FBS, 1× pen/strep/glutamine, 25 mM HEPES, 0.5 milligrams/ml G418, and 0.2 milligrams/ml hygromycin, detached with 0.5% trypsin, and plated in poly-lysine coated plates. The following day, the media is replaced with 150 microliters of 3H-inositol labeling media (inositol-free DMEM (DMEM with 4500 mg/L glucose, without L-glutamine & i-inositol; ICN #1642954) supplemented with 10% FBS, 1× pen/strep/glutamine, 25 mM HEPES, and the appropriate selection antibiotics as described above, to which is added 3H-myo-inositol (NEN #NET114A, 1 mCi/ml, 25 Ci/mmol) diluted 1:150 in loading medium (final specific radioactivity of 1 uCi/150 microliter). The following day the cell monolayer is confluent and 5 ul 300 mM LiCl is added to all wells (10 mM final) and the plates incubated 20 minutes at 37° C. Test compounds are diluted in dimethylsulfoxide (DMSO) (10⁻⁵ to 10⁻¹⁰ M), added to the cell plate (0.5% DMSO final), the plate incubated at 37 C for 1 hour, and the media aspirated. The assay is terminated by addition of 60 ul 10 mM formic acid and the cells are lysed for 60 minutes at room temperature. A sample of lysate (10-30 uL) is transferred to a 96-well white clear-bottom Optiplates containing 1 mg/well RNA binding YSi SPA-beads (Amersham RPNQ0013). The plates are shaken for 2 hr at room temperature and counted on a Wallac Microbeta Trilux. Dose-response curves are plotted using GraphPad Prism software. Inverse agonists are identified by dose-dependent inhibition of the basal level of inositol phosphate accumulation.

Antagonist assay: Antagonist activity is defined as the ability of a compound to block a functional response to ghrelin. A solution of test compound is added to the cell plate as described above; the mixture is incubated for 20 min, and an EC₇₀ dose of ghrelin is added to the cells. The assay proceeded as described above. Percent inhibition is determined by comparing the assay signal produced in the presence to that produced in the absence of test compound. Dose-response curves are plotted using GraphPad Prism software.

### C. In Vivo Food Intake and Body Weight Models.

1) Food intake and body weight in rats. Sprague Dawley rats are administered test compound one hour prior to onset of dark cycle (12 hours). Food intake is determined either by measurement of the remaining amount of preweighed food the morning following the dosing or by using a computerized system in which each rat's food is placed on a computer monitored balance. Cumulative food intake for 16 h post compound administration is measured. In some cases, food intake measurements are followed as long as 2 weeks. Body weight is measured daily; in some cases, adiposity is measured by DEXAscan analysis, tissue weights and plasma drug levels are measured. Animals can be dosed by a number of routes of administration. The routes of administration include intravenous (IV), intraperitoneal (IP), subcutaneous (SC) and intracerebral ventricular (ICV).

Compounds useful in the present invention decrease food intake acutely by at least 20% and/or decrease body weight in a 2 week period by at least 4% relative to placebo.

2) Food intake in diet induced obese mice. Male C57/B16J mice maintained on a high fat diet (30-60% fat calories) are dosed with test compound for 1 to 30 days. Food intake and body weight are measured overnight and sometimes daily as long as 30 days. Biochemical parameters relating to obesity, including leptin, insulin, triglyceride, free fatty acid, cholesterol and serum glucose levels and pharmacokinetic parameters may be determined. Animals can be dosed by a number of routes of administration. The routes of administration include intravenous (IV), intraperitoneal (IP), subcutaneous (SC) and intracerebral ventricular (ICV). Biochemical parameters relating to obesity, including leptin, insulin, triglyceride, free fatty acid, cholesterol and serum glucose levels are determined.

Compounds useful in the present invention decrease body weight by at least 4% relative to placebo.

Representative compounds of the present invention, including the compounds in Examples 1-345 were tested and found to bind to the ghrelin receptor, and were found to have IC₅₀ values less than 5 μM. Representative compounds of the present invention, including the compounds in Examples 1-345 were also tested in the functional assay and were found to antagonize the ghrelin receptor with EC₅₀ values less than 5 μM.

### Examples of Pharmaceutical Compositions

As a specific embodiment of an oral composition of a composition of the present invention, 5 mg of Example 1 is formulated with sufficient finely divided lactose to provide a total amount of 580 to 590 mg to fill a size O hard gelatin capsule.

As another specific embodiment of an oral composition of a compound of the present invention, 2.5 mg of Example 1 is formulated with sufficient finely divided lactose to provide a total amount of 580 to 590 mg to fill a size O hard gelatin capsule.

While the invention has been described and illustrated in reference to certain preferred embodiments thereof, those skilled in the art will appreciate that various changes, modifications and substitutions can be made therein without departing from the spirit and scope of the invention. For example, effective dosages other than the preferred doses as set forth hereinabove may be applicable as a consequence of variations in the responsiveness of the subject or mammal being treated for severity of bone disorders caused by resorption, or for other indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be limited only by the scope of the claims which follow and that such claims be interpreted as broadly as is reasonable.

1. A compound of structural formula I: 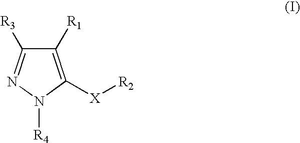
or a pharmaceutically acceptable salt thereof; wherein
X is selected from the group consisting of:
(1) bond,
(2) —(CH₂)ₘ—,
(3) —(CH₂)ₘC₂₋₆heterocycloalkyl-,
(4) —(CH₂)ₙC₂₋₆heterocycloalkyl-(CH₂)ₙ—NR⁶—,
(5) —NR⁶—(CH₂)ₙC₃₋₆cycloalkyl-(CH₂)ₙ—NR⁶—,
(6) —(CH₂)ₘNR⁶—,
(7) —NR⁶—(CH₂)ₘ—,
(8) —(CH₂)ₙ—NR⁶—(CH₂)ₘ—NR⁶—,
(9) —NR⁶—C₂₋₆alkenyl-,
(10) —NR⁶—C₂₋₆alkynyl-,
(11) —NR⁶-phenyl-,
(12) —NR⁶-phenyl-NR⁶—,
(13) —NR⁶—(CH₂)ₙ—C₂₋₆heterocycloalkyl-,
(14) —NR⁶—(CH₂)ₙ-heteroaryl-, and
(15) —NR⁶-heteroaryl-NR⁶—,
wherein alkenyl, alkynyl, cycloalkyl, heterocycloalkyl, phenyl, heteroaryl, and (CH₂) are unsubstituted or substituted with 1-4 substituents selected from oxo, halogen and C₁₋₄alkyl;
R¹ is selected from the group consisting of
(1) hydrogen,
(2) —CF₃,
(3) halogen,
(4) —C₁₋₈alkyl,
(5) —C₂₋₈alkenyl,
(6) —C₂₋₈alkynyl,
(7) —(CH₂)ₙOH,
(8) —(CH₂)ₙphenyl,
(9) —(CH₂)ₙheteroaryl,
(10) —(CH₂)ₙC₃₋₇cycloalkyl,
(11) —(CH₂)ₙC₂₋₉heterocycloalkyl,
(12) —(CH₂)ₙN(R⁶)CH₂-phenyl,
(13) —(CH₂)ₙN(R⁶)C(O)phenyl,
(14) —(CH₂)ₙN(R⁶)C(O)heteroaryl,
(15) —CN,
(16) —C(O)R⁵,
(17) —C(O)C₂₋₈alkenyl,
(18) —C(O)C₂₋₈alkynyl,
(19) —C(O)C₃₋₇cycloalkyl,
(20) —C(O)C₂₋₉heterocycloalkyl,
(21) —CO₂R⁵,
(22) —C(O)N(R⁶)₂, and
(23) —(CH₂)₃₋₇R²,
wherein alkyl, alkenyl, alkynyl, phenyl, heteroaryl, heterocycloalkyl, and cycloalkyl are unsubstituted or substituted with one to three groups independently selected from CF₃, C₁₋₄ alkoxy, C₁₋₄ alkyl, halogen and phenyl, wherein the phenyl substituent is unsubstituted or substituted with CF₃, C₁₋₄ alkoxy, C₁₋₄ alkyl and halogen;
R² is selected from the group consisting of
(1) hydrogen,
(2) —C₁₋₈alkyl,
(3) —C₂₋₈alkenyl,
(4) —C₂₋₈alkynyl,
(5) —(CH₂)ₙC₃₋₇cycloalkyl,
(6) —(CH₂)ₙC₂₋₉heterocycloalkyl,
(7) —(CH₂)ₙphenyl,
(8) —(CH₂)ₙnaphthyl,
(9) —(CH₂)ₙheteroaryl,
(10) —OR⁶,
(11) —C(O)R⁶,
(12) ═CH—N(R⁶)₂,
(13) —(CH₂)ₙN(R⁶)₂,
(14) —(CH₂)ₙN(R⁶)CO₂C₁₋₈alkyl,
(15) —(CH₂)ₙCO₂H,
(16) —C(O)C₁₋₈alkyl,
(17) —C(O)C₃₋₇cycloalkyl,
(18) —C(O)C₂₋₉heterocycloalkyl,
(19) —C(O)(CH₂)ₙaryl,
(20) —C(O)(CH₂)ₙheteroaryl,
(21) —C(O)CF₃,
(22) —C(O)(CH₂)ₙN(R⁶)₂,
(23) —C(O)N(R⁶)C₁₋₈alkyl,
(24) —C(O)N(R⁶)(CH₂)ₙC₃₋₇cycloalkyl,
(25) —C(O)N(R⁶)(CH₂)ₙC₂₋₇heterocycloalkyl,
(26) —C(O)N(R⁶)(CH₂)ₙphenyl,
(27) —C(O)N(R⁶)(CH₂)ₙnaphthyl,
(28) —C(O)N(R⁶)(CH₂)ₙheteroaryl,
(29) —C(S)N(R⁶)(CH₂)ₙphenyl,
(30) —CO₂C₁₋₈alkyl,
(31) —CO₂(CH₂)ₙC₃₋₇cycloalkyl,
(32) —CO₂(CH₂)ₙC₂₋₉heterocycloalkyl
(33) —CO₂(CH₂)ₙphenyl,
(34) —CO₂(CH₂)ₙnaphthyl,
(35) —CO₂(CH₂)ₙheteroaryl,
(36) —SO₂C₁₋₈alkyl,
(37) —SO₂C₃₋₇cycloalkyl,
(38) —SO₂C₂₋₉heterocycloalkyl,
(39) —SO₂phenyl,
(40) —SO₂naphthyl,
(41) —SO₂heteroaryl,
(42) —S(O)N(R⁶)phenyl,
(43) —S—C₁₋₈alkyl,
(44) —S—C₃₋₇cycloalkyl,
(45) —S—C₂₋₉heterocycloalkyl,
(46) —S-phenyl,
(47) —S-naphthyl, and
(48) —S-heteroaryl,
wherein alkyl, alkenyl, alkynyl, cycloalkyl, heterocycloalkyl, aryl, phenyl, naphthyl, heteroaryl, and (CH₂) are unsubstituted or substituted with one to four substituents independently selected from R⁷, and wherein two C₁₋₄ alkyl substituents on the same (CH₂) carbon may cyclize to form a 3- to 6-membered ring, provided that when X is a bond or —(CH₂)ₘ then R² is not hydrogen, —C₁₋₈alkyl, —C₂₋₈alkenyl, —C₂₋₈alkynyl, —(CH₂)ₙC₃₋₇cycloalkyl, —C₂₋₉heterocycloalkyl, -phenyl, -benzyl, -naphthyl, -heteroaryl, —OR⁶, —C(O)R⁶, or —S—C₁₋₈alkyl, further provided that when X is a bond R² is not —NH₂, —CO₂C₁₋₈alkyl, —CO₂C₃₋₇cycloalkyl, —CO₂(CH₂)₀₋₁phenyl, and provided that when X is —(CH₂)ₘNR⁶— then R² is not —C(O)R⁶;
R³ is selected from the group consisting of:
(1) —C₁₋₈alkyl,
(2) —(CH₂)ₙ-phenyl,
(3) —(CH₂)ₙ-naphthyl,
(4) —(CH₂)ₙC₃₋₇cycloalkyl,
(5) —C(O)C₁₋₈alkyl,
(6) —CO₂R⁵,
(7) —C(O)N(R⁶)OC1-8alkyl,
(8) —C(O)C₁₋₄alkenylphenyl,
(9) —C(O)C₁₋₄alkynylphenyl,
(10) —C(O)phenyl,
(11) —C(O)naphthyl,
(12) —C(O)heteroaryl, and
(13) —C(O)C₃₋₇cycloalkyl,
wherein alkyl, alkenyl, alkynyl, phenyl, naphthyl, heteroaryl, and cycloalkyl are unsubstituted or substituted with one to three groups independently selected from R⁸, and each (CH₂)ₙ is unsubstituted or substituted with 1 to 2 groups independently selected from: C₁₋₄alkyl, —OH, halogen, and C₁₋₄alkenyl;
R⁴ is selected from the group consisting of:
(1) —(CH₂)ₙ-phenyl,
(2) —(CH₂)ₙ-naphthyl,
(3) —(CH₂)ₙ-heteroaryl,
(4) —(CH₂)ₙC₂₋₉heterocycloalkyl,
(5) —(CH₂)ₙC₃₋₇cycloalkyl, and
(6) —S(O)₂phenyl,
wherein phenyl, naphthyl, heteroaryl, heterocycloalkyl, cycloalkyl and (CH₂) are unsubstituted or substituted with one to three groups independently selected from R⁹;
each R⁵ is independently selected from the group consisting of
(1) —C₁₋₈alkyl,
(2) —(CH₂)ₙphenyl, and
(3) —(CH₂)ₙheteroaryl,
wherein each carbon in —C₁₋₈alkyl is unsubstituted or substituted with one to three groups independently selected from C₁₋₄alkyl;
each R⁶ is independently selected from the group consisting of
(1) hydrogen,
(2) —C₁₋₈ alkyl,
(3) —C₂₋₈alkenyl,
(4) —C₂₋₈alkynyl,
(5) (CH₂)ₙphenyl,
(6) —C₂₋₈alkenylphenyl, and
(7) —(CH₂)ₙCO₂H,
wherein alkyl, alkenyl, alkynyl, and (CH₂)ₙ are unsubstituted or each carbon is substituted with 1 or 2 substituents independently selected from —OC₁₋₄alkyl, and —C₁₋₄alkyl; and phenyl is unsubstituted or substituted with 1-3 groups selected from —OC₁₋₄alkyl, and —C₁₋₄alkyl;
each R⁷ is independently selected from the group consisting of:
(1) halogen,
(2) oxo,
(3) ═NH,
(4) —CN,
(5) —CF₃,
(6) —OCF₃,
(7) —C₁₋₆alkyl,
(8) —C₂₋₆ alkenyl,
(9) —C₂₋₆ alkynyl,
(10) —(CH₂)ₙC₃₋₆cycloalkyl,
(11) —(CH₂)ₙC₂₋₉heterocycloalkyl,
(12) —(CH₂)ₙOR⁶,
(13) —(CH₂)ₙCO₂R⁶,
(14) —(CH₂)ₙCO₂(CH₂)ₙphenyl;
(15) —(CH₂)ₙphenyl;
(16) —(CH₂)ₙ—O-phenyl;
(17) —(CH₂)ₙnaphthyl,
(18) —(CH₂)ₙ-heteroaryl,
(19) —N(R⁶)₂,
(20) —NR⁶C(O)R⁶,
(21) —NR⁶C(O)₂R⁶,
(22) —C(O)phenyl,
(23) —C(O)heteroaryl,
(24) —SR⁵,
(25) —SO₂C₁₋₆alkyl, and
(26) —SO₂N(R⁶)₂,
wherein alkyl, alkenyl, alkynyl, phenyl, heteroaryl, heterocycloalkyl, naphthyl, cycloalkyl, and (CH₂)ₙ are unsubstituted or substituted with one to three groups independently selected from oxo, halogen, C₁₋₄ alkyl and OR⁵;
each R⁸ is independently selected from the group consisting of:
(1) —C₁₋₆alkyl,
(2) —C₁₋₆alkenyl,
(3) —C₁₋₆alkynyl,
(4) —C₁₋₆alkoxy,
(5) —C₃₋₆cycloalkyl,
(6) —(CH₂)ₙ-phenyl, unsubstituted or substituted with halogen,
(7) —O—(CH₂)ₙ-phenyl,
(8) —CN,
(9) —OH,
(10) halogen,
(11) —CF₃,
(12) —NH₂,
(13) —N(C₁₋₆alkyl)₂,
(14) —NO₂, and
(15) —SC₁₋₆alkyl;
each R⁹ is independently selected from the group consisting of:
(1) halogen,
(2) —C₁₋₆alkyl,
(3) —C₂₋₆alkenyl,
(4) —C₂₋₆alkynyl,
(5) phenyl,
(6) —CH₂phenyl,
(7) —(CH₂)ₙOR⁶,
(8) —CN,
(9) —OCF₃,
(10) —CF₃,
(11) —NO₂,
(12) —NR⁵COR⁵,
(13) —CO₂R⁵, and
(14) —CO₂H;
n is 0, 1, 2, 3, 4, 5, 6, 7 or 8; and
m is 1, 2, 3, 4, 5, 6, 7 or 8.

2. The compound of claim 1 wherein R¹ is selected from the group consisting of: halogen, —C₁₋₄alkyl, and —CN; or a pharmaceutically acceptable salt thereof.

3. The compound of claim 4 wherein R¹ is —CN; or a pharmaceutically acceptable salt thereof.

4. The compound of claim 1 wherein R³ is selected from the group consisting of:
(1) —C(O)N(CH₃)OCH₃,
(2) —C(O)phenyl, and
(3) —C(O)-(1,3-benzodioxole),
wherein phenyl is substituted with 1-3 substituents selected from: CF₃, Br and CH₃; or a pharmaceutically acceptable salt thereof.

5. The compound of claim 1 wherein R³ is —C(O)phenyl, wherein phenyl is substituted with CF₃ or CH₃; or a pharmaceutically acceptable salt thereof.

6. The compound of claim 1 wherein R⁴ is selected from the group consisting of: phenyl, naphthyl, and heteroaryl, wherein phenyl, naphthyl, heteroaryl, and (CH₂) are unsubstituted or substituted with one to three groups independently selected from halogen, —C₁₋₆alkyl, —C₂₋₆alkenyl, —C₂₋₆alkynyl, phenyl, —CH₂-phenyl, —(CH₂)ₙOR⁶, —CN, —OCF₃, —CF₃, —NO₂, —NR⁵COR⁵, —CO₂R⁵, and —CO₂H; or a pharmaceutically acceptable salt thereof.

7. The compound of claim 1 wherein R⁴ is 4-chlorophenyl or 4-fluorophenyl; or a pharmaceutically acceptable salt thereof.

8. The compound of claim 1 wherein X is selected from the group consisting of
(1) —(CH₂)ₙ—C₂₋₉heterocycloalkyl-,
(2) —(CH₂)ₙ—C₂₋₉heterocycloalkyl-(CH₂)ₙ—NR⁶—,
(3) —NR⁶—(CH₂)ₙ—C₃₋₆cycloalkyl-(CH₂)ₙ—NR⁶—,
(4) —(CH₂)ₙ—NR⁶—,
(5) —NR⁶—(CH₂)ₘ—,
(6) —NR⁶—(CH₂)ₘ—NR⁶—,
(7) —NR⁶—C₂₋₆alkenyl-,
(8) —NR⁶-phenyl-NR⁶—,
(9) —(CH₂)ₙNR⁶—C₂₋₉heterocycloalkyl-, and
(10) —NR⁶—(CH₂)ₙ-heteroaryl-,
wherein alkenyl, heterocycloalkyl, phenyl, heteroaryl, and (CH₂)ₙ are unsubstituted or substituted with 1-4 substituents selected from oxo, halogen and C₁₋₄alkyl; or a pharmaceutically acceptable salt thereof.

9. The compound of claim 1 wherein X is selected from the group consisting of:
(1) —(CH₂)ₙC₂₋₆heterocycloalkyl-NR⁶—,
(2) —NR⁶—C₃₋₆cycloalkyl-NR⁶—,
(3) —NR⁶—(CH₂)ₘ—NR⁶—, and
(4) —NR⁶—(CH₂)ₘ—,
wherein cycloalkyl, heterocycloalkyl, heteroaryl, and (CH₂)ₙ are unsubstituted or substituted with 1-4 substituents selected from oxo, halogen and C₁₋₄alkyl; or a pharmaceutically acceptable salt thereof.

10. The compound of claim 1 wherein R² is selected from the group consisting of:
(1) hydrogen,
(2) —C₁₋₈alkyl,
(3) —(CH₂)ₙC₂₋₉heterocycloalkyl,
(4) —(CH₂)ₙphenyl,
(5) —(CH₂)ₙnapthyl,
(6) —(CH₂)ₙheteroaryl,
(7) —OR⁶,
(8) —(CH₂)ₙN(R⁶)₂,
(9) —(CH₂)ₙN(R⁶)CO₂C₁₋₈alkyl,
(10) —C(O)C₁₋₈alkyl,
(11) —C(O)C₃₋₇cycloalkyl,
(12) —C(O)C₂₋₉heterocycloalkyl,
(13) —C(O)(CH₂)ₙaryl,
(14) —C(O)(CH₂)ₙheteroaryl,
(15) —C(O)CF₃,
(16) —C(O)N(R⁶)C₁₋₈alkyl,
(17) —C(O)N(R⁶)(CH₂)ₙphenyl,
(18) —C(O)N(R⁶)(CH₂)ₙnaphthyl,
(19) —CO₂C₁₋₈alkyl,
(20) —CO₂(CH₂)ₙphenyl,
(21) —SO₂C₁₋₈alkyl,
(22) —SO₂phenyl,
(23) —S(O)N(R⁶)phenyl, and
(24) —S-phenyl,
wherein alkyl, cycloalkyl, heterocycloalkyl, aryl, phenyl, naphthyl, heteroaryl, and (CH₂) are unsubstituted or substituted with one to four substituents independently selected from R⁷, and wherein two C₁₋₄ alkyl substituents on the same (CH₂) carbon may cyclize to form a 3- to 6-membered ring, provided that when X is a bond or —(CH₂)ₘ then R² is not hydrogen, —C₁₋₈alkyl, —C₂₋₈alkenyl, —C₂₋₈alkynyl, —(CH₂)ₙC₃₋₇cycloalkyl, —C₂₋₉heterocycloalkyl, -phenyl, -benzyl, -naphthyl, -heteroaryl, —OR⁶, —C(O)R⁶, or —S—C₁₋₈alkyl, further provided that when X is a bond R² is not —NH₂, —CO₂C₁₋₈alkyl, —CO₂C₃₋₇cycloalkyl, —CO₂(CH₂)₀₋₁phenyl, and provided that when X is —(CH₂)ₘNR⁶— then R² is not hydrogen or —C(O)R⁶; or a pharmaceutically acceptable salt thereof.

11. The compound of claim 1 wherein R² is selected from the group consisting of:
(1) —(CH₂)ₙphenyl,
(2) —(CH₂)ₙheteroaryl,
(3) —C(O)phenyl, and
(4) —C(O)heteroaryl,
wherein phenyl and heteroaryl are unsubstituted or substituted with one to three substituents independently selected from R⁷, and wherein each (CH₂) carbon is unsubstituted or substituted with one or two substituents independently selected from halogen, C₁₋₄alkyl, oxo, —(CH₂)ₙOR⁵, —(CH₂)ₙCO₂R⁵, or two C₁₋₄ alkyl substituents on the same (CH₂) carbon can cyclize to form a 3- to 6-membered ring, provided that when X is a bond or —(CH₂)ₘ then R² is not -phenyl, -benzyl, or -heteroaryl, and provided that when X is —(CH₂)ₘNR⁶— then R² is not —C(O)phenyl or C(O)heteroaryl; or a pharmaceutically acceptable salt thereof.

12. The compound of claim 1 wherein
X is selected from the group consisting of:
(1) —(CH₂)-pyrrolidinyl-NH—,
(2) —NH-cyclobutyl-NH—,
(3) —NH—(CH₂)₃—NH—, and
(4) —NH—(CH₂)₃—;
R¹ is —CN;
R² is selected from the group consisting of:
(1) —(CH₂)ₙphenyl,
(2) —(CH₂)ₙheteroaryl,
(3) —C(O)phenyl, and
(4) —C(O)heteroaryl,
wherein phenyl and heteroaryl are unsubstituted or substituted with one to three substituents independently selected from R⁷, and wherein each (CH₂) carbon is unsubstituted or substituted with one or two substituents independently selected from halogen, C₁₋₄alkyl, oxo, —(CH₂)ₙOR⁵, —(CH₂)ₙCO₂R⁵, or two C₁₋₄alkyl substituents on the same (CH₂) carbon can cyclize to form a 3- to 6-membered ring, provided that when X is a bond or —(CH₂)ₘ then R² is not -phenyl, -benzyl, or -heteroaryl, and provided that when X is —(CH₂)ₘNR⁶— then R² is not —C(O)phenyl or C(O)heteroaryl;
R³ is —C(O)phenyl, wherein phenyl is substituted with CF₃ or CH₃; and
each R⁷ is independently selected from the group consisting of: Br, I, F, Cl, oxo, ═NH, —CN, —CF₃, —CH₃, —CH₂CH₃, —CH(CH₃)₂, —C(CH₃)₃, cyclopropyl, succinamide, —CH₂OCH₃, —CH₂OH, —OCH₃, —OCH₂CH₃, —O(CH₂)₃CH₃, —OCH(CH₃)₂, —CO₂CH₃, —CO₂H, -phenyl, —CH₂-phenyl, —O-phenyl, pyridine, pyrazole, tetrazole, —N(CH₃)₂, —NH₂, —NHC(O)CH₃, —SCH₃, —SO₂CH₃, and —SO₂NH₂, wherein the R⁷ substituents are unsubstituted or substituted with one to three groups independently selected from oxo, halogen, C₁₋₄ alkyl and OR⁵;
or a pharmaceutically acceptable salt thereof.

13. The compound of claim 12 selected from the group consisting of: 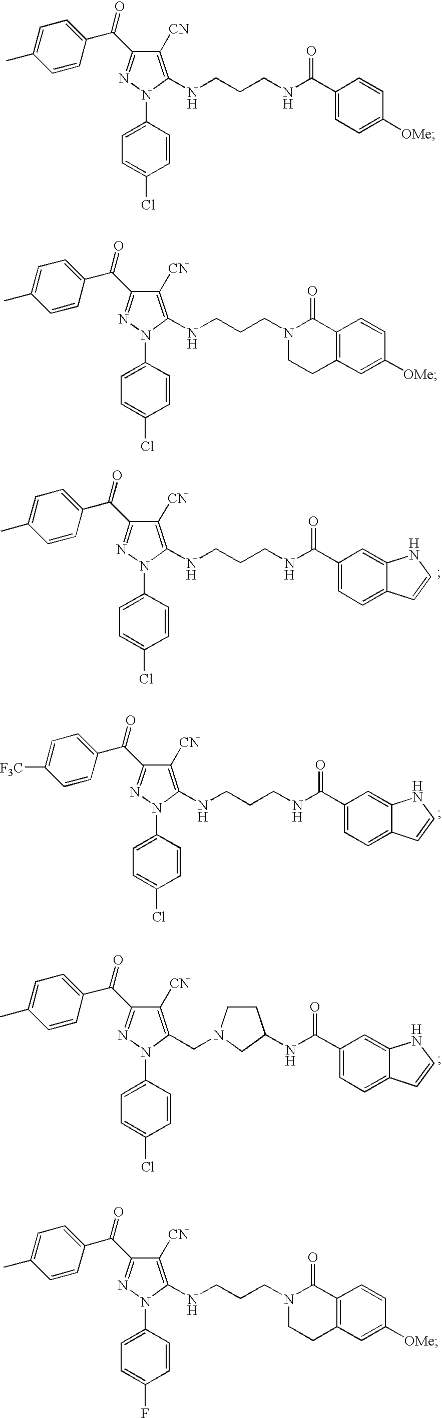 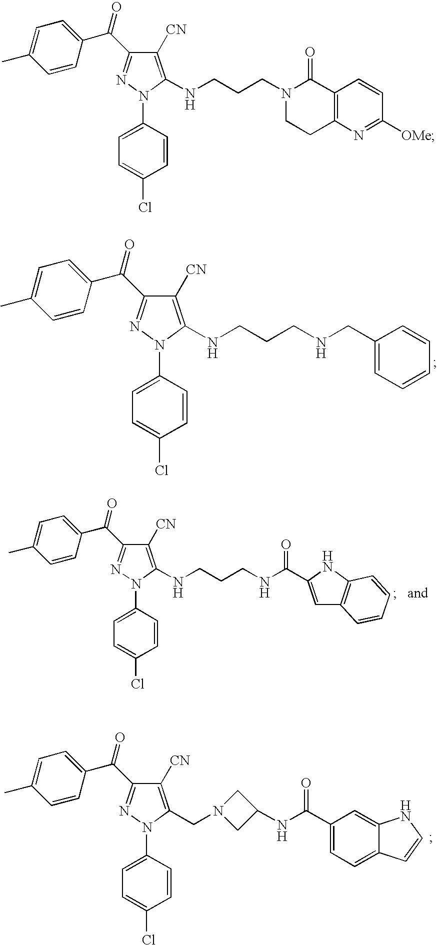
or a pharmaceutically acceptable salt thereof.

14. A pharmaceutical composition which comprises a compound of claim 1, or a pharmaceutically acceptable salt thereof, and a pharmaceutically acceptable carrier.

15. A method for the treatment or prevention of disorders, diseases or conditions responsive to the modulation of the ghrelin receptor in a subject in need thereof which comprises administering to the subject a therapeutically or prophylactically effective amount of a compound according to claim 1, or a pharmaceutically acceptable salt thereof.

16. A method for the treatment or prevention of obesity in a subject in need thereof which comprises administering to the subject a therapeutically or prophylactically effective amount of a compound according to claim 1, or a pharmaceutically acceptable salt thereof.

17. A method for the treatment or prevention of diabetes mellitus in a subject in need thereof comprising administering to the subject a therapeutically or prophylactically effective amount of a compound according to claim 1, or a pharmaceutically acceptable salt thereof.

18. A method for the treatment or prevention of metabolic syndrome in a subject in need thereof comprising administering to the subject a therapeutically or prophylactically effective amount of a compound according to claim 1, or a pharmaceutically acceptable salt thereof.

19. (canceled)

20. (canceled)
